(19) United States
(12) Patent Application Publication
(10) Pub. No.: US 2025/0027126 A1
(43) Pub. Date: Jan. 23, 2025

(54) IMPROVED METHODS AND ENZYMES

(71) Applicant: GIVAUDAN SA, VERNIER (CH)

(72) Inventors: Eric EICHHORN, Kemptthal (CH); Alessandro SCARDUA, Biassono (IT); Christophe ULLMANN, Nimes (FR)

(21) Appl. No.: 18/703,116

(22) PCT Filed: Oct. 20, 2022
(86) PCT No.: PCT/EP2022/079172

§ 371 Date: Apr. 19, 2024

(30) Foreign Application Priority Data
Oct. 21, 2021 (GB) 2115120.4
Mar. 30, 2022 (GB) 2204546.2

(51) Int. Cl. C12P 17/08; A61K 8/49; A61Q 13/00; C07D 313/20; C07D 493/08; C12N 9/90

(52) U.S. Cl. CPC C12P 17/08; A61K 8/498; A61Q 13/00; C07D 313/20; C07D 493/08; C12N 9/90; C12Y 504/99017

(57) ABSTRACT

Improved methods of making amberketal and amberketal homologues and compositions comprising same, improved squalene-hopene cyclase (SHC) enzymes to be used in said methods, nucleic acid constructs and vectors encoding said enzymes, and host cells expressing said enzymes.

### FIELD

The present disclosure generally relates to improved methods of making amberketal and amberketal homologues. The disclosure further relates to improved SHC enzymes to be used in said methods, nucleic acid constructs and vectors encoding said enzymes, and host cells expressing said enzymes.

### BACKGROUND

Amberketal provides a powerful and tenacious ambery and woody odour that is useful in fragrance compositions, alone or in combination with other woody or ambery ingredients. Amberketal is traditionally prepared from manool via a number of chemical transformations. However, the supply of natural manool is limited. WO2021/209482 discloses a method for producing amberketal and amberketal homologues from polyunsaturated alcohols using a squalene-hopene cyclase (SHC) enzyme.

### SUMMARY

An aspect of the disclosure relates to a method for making a compound of formula (I)

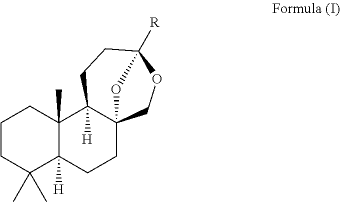

wherein the method comprises contacting a compound of formula (II)

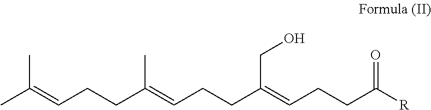

with a squalene-hopene cyclase (SHC) enzyme comprising an amino acid sequence having at least 70% identity or similarity with the sequence of SEQ ID NO: 1, wherein the SHC enzyme comprises one or more amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1, and wherein R is selected from H and a C₁-C₄ alkyl.

In some embodiments of a method for making a compound of formula (I), the method is such that the compound of formula (II) is such that the double bond between C-8 and C-9 is in E-configuration and the double bond between C-4 and C-5 is in Z-configuration (E,Z-isomer).

A further aspect of the disclosure relates to a method for making a mixture comprising a compound of formula (I)

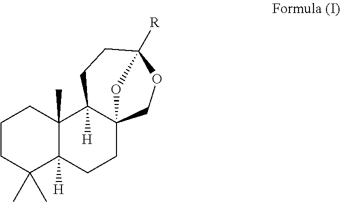

wherein the method comprises contacting a mixture comprising a compound of formula (II) and a compound of formula (IIa)

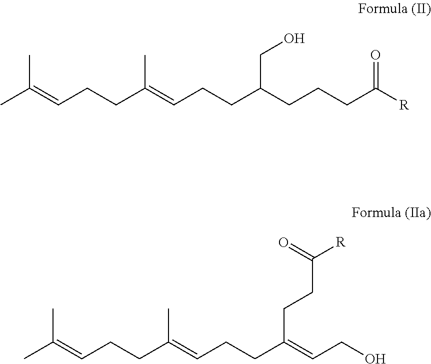

with a squalene-hopene cyclase (SHC) enzyme comprising an amino acid sequence having at least 70% identity or similarity with the sequence of SEQ ID NO: 1 or SEQ ID NOs: 43-49, preferably having at least 70% identity or similarity with the sequence of SEQ ID NO: 1 and comprising one or more amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1, and wherein R is selected from H and a C₁-C₄ alkyl.

In some embodiments of a method for making a mixture comprising a compound of formula (I), the method is such that the mixture comprising a compound of formula (I) further comprises a compound of formula (Ia)

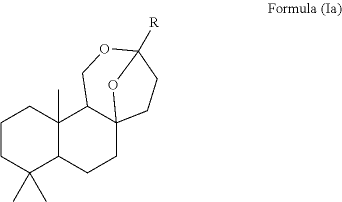

wherein R is selected from H and a C₁-C₄ alkyl. In some embodiments, the compound of formula (Ia) has the configuration of formula (V)

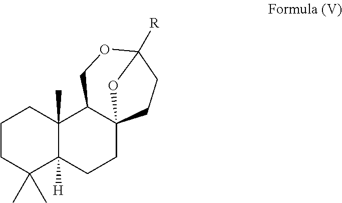

wherein R is selected from H and a C₁-C₄ alkyl.

In some embodiments of a method for making a mixture comprising a compound of formula (I), the method is such that the mixture comprising a compound of formula (II) and a compound of formula (IIa) comprises any one of the following:
i) a compound of formula (II) that is such that the double bond between C-8 and C-9 is in E-configuration and the double bond between C-4 and C-5 is in Z-configuration (E,Z-isomer)
ii) a compound of formula (II) that is such that the double bond between C-8 and C-9 is in E-configuration and the double bond between C-4 and C-5 is in E-configuration (E,E-isomer)
iii) a compound of formula (IIa) that is such that the double bond between C-6 and C-7 is in E-configuration and the double bond between C-2 and C-3 is in Z-configuration (E,Z-isomer)
iv) a compound of formula (IIa) that is such that the double bond between C-6 and C-7 is in E-configuration and the double bond between C-2 and C-3 is in E-configuration (E,E-isomer)
v) a compound of formula (II) that is such that the double bond between C-8 and C-9 is in E-configuration and the double bond between C-4 and C-5 is in Z-configuration (E,Z-isomer) and a compound of formula (II) that is such that the double bond between C-8 and C-9 is in E-configuration and the double bond between C-4 and C-5 is in E-configuration (E,E-isomer)
vi) a compound of formula (IIa) that is such that the double bond between C-6 and C-7 is in E-configuration and the double bond between C-2 and C-3 is in Z-configuration (E,Z-isomer) and a compound of formula (IIa) that is such that the double bond between C-6 and C-7 is in E-configuration and the double bond between C-2 and C-3 is in E-configuration (E,E-isomer)
vii) any combination of i)-vi).

In some embodiments of a method for making a mixture comprising a compound of formula (I), the method is such that the mixture comprising a compound of formula (II) and a compound of formula (IIa) comprises:
a compound of formula (II) that is such that the double bond between C-8 and C-9 is in E-configuration and the double bond between C-4 and C-5 is in Z-configuration (E,Z-isomer)
a compound of formula (II) that is such that the double bond between C-8 and C-9 is in E-configuration and the double bond between C-4 and C-5 is in E-configuration (E, E-isomer)
a compound of formula (IIa) that is such that the double bond between C-6 and C-7 is in E-configuration and the double bond between C-2 and C-3 is in Z-configuration (E,Z-isomer), and;
a compound of formula (IIa) that is such that the double bond between C-6 and C-7 is in E-configuration and the double bond between C-2 and C-3 is in E-configuration (E,E-isomer).

In some embodiments of a method for making a compound of formula (I) and a method for making a mixture comprising a compound of formula (I), the compound of formula (III)

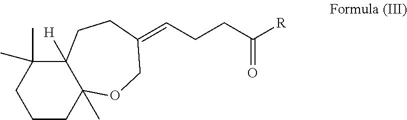

is made as a by-product,
wherein R is selected from H and a C₁-C₄ alkyl.

In some embodiments of a method for making a compound of formula (I) and a method for making a mixture comprising a compound of formula (I), a compound having the relative configuration shown in formula (IIIa) is made as a by-product:

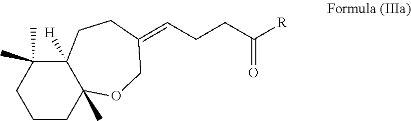

wherein R is selected from H and a C₁-C₄ alkyl.

In some embodiments of a method for making a mixture comprising a compound of formula (I), a compound of formula (VI)

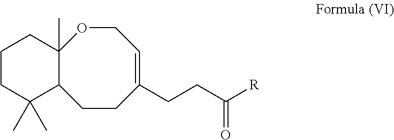

is made as a by-product,
wherein R is selected from H and a C₁-C₄ alkyl.

In some embodiments of a method for making a mixture comprising a compound of formula (I), a compound having the relative configuration shown in formula (VIa) is made as a by-product:

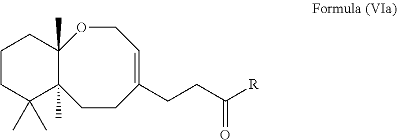

wherein R is selected from H and a C₁-C₄ alkyl.

In some embodiments of a method for making a compound of formula (I) and a method for making a mixture comprising a compound of formula (I), R is methyl.

In some embodiments of a method for making a compound of formula (I) and a method for making a mixture comprising a compound of formula (I), the SHC enzyme comprises an amino acid sequence having at least 70% identity or similarity with the sequence of SEQ ID NO: 1, and the SHC enzyme comprises one to seven, preferably two to six, more preferably three to five amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1.

In some embodiments of a method for making a compound of formula (I) and a method for making a mixture comprising a compound of formula (I), the SHC enzyme comprises one or more amino acid substitutions relative to SEQ ID NO: 1 at one or more positions corresponding to position 2, 5, 35, 166, 211, 212, 355, 483, and 539 in SEQ ID NO: 1.

In some embodiments of a method for making a compound of formula (I) and a method for making a mixture comprising a compound of formula (I), the SHC enzyme comprises one or more amino acid substitutions relative to SEQ ID NO: 1 at one or more positions corresponding to position 2, 5, 35, 166, 211, 212, 483, and 539, preferably corresponding to position 2, 5, 35, 166, 211, 483, and 539 in SEQ ID NO: 1.

In some embodiments of a method for making a compound of formula (I) and a method for making a mixture comprising a compound of formula (I), the SHC enzyme comprises an amino acid substitution relative to SEQ ID NO: 1 selected from the following:
(i) an asparagine (N) residue at a position corresponding to position 2 in SEQ ID NO: 1;
(ii) a proline (P) residue at a position corresponding to position 5 in SEQ ID NO: 1;
(iii) an alanine (A) residue at a position corresponding to position 35 in SEQ ID NO: 1;
(iv) an threonine (T) residue at a position corresponding to position 116 in SEQ ID NO: 1;
(v) an alanine (A) residue at a position corresponding to position 166 in SEQ ID NO: 1;
(vi) a valine (V) residue at a position corresponding to position 211 in SEQ ID NO: 1;
(vii) an arginine (R) residue at a position corresponding to position 212 in SEQ ID NO: 1;
(viii) a methionine (M) residue at a position corresponding to position 317 in SEQ ID NO: 1;
(ix) a threonine (T) residue at a position corresponding to position 355 in SEQ ID NO: 1;
(x) a threonine (T) residue at a position corresponding to position 382 in SEQ ID NO: 1;
(xi) a valine (V) residue at a position corresponding to position 399 in SEQ ID NO: 1;
(xii) a cysteine (C) residue at a position corresponding to position 483 in SEQ ID NO: 1;
(xiii) a histidine (H) residue at a position corresponding to position 539 in SEQ ID NO: 1;
(xiv) an alanine (A) residue at a position corresponding to position 585 in SEQ ID NO: 1; or
(xv) any combination thereof.

In some embodiments of a method for making a compound of formula (I) and a method for making a mixture comprising a compound of formula (I), the SHC enzyme comprises an amino acid substitution relative to SEQ ID NO: 1 selected from the following corresponding positions in SEQ ID NO: 1:
(i) I2N, T35A, A355T, and L539H;
(ii) T166A;
(iii) I2N and Y483C;
(iv) I2N, Y483C, and L539H;
(v) I2N, L5P, T35A, L539H;
(vi) I2N, L5P, T35A, and Y483C;
(vii) I2N, L5P, T35A, T166A, and L539H;
(viii) I2N, L5P, T35A, T166A, E211V, and L539H
(ix) I2N, L5P, T35A, E211V, S212R, Y483C, and L539H
(x) I2N, T166A, and Y483C;
(xi) I2N, T166A, Y483C, and L539H;
(xii) I2N, T166A, E211V, and Y483C; or
(xiii) I2N, T166A, E211V, Y483C, and L539H.

In some embodiments of a method for making a compound of formula (I) and a method for making a mixture comprising a compound of formula (I), the SHC enzyme comprises the following amino acid substitutions relative to SEQ ID NO: 1: I2N and T166A.

In some embodiments of a method for making a compound of formula (I) and a method for making a mixture comprising a compound of formula (I), the SHC enzyme further comprises one or more substitutions relative to SEQ ID NO: 1 selected from L5P, T35A, E211V, Y483C, and L539H.

In some embodiments of a method for making a compound of formula (I) and a method for making a mixture comprising a compound of formula (I), the SHC enzyme further comprises an amino acid sequence that is at least 90% identical to any one of SEQ ID NOs: 4, 6, 8, 10, 12, 14, 16, 18, 20, 22, 24, 26, 28, 30, 32, 34, 36, 38, 40 or 42, preferably SEQ ID NOs: 4, 6, 18, 20, 22, 24, 30, 32, 34, 36, 38, 40 or 42, more preferably SEQ ID NOs: 30, 32, 34, 36, 38, 40 or 42, most preferably SEQ ID NOs: 30, 38, 40, 42.

A further aspect of the disclosure relates to a nucleic acid molecule comprising a nucleotide sequence encoding a squalene hopene cyclase (SHC) enzyme as described in any of the methods for making a compound of formula (I) and methods for making a mixture comprising a compound of formula (I).

A further aspect of the disclosure relates to a vector comprising a nucleic acid molecule according to the disclosure.

A further aspect of the disclosure relates to a host cell comprising a nucleic acid molecule according to the disclosure or a vector according to the disclosure.

A further aspect of the disclosure relates to a squalene hopene cyclase (SHC) enzyme as described in any of the methods for making a compound of formula (I) and methods for making a mixture comprising a compound of formula (I).

A further aspect of the disclosure relates to a composition comprising a compound of formula (I) and a compound of formula (Ia), wherein said composition is obtained by or is obtainable by for making a mixture comprising a compound of formula (I) according to the disclosure.

In some embodiments, the composition is such that the compound of formula (I) and the compound of formula (Ia) are in a solid form, preferably in an amorphous or crystalline form. In some embodiments, the composition is such that the compound of formula (Ia) has the configuration of formula (V).

A further aspect of the disclosure relates to use of a composition according to the disclosure for the manufacture of a fragrance composition or a consumer product.

A further aspect of the disclosure relates to a fragrance composition or a consumer product comprising the composition according to the disclosure.

A further aspect of the disclosure relates to a mixture comprising the product obtainable by the process asv described in any of the methods for making the compounds of the disclosure wherein the mixture comprises I, Ia, III, IIIa, IV, IVa, V, Va, VI and/or VIa.

A further aspect of the disclosure relates to a composition according to the disclosure wherein the composition comprises a compound of formula (I) and/or a compound of formula (Ia) and further comprises III, IIIa, IV, IVa, V, Va and VI and/or VIa.

### DESCRIPTION

There is still a need to provide new, more efficient, cost-effective, and sustainable methods for producing amberketal and amberketal homologues. The financial viability and sustainability of amberketal and amberketal homologue production methods can be enhanced by obtaining improved substrate conversion rates and product yields, decreased byproduct yields, and improved overall reaction performance under industrially relevant conditions. Accordingly, there is still a need for improved amberketal and amberketal homologue production processes. Accordingly, there is still a need for improved SHC enzymes and host cells expressing said enzymes for producing amberketal and amberketal homologues.

The present inventors have surprisingly found that the squalene-hopene cyclase (SHC) enzymes described herein are able to convert a compound of formula (IIa) to a compound of formula (Ia) as described later herein. They are further able to convert a compound of formula (II) and/or a compound of formula (IIa), wherein the compound of formula (II) and the compound of formula (IIa) are comprised in a mixture to, respectively, a compound of formula (I) and a compound of formula (Ia). Further, substitution of amino acid residues corresponding to one or more specific positions of a squalene-hopene cyclase (SHC) enzyme results in improved conversion of a compound of formula (II) to a compound of formula (I) and/or improved conversion of a compound of formula (IIa) to a compound of formula (Ia), as described later herein.

Particularly, as elaborated elsewhere herein and in the experimental part, the methods, enzymes, and host cells described herein exert at least one, at least two, or all of the following advantageous effects:
Improved conversion rate of a compound of formula (II) and/or of a compound of formula (IIa)
Improved yield of a compound of formula (I) and/or a compound of formula (Ia)
Improved reaction performance (e.g., conversion rate, productivity, yield at high substrate concentration

Accordingly, the aspects and embodiments of the present disclosure solve at least some of the problems and needs as discussed herein.

### Methods

Methods described herein may involve the enzymatic conversion of a compound of formula (II) to a compound of formula (I) by an SHC enzyme of the disclosure. Methods described herein may involve the enzymatic conversion of a compound of formula (IIa) to a compound of formula (Ia) by an SHC enzyme of the disclosure. Methods described herein may involve the enzymatic conversion of a compound of formula (II) and/or a compound of formula (IIa), wherein the compound of formula (II) and the compound of formula (IIa) are comprised in a mixture, to, respectively, a compound of formula (I) and/or a compound of formula (Ia), or to a mixture comprising a compound of formula (I) and/or a compound of formula (Ia).

Accordingly, in an aspect, the disclosure provides a method for making a compound of formula (I)

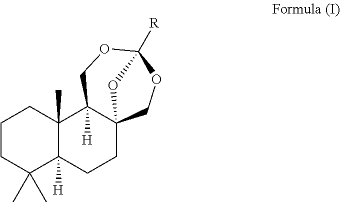

wherein the method comprises contacting a compound of formula (II)

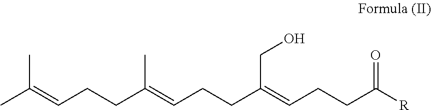

with a squalene-hopene cyclase (SHC) enzyme as described herein.

In an aspect, the disclosure provides a method for making a compound of formula (Ia)

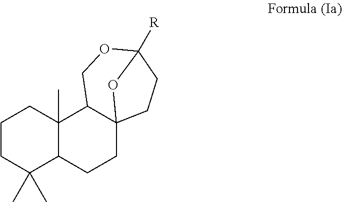

wherein the method comprises contacting a compound of formula (IIa)

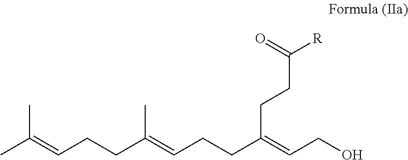

with a squalene-hopene cyclase (SHC) enzyme as described herein.

In an aspect, the disclosure provides a method for making a mixture comprising a compound of formula (I) and/or a compound of formula (Ia), wherein the method comprises contacting a compound of formula (II) and/or a compound of formula (IIa) with a squalene-hope cyclase (SHC) enzyme as described herein. A compound of formula (II) and/or a compound of formula (IIa) may be present in a mixture.

In some embodiments, the squalene-hope cyclase (SHC) enzyme comprises an amino acid sequence having at least 30%, 40%, 50%, 60%, or 70%, preferably at least 70%, identity or similarity with the sequence of SEQ ID NO: 1 or SEQ ID NOs: 43-49.

In preferred embodiments, the squalene-hopene cyclase (SHC) enzyme comprises an amino acid sequence having at least 30%, 40%, 50%, 60%, or 70%, preferably at least 70%, identity or similarity with the sequence of SEQ ID NO: 1, preferably wherein the SHC enzyme comprises one or more amino acid substitutions relative to SEQ ID NO: 1. Preferably, the one or more amino acid substitutions relative to SEQ ID NO: 1 are at one or more positions corresponding to position 2, 5, 35, 116, 166, 211, 212, 317, 355, 382, 399, 483, 539, and 585 in SEQ ID NO: 1.

SHC enzymes according to the disclosure are described in more detail later herein.

R in all formulas described herein may be selected from H (hydrogen) and a C₁-C₄ alkyl. In some embodiments, R is H (hydrogen). In some embodiments, R is ethyl. In some embodiments, R is n-propyl. In some embodiments, R is iso-propyl. In preferred embodiments, R is methyl.

Accordingly, in some embodiments, there is provided a method for making a compound of formula (I), wherein the method comprises contacting a compound of formula (II) with a squalene-hopene cyclase (SHC) enzyme comprising an amino acid sequence having at least 70% identity or similarity with the sequence of SEQ ID NO: 1, wherein the SHC enzyme comprises one or more amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1, and wherein R is selected from H and a C₁-C₄ alkyl, preferably wherein R is methyl.

In some embodiments, there is provided a method for making a mixture comprising a compound of formula (I), wherein the method comprises contacting a mixture comprising a compound of formula (II) and a compound of formula (IIa) with a squalene-hopene cyclase (SHC) enzyme comprising an amino acid sequence having at least 70% identity or similarity with the sequence of SEQ ID NO: 1 or SEQ ID NOs: 43-49, preferably having at least 70% identity or similarity with the sequence of SEQ ID NO: 1 and comprising one or more amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1, and wherein R is selected from H and a C₁-C₄ alkyl, preferably wherein R is methyl. In some embodiments, the mixture comprising a compound of formula (I) further comprises a compound of formula (Ia), preferably having the configuration of a compound of formula (V), as described later herein.

As used herein, “contacting” may correspond to the physical interaction of a compound with a squalene-hopene cyclase (SHC) enzyme as described herein, which promotes the reaction catalyzed by the enzyme.

“Contacting with a compound of formula (II)” and “contacting with a compound of formula (IIa)” may correspond to contacting with a single isomer or with a mixture of isomers of these compounds. An “isomer” of a compound as used herein preferably refers to a stereoisomer of the compound.

An SHC enzyme may be produced in a host cell as described later herein. Such host cells may be used in the methods described herein. In some embodiments, an SHC enzyme may be associated with a membrane (such as a cell membrane or a membrane on which it is immobilized) in order to receive and/or interact with a substrate (e.g., a compound of formula (II) and/or a compound of formula (IIa)), which membrane (such as a cell membrane) can be part of a whole cell (e.g. a recombinant host cell, such as described later herein). An SHC enzyme may also be present in a crude cell extract or a cell-free extract. Accordingly, the skilled person understands that “contacting” may also correspond to the physical interaction of a compound with a cell expressing an SHC enzyme as described later herein, with a membrane fraction of said cell, with a crude cell extract of said cell, or with a cell-free extract of said cell. An SHC enzyme may also be in an immobilized form (e.g., associated with an enzyme carrier) which allows the SHC enzyme to interact with a substrate (e.g., a compound of formula (II) and/or a compound of formula (IIa)). A description of “immobilization” is provided later herein. An SHC enzyme may also be used in a soluble form.

### Compounds of Formulas (II) and (IIa)

A compound of formula (II), a compound of formula (IIa), as well as mixtures comprising them, may alternatively be referred to herein as “substrate”, “(bio) conversion substrate”, or “reaction substrate”, all terms being interchangeable. The numbering of carbon atoms in a compound of formula (II) is as follows:

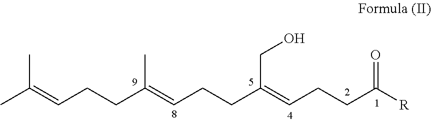

The numbering of carbon atoms in a compound of formula (IIa) is as follows:

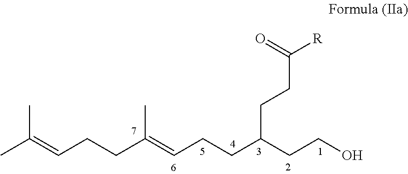

A compound of formula (IIa) is a “constitutional isomer” of a compound of formula (II). The SHC enzymes described herein are particularly suitable for converting a compound of formula (II) and/or a compound of formula (IIa) into useful products, as described later herein.

In embodiments comprising contacting with a mixture of isomers of a compound of formula (II), at least one isomer is converted to a compound of formula (I). In embodiments comprising contacting with a mixture of isomers of a compound of formula (IIa), at least one isomer is converted to a compound of formula (Ia). In embodiments comprising contacting with a mixture comprising a compound of formula (II) and a compound of formula (IIa), the compound of formula (II) may be converted to a compound of formula (I) and/or the compound of formula (IIa) may be converted to a compound of formula (Ia).

Compounds of formula (II) and (IIa) may occur in the form of four different isomers, for example, as a compound of formula (II) or a compound of formula (IIa) having an E,E-, Z,E-, Z,Z-, or E,Z-configuration, alternatively referred to herein as E,E-, Z,E-, Z,Z-, or E,Z-isomers. In some embodiments, the compound of formula (II) is such that the double bond between C-8 and C-9 is in E-configuration and the double bond between C-4 and C-5 is in Z-configuration (E,Z-isomer). In some embodiments, the compound of formula (II) is such that the double bond between C-8 and C-9 is in E-configuration and the double bond between C-4 and C-5 is in E-configuration (E,E-isomer).

A compound of formula (II) that has the double bond between C-8 and C-9 in Z-configuration and the double bond between C-4 and C-5 in E-configuration corresponds to the Z,E-isomer. A compound of formula (II) that has the double bond between C-8 and C-9 in Z-configuration and the double bond between C-4 and C-5 in Z-configuration corresponds to the Z,Z-isomer.

In some embodiments, the compound of formula (IIa) is such that the double bond between C-6 and C-7 is in E-configuration and the double bond between C-2 and C-3 is in Z-configuration (E,Z-isomer). In some embodiments, the compound of formula (IIa) is such that the double bond between C-6 and C-7 is in E-configuration and the double bond between C-2 and C-3 is in E-configuration (E,E-isomer).

A compound of formula (IIa) that has the double bond between C-6 and C-7 in Z-configuration and the double bond between C-2 and C-3 in E-configuration corresponds to the Z,E-isomer. A compound of formula (IIa) that has the double bond between C-6 and C-7 in Z-configuration and the double bond between C-2 and C-3 in Z-configuration corresponds to the Z,Z-isomer.

In some embodiments, the compound of formula (II) is a mixture of two or more than two of its isomers. In some embodiments, the mixture comprises an E,E-isomer and one or more other isomers of a compound of formula (II). In some embodiments, the mixture comprises an E,Z-isomer and one or more other isomers of a compound of formula (II). Accordingly, in some embodiments the mixture may comprise an E,E- and a Z,E-isomer. In some embodiments the mixture may comprise an E,E- and a Z,Z-isomer. In some embodiments the mixture may comprise an E,E- and a E,Z-isomer. In some embodiments the mixture may comprise an E,Z- and a Z,E-isomer. In some embodiments the mixture may comprise an E,Z- and a Z,Z-isomer.

In some embodiments, the compound of formula (IIa) is a mixture of two or more than two of its isomers. In some embodiments, the mixture comprises an E,E-isomer and one or more other isomers of a compound of formula (IIa). In some embodiments, the mixture comprises an E,Z-isomer and one or more other isomers of a compound of formula (IIa). Accordingly, in some embodiments the mixture may comprise an E,E- and a Z,E-isomer. In some embodiments the mixture may comprise an E,E- and a Z,Z-isomer. In some embodiments the mixture may comprise an E,E- and a E,Z-isomer. In some embodiments the mixture may comprise an E,Z- and a Z,E-isomer. In some embodiments the mixture may comprise an E,Z- and a Z,Z-isomer.

In some embodiments, the compound of formula (II) is a mixture of three or more than three of its isomers. In some embodiments, the mixture comprises an E,E-isomer and two or more other isomers of a compound of formula (II). In some embodiments, the mixture comprises an E,Z-isomer and two or more other isomers of a compound of formula (II). Accordingly, in some embodiments the mixture may comprise an E,E-, Z,E- and Z,Z-isomer. In some embodiments the mixture may comprise an E,E-, Z,E- and Z,Z-isomer. In some embodiments the mixture may comprise an E,E-, Z,E-, and E,Z-isomer. In some embodiments the mixture may comprise an Z,E-, Z,Z-, and E,Z-isomer.

In some embodiments, the compound of formula (IIa) is a mixture of three or more than three of its isomers. In some embodiments, the mixture comprises an E,E-isomer and two or more other isomers of a compound of formula (IIa). In some embodiments, the mixture comprises an E,Z-isomer and two or more other isomers of a compound of formula (IIa). Accordingly, in some embodiments the mixture may comprise an E,E-, Z,E- and Z,Z-isomer. In some embodiments the mixture may comprise an E,E-, Z,E- and Z,Z-isomer. In some embodiments the mixture may comprise an E,E-, Z,E-, and E,Z-isomer. In some embodiments the mixture may comprise an Z,E-, Z,Z-, and E,Z-isomer.

In some embodiments, the compound of formula (II) is a mixture comprising an E,Z-, E,E-, Z,E-, and a Z,Z-isomer. Preferred mixtures comprise an E,Z-isomer and/or an E,E-isomer of a compound of formula (II), preferably an E,Z-isomer.

In some embodiments, the compound of formula (IIa) is a mixture comprising an E,Z-, E,E-, Z,E-, and a Z,Z-isomer. Preferred mixtures comprise an E,Z-isomer and/or an E,E-isomer of a compound of formula (IIa), preferably an E,Z-isomer.

In some embodiments, a mixture comprises an E,Z-isomer of a compound of formula (II) and/or an E,E-isomer a compound of formula (II), preferably an E,Z-isomer of a compound of formula (II), and an E,Z-isomer a compound of formula (IIa) and/or an E,E-isomer of a compound of formula (IIa), preferably an E,Z-isomer of a compound of formula (IIa). Optionally, a Z,E-isomer of a compound of formula (II), a Z,Z-isomer of a compound of formula (II), a Z, E-isomer of a compound of formula (IIa), and/or a Z,Z-isomer of a compound of formula (IIa) may be comprised in the mixture.

In some embodiments, a method described herein comprises contacting an E,Z-isomer of a compound of formula (II) with a squalene-hopene cyclase (SHC) enzyme described herein. In some embodiments, a method described herein comprises contacting an E,Z-isomer and/or an E,E-isomer of a compound of formula (IIa), preferably an E,Z-isomer of a compound of formula (IIa), with a squalene-hopene cyclase (SHC) enzyme described herein.

In some embodiments, a method described herein comprises contacting a mixture comprising, consisting essentially of, or consisting of an E,E-isomer and an E,Z-isomer of a compound of formula (II) with a squalene-hopene cyclase (SHC) enzyme described herein. In some embodiments, the mixture comprises at least one of, or both, a Z, E-isomer and a Z,Z-isomer of a compound of formula (II). In some embodiments, the mixture does not comprise one of, or both, a Z,E-isomer and a Z,Z-isomer of a compound of formula (II).

In some embodiments, a method described herein comprises contacting a mixture comprising, consisting essentially of, or consisting of an E,E-isomer and an E,Z-isomer of a compound of formula (IIa) with a squalene-hopene cyclase (SHC) enzyme described herein. In some embodiments, the mixture comprises at least one of, or both, a Z,E-isomer and a Z,Z-isomer of a compound of formula (IIa). In some embodiments, the mixture does not comprise one of, or both, a Z,E-isomer and a Z,Z-isomer of a compound of formula (IIa).

In some embodiments, a method described herein comprises contacting a mixture comprising, consisting essentially of, or consisting of an E,E-isomer of a compound of formula (II) and an E,Z-isomer of a compound of formula (II) and/or an E,E-isomer of a compound of formula (IIa) and/or an E,Z-isomer of a compound of formula (IIa) with a squalene-hopene cyclase (SHC) enzyme described herein. In some embodiments, the mixture comprises at least one of, or both, a Z,E-isomer and a Z,Z-isomer of a compound of formula (II). In some embodiments, the mixture comprises at least one of, or both, a Z,E-isomer and a Z,Z-isomer of a compound of formula (IIa). In some embodiments, the mixture does not comprise one of, or both, a Z,E-isomer and a Z,Z-isomer of a compound of formula (II). In some embodiments, the mixture does not comprise one of, or both, a Z,E-isomer and a Z,Z-isomer of a compound of formula (IIa).

In a mixture comprising an E,Z-isomer of a compound of formula (II) and one or more other isomers of a compound of formula (II), the ratio of the E,Z-isomer to all other isomers combined may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60. In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 96:4 or about 96:4. In some embodiments, the ratio is equal to or greater than 97:3 or about 97:3. In some embodiments, the ratio is equal to or greater than 98:2 or about 98:2. In some embodiments, the ratio is equal to or greater than 99:1 or about 99:1.

In a mixture comprising an E,Z-isomer of a compound of formula (II) and one or more other isomers of a compound of formula (II), the ratio of the E,Z-isomer to all other isomers combined may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80.

In some embodiments, the ratio is equal to or lower than 10:90 or about 10:90.

In a mixture comprising an E,Z-isomer of a compound of formula (II) and one or more other isomers of a compound of formula (II), the ratio of the E,Z-isomer to all other isomers combined may range from 10:90 to 99:1, from 10:90 to 90:1, from 20:80 to 80:20, from 50:50 to 80:20, or from 60:40 to 80:20.

In a mixture comprising an E,Z-isomer of a compound of formula (IIa) and one or more other isomers of a compound of formula (IIa), the ratio of the E,Z-isomer to all other isomers combined may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60. In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In a mixture comprising an E,Z-isomer of a compound of formula (IIa) and one or more other isomers of a compound of formula (IIa), the ratio of the E,Z-isomer to all other isomers combined may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80. In some embodiments, the ratio is equal to or lower than 10:90 or about 10:90.

In a mixture comprising an E,Z-isomer of a compound of formula (IIa) and one or more other isomers of a compound of formula (IIa), the ratio of the E,Z-isomer to all other isomers combined may range from 10:90 to 99:1, from 10:90 to 90:1, from 20:80 to 80:20, from 50:50 to 80:20, or from 60:40 to 80:20.

In a mixture comprising an E,Z-isomer and an E, E-isomer of a compound of formula (II), the ratio of the E,Z-isomer to the E, E-isomer may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60. In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In a mixture comprising an E,Z-isomer and an E, E-isomer of a compound of formula (II), the ratio of the E,Z-isomer to the E,E-isomer may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80. In some embodiments, the ratio is equal to or lower than 10:90 or about 10:90.

In a mixture comprising an E,Z-isomer and an E, E-isomer of a compound of formula (II), the ratio of the E,Z-isomer to the E,E-isomer may be from 10:90 to 99:1 or from about 10:90 to about 99:1, from 10:90 to 90:1 or from about 10:90 to about 90:1, from 20:80 to 80:20 or from about 20:80 to about 80:20, from 50:50 to 80:20 or from about 50:50 to about 80:20, or from 60:40 to 80:20 or from about 60:40 to about 80:20.

In a mixture comprising an E,Z-isomer and an E,E-isomer of a compound of formula (IIa), the ratio of the E,Z-isomer to the E,E-isomer may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60. In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In a mixture comprising an E,Z-isomer and an E,E-isomer of a compound of formula (IIa), the ratio of the E,Z-isomer to the E,E-isomer may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80. In some embodiments, the ratio is equal to or lower than 10:90 or about 10:90.

In a mixture comprising an E,Z-isomer and an E,E-isomer of a compound of formula (IIa), the ratio of the E,Z-isomer to the E,E-isomer may be from 10:90 to 99:1 or from about 10:90 to about 99:1, from 10:90 to 90:1 or from about 10:90 to about 90:1, from 20:80 to 80:20 or from about 20:80 to about 80:20, from 50:50 to 80:20 or from about 50:50 to about 80:20, or from 60:40 to 80:20 or from about 60:40 to about 80:20.

In a mixture comprising an E,Z-isomer of a compound of formula (II) and an E,Z-isomer of a compound of formula (IIa), the ratio of the E,Z-isomer of a compound of formula (II) to the E,Z-isomer of a compound of formula (IIa) may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60.

In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In a mixture comprising an E,Z-isomer of a compound of formula (II) and an E,Z-isomer of a compound of formula (IIa), the ratio of the E,Z-isomer of a compound of formula (II) to the E,Z-isomer of a compound of formula (IIa) may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80. In some embodiments, the ratio is equal to or lower than 10:90 or about 10:90.

In a mixture comprising an E,Z-isomer of a compound of formula (II) and an E,Z-isomer of a compound of formula (IIa), the ratio of the E,Z-isomer of a compound of formula (II) to the E,Z-isomer of a compound of formula (IIa) may be from 10:90 to 99:1, from 10:90 to 90:1, from 20:80 to 80:20, from 50:50 to 80:20, or from 60:40 to 80:20.

In a mixture comprising an E,Z-isomer of a compound of formula (II) and an E,E-isomer of a compound of formula (IIa), the ratio of the E,Z-isomer of a compound of formula (II) to the E,E-isomer of a compound of formula (IIa) may be equal to or greater than 10:90 or about 10:90. In some embodiments, the ratio is equal to or greater than 20:80 or about 20:80. In some embodiments, the ratio is equal to or greater than 30:70 or about 30:70. In some embodiments, the ratio is equal to or greater than 40:60 or about 40:60. In some embodiments, the ratio is equal to or greater than 50:50 or about 50:50. In some embodiments, the ratio is equal to or greater than 60:40 or about 60:40. In some embodiments, the ratio is equal to or greater than 70:30 or about 70:30. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In a mixture comprising an E,Z-isomer of a compound of formula (II) and an E,E-isomer of a compound of formula (IIa), the ratio of the E,Z-isomer of a compound of formula (II) to the E,E-isomer of a compound of formula (IIa) may be equal to or lower than 99:1 or about 99:1. In some embodiments, the ratio is equal to or lower than 95:5 or about 95:5. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 70:30 or about 70:30. In some embodiments, the ratio is equal to or lower than 60:40 or about 60:40. In some embodiments, the ratio is equal to or lower than 50:50 or about 50:50. In some embodiments, the ratio is equal to or lower than 40:60 or about 40:60. In some embodiments, the ratio is equal to or lower than 30:70 or about 30:70. In some embodiments, the ratio is equal to or lower than 20:80 or about 20:80. In some embodiments, the ratio is equal to or lower than 10:90 or about 10:90.

In a mixture comprising an E,Z-isomer of a compound of formula (II) and an E,E-isomer of a compound of formula (IIa), the ratio of the E,Z-isomer of a compound of formula (II) to the E,E-isomer of a compound of formula (IIa) may be from 10:90 to 99:1, from 10:90 to 90:1, from 20:80 to 80:20, from 50:50 to 80:20, or from 60:40 to 80:20.

The skilled person understands that the ratios discussed above may, for example, be determined by dividing steroisomer weights or concentrations.

The ratio of a given isomer to one or more other isomers in a mixture of isomers may be quantified using routine methods available to the skilled person, such as gas chromatography, optionally in combination with mass spectrometry, and nuclear magnetic resonance (NMR) spectroscopy, examples of which may be found in standard handbooks in the art such as Encyclopedia of Analytical Science: 3^{rd} Edition, Eds. Paul Worsfold, Alan Townshend, Colin Poole, Manuel Miro, Elsevier (2019), incorporated herein by reference in its entirety. The skilled person understands that these methods may also be used to quantify the concentration of an isomer in a mixture, such as, for example, an aqueous solution. Concentration of an isomer in a mixture may be expressed using multiple quantitative units, examples being molarity, molality, mass percentage, parts per thousand (ppth), parts per million (ppm), and parts per billion (ppb). Interconversion of these units as well as calculation of isomer weight in a given mixture based on concentration values are all well within the capabilities of the skilled person.

In some embodiments, R is selected from H (hydrogen) and a C₁-C₄ alkyl such as methyl, ethyl, n-propyl, or isopropyl. Preferably, R is methyl. A compound of formula (II) wherein R is methyl may be referred to as hydroxyfarnesylacetone (HFA), encompassing the respective compounds E,E-hydroxyfarnesylacetone (E,E-HFA), Z,E-hydroxyfarnesylacetone (Z,E-HFA), Z,Z-hydroxyfarnesylacetone (Z,Z-HFA), and E,Z-hydroxyfarnesylacetone (E,Z-HFA), as well as mixtures thereof. Among the isomers of hydroxyfarnesylacetone, E,Z-hydroxyfarnesylacetone is preferred.

Among the isomers of a compound of formula (IIa), the E,Z-isomer and the E,E-isomers are preferred, with the E,Z-isomer being further preferred.

Accordingly, in some embodiments, a mixture comprising a compound of formula (II) and a compound of formula (IIa) comprises any one of the following:
i) a compound of formula (II) that is such that the double bond between C-8 and C-9 is in E-configuration and the double bond between C-4 and C-5 is in Z-configuration (E,Z-isomer)
ii) a compound of formula (II) that is such that the double bond between C-8 and C-9 is in E-configuration and the double bond between C-4 and C-5 is in E-configuration (E,E-isomer)
iii) a compound of formula (IIa) that is such that the double bond between C-6 and C-7 is in E-configuration and the double bond between C-2 and C-3 is in Z-configuration (E,Z-isomer)
iv) a compound of formula (IIa) that is such that the double bond between C-6 and C-7 is in E-configuration and the double bond between C-2 and C-3 is in E-configuration (E,E-isomer)
v) a compound of formula (II) that is such that the double bond between C-8 and C-9 is in E-configuration and the double bond between C-4 and C-5 is in Z-configuration (E,Z-isomer) and a compound of formula (II) that is such that the double bond between C-8 and C-9 is in E-configuration and the double bond between C-4 and C-5 is in E-configuration (E,E-isomer)
vi) a compound of formula (IIa) that is such that the double bond between C-6 and C-7 is in E-configuration and the double bond between C-2 and C-3 is in Z-configuration (E,Z-isomer) and a compound of formula (IIa) that is such that the double bond between C-6 and C-7 is in E-configuration and the double bond between C-2 and C-3 is in E-configuration (E,E-isomer)
vii) any combination of i)-vi)

In some embodiments, a mixture comprising a compound of formula (II) and a compound of formula (IIa) comprises:
a compound of formula (II) that is such that the double bond between C-8 and C-9 is in E-configuration and the double bond between C-4 and C-5 is in Z-configuration (E,Z-isomer)
a compound of formula (II) that is such that the double bond between C-8 and C-9 is in E-configuration and the double bond between C-4 and C-5 is in E-configuration (E,E-isomer)
a compound of formula (IIa) that is such that the double bond between C-6 and C-7 is in E-configuration and the double bond between C-2 and C-3 is in Z-configuration (E,Z-isomer), and;
a compound of formula (IIa) that is such that the double bond between C-6 and C-7 is in E-configuration and the double bond between C-2 and C-3 is in E-configuration (E,E-isomer).

Such a mixture may optionally comprise the isomers of a compound of formula (II) and of a compound of formula (IIa) in a specific E,Z-isomer of a compound of formula (II): E,E-isomer of a compound of formula (II): E,Z-isomer of a compound of formula (IIa): E,E-isomer of a compound of formula (IIa) ratio, such as, but not limited to, 37:9:29:16 or about 37:9:29:16, or 27:36:13:24 or about 27:36:13:24. Optionally, the mixture comprises a Z,E-isomer of a compound of formula (II), a Z,Z-isomer of a compound of formula (II), a Z,E-isomer of a compound of formula (IIa), and/or a Z,Z-isomer of a compound of formula (IIa).

The skilled person understands that, in the context of the disclosure, following the “contacting with a compound of formula (II)”, it is not necessary that all of the compound will be converted to a compound of formula (I). Similarly, following the “contacting with a compound of formula (IIa)”, it is not necessary that all of the compound will be converted to a compound of formula (Ia). As an example, a reaction by-product may be formed (for example the ones described later herein), or the compound of formula (II) and/or the compound of formula (IIa) may not be completely converted. As another example, in a mixture comprising two or more isomers of a compound of formula (II), not all isomers are necessarily converted to a compound of formula (I). As another example, in a mixture comprising two or more isomers of a compound of formula (IIa), not all isomers are necessarily converted to a compound of formula (Ia). As another example, in a mixture comprising a compound of formula (II) and a compound of formula (IIa), not all of compound of formula (II) is necessarily converted to a compound of formula (I) and/or not all of compound of formula (IIa) is necessarily converted to a compound of formula (Ia).

In some embodiments, not all of the compound of formula (II) is converted to a compound of formula (I) or a reaction by-product, resulting in a product, such as a composition, comprising a compound of formula (II) and a compound of formula (I). In some embodiments, any non-converted compound of formula (II) in the product, such as a composition, may be isolated and/or purified from the product such that a product that does not comprise any compound of formula (II) is obtained. In some embodiments, all of the compound of formula (II) is converted to a compound of formula (I) or a reaction by-product.

In some embodiments, not all of the compound of formula (IIa) is converted to a compound of formula (Ia) or a reaction by-product, resulting in a product, such as a composition, comprising a compound of formula (IIa) and a compound of formula (Ia). In some embodiments, any non-converted compound of formula (IIa) in the product, such as a composition, may be isolated and/or purified from the product such that a product that does not comprise any compound of formula (IIa) is obtained. In some embodiments, all of the compound of formula (IIa) is converted to a compound of formula (Ia) or a reaction by-product.

In some embodiments, in a mixture comprising a compound of formula (II) and a compound of formula (IIa), not all of the compound of formula (II) is converted to a compound of formula (I) or a reaction by-product and/or not all of the compound of formula (IIa) is converted to a compound of formula (Ia) or a reaction by-product. In some embodiments, any non-converted compound of formula (II) and/or of compound of formula (IIa) in the product, such as a composition, may be isolated and/or purified from the product such that a product that does not comprise any compound of formula (II) and/or a compound of formula (IIa) is obtained. In some embodiments, all of the compound of formula (II) is converted to a compound of formula (I) or a reaction by-product. In some embodiments, all of the compound of formula (IIa) is converted to a compound of formula (Ia) or a reaction by-product.

Isolation and/or purification are discussed later herein.

In embodiments wherein a compound of formula (II) and/or a compound of formula (IIa) corresponds to a mixture of isomers, the presence of the various isomers may influence the conversion; for example, the reaction rate may be decreased.

Thus, an SHC enzyme described herein may be capable of converting an E,Z-isomer of a compound of formula (II) to a compound of formula (I) from a mixture of isomers of a compound of formula (II). An SHC enzyme described herein may be capable of converting an E,Z-isomer of a compound of formula (IIa) to a compound of formula (Ia) from a mixture of isomers of a compound of formula (IIa).

An SHC enzyme described herein may be capable of converting an E,Z-isomer of a compound of formula (II) to a compound of formula (I) from a mixture comprising isomers of a compound of formula (II) and of a compound of formula (IIa).

An SHC enzyme described herein may be capable of converting an E,Z-isomer of a compound of formula (IIa) to a compound of formula (Ia) from a mixture comprising isomers of a compound of formula (IIa) and of a compound of formula (II).

A mixture may comprise two of the isomers of a compound of formula (II), for example the E,Z-isomer and the E,E-isomer. The mixture may comprise three of the isomers of a compound of formula (II), for example the E,Z-isomer, the E,E-isomer, and one of the Z,E-isomer or the Z,Z-isomer. The mixture may comprise four isomers of a compound of formula (II), i.e., the E,Z-isomer, the E,E-isomer, the Z,E-isomer, and the Z,Z-isomer. The presence of other isomers of a compound of formula (II) may decrease the conversion rate of the E,Z-isomer to a compound of formula (I). Without wishing to be bound by theory, a possible explanation can be that the other isomers may compete with the E,Z-isomer of formula (II) for access to the SHC enzyme and thus may act as competitive inhibitors for the conversion of the E,Z-isomer of a compound of formula (II) to a compound of formula (I), and/or act as alternative substrates. Accordingly, a reaction substrate may refer to an isomeric mixture of 2-4 isomers of a compound of formula (II), preferably two isomers. In some embodiments, a reaction substrate comprises, consists essentially of, or consists of an isomeric mixture of an E,Z-isomer and an E,E-isomer of a compound of formula (II).

A mixture may comprise two of the isomers of a compound of formula (IIa), for example the E,Z-isomer and the E, E-isomer. The mixture may comprise three of the isomers of a compound of formula (IIa), for example the E,Z-isomer, the E, E-isomer, and one of the Z, E-isomer or the Z,Z-isomer. The mixture may comprise four isomers of a compound of formula (IIa), i.e., the E,Z-isomer, the E,E-isomer, the Z,E-isomer, and the Z,Z-isomer. Accordingly, a reaction substrate may refer to an isomeric mixture of 2-4 isomers of a compound of formula (IIa), preferably two isomers. In some embodiments, a reaction substrate comprises, consists essentially of, or consists of an isomeric mixture of an E,Z-isomer and an E,E-isomer of a compound of formula (IIa).

A mixture may comprise two of the isomers of a compound of formula (II), for example the E,Z-isomer and the E, E-isomer, and two of the isomers of a compound of formula (IIa), for example the E,Z-isomer and the E, E-isomer. The mixture may comprise three of the isomers of a compound of formula (II), for example the E,Z-isomer, the E,E-isomer, and one of the Z, E-isomer or the Z,Z-isomer and three of the isomers of a compound of formula (IIa), for example the E,Z-isomer, the E,E-isomer, and one of the Z,E-isomer or the Z,Z-isomer. The mixture may comprise four isomers of a compound of formula (II), i.e., the E,Z-isomer, the E,E-isomer, the Z,E-isomer, and the Z,Z-isomer and four isomers of a compound of formula (IIa), i.e., the E,Z-isomer, the E,E-isomer, the Z, E-isomer, and the Z,Z-isomer.

Accordingly, a reaction substrate may refer to an isomeric mixture of 2-4 isomers of a compound of formula (II), preferably two isomers, and of 2-4 isomers of a compound of formula (IIa), preferably two isomers.

In some embodiments, a reaction substrate comprises, consists essentially of, or consists of an isomeric mixture of an E,Z-isomer of a compound of formula (II), an E,E-isomer of a compound of formula (II), an E,Z-isomer of a compound of formula (IIa), and an E,E-isomer of a compound of formula (IIa).

A compound of formula (II) and a compound of formula (IIa) may be synthesized following the general procedure depicted by Fujiwara et al. (Tetrahedron Letters, 1995 Vol 36 (46), 8435-8438), incorporated herein by reference in its entirety. An additional general procedure is described in GB 2108985.9, incorporated herein by reference in its entirety.

Alternatively, a compound of formula (II) may be obtained as briefly demonstrated in FIG. 1, optionally wherein R is selected from H (hydrogen) and a C₁-C₄ alkyl, such as methyl, ethyl, n-propyl, or isopropyl.

### Compounds of Formulas (I) and (La)

As used herein, “making a compound of formula (I)” and “making a compound of formula (Ia)” may be also be referred to as “producing” or “obtaining” the respective compound. It may also refer to “producing” or “obtaining” a mixture comprising, consisting essentially of, or consisting of the respective compound.

Compounds of formula (I) and (la) comprise a number of chiral carbon atoms. Thus, one or more isomers of a compound of formula (I) and of formula (Ia) may occur, such as, for example, enantiomers and diastereomers. In addition to the compound of formula (I), the products made by the methods described herein may comprise one or more other isomers of a compound of formula (I). In addition to the compound of formula (Ia), the products made by the methods described herein may comprise one or more other isomers of a compound of formula (Ia). In this context, these other isomers may represent by-products of the enzymatic conversion. The isomers obtained by the methods described herein may depend on the isomers of a compound of formula (II) and/or of a compound of formula (IIa) that an SHC enzyme as described herein is contacted with.

As a non-limiting example, contacting a compound of formula (II) with an SHC enzyme as described herein may result in a compound of formula (IV) being made:

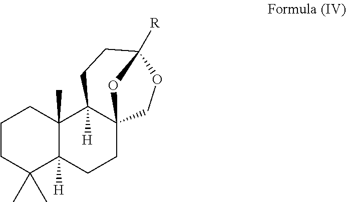

In some embodiments, R is selected from H (hydrogen) and a C₁-C₄ alkyl such as methyl, ethyl, n-propyl, or isopropyl, preferably wherein R is methyl.

A compound of formula (IV) wherein R is methyl is also known as (−)-epi-8-amberketal. A compound of formula (I), wherein R is methyl is also known as (+)-amberketal. Accordingly, in some embodiments, a compound of formula (I) and one or more other isomers of a compound of formula (I) are made such as, but not limited to, a compound of formula (IV), optionally wherein R is selected from H (hydrogen) and a C₁-C₄ alkyl such as methyl, ethyl, n-propyl, or isopropyl. Thus a product, such as the compositions described later herein, may comprise a compound of formula (I) and optionally one or more other isomers of a compound of formula (I) such as, but not limited to, a compound of formula (IV), optionally wherein R is selected from H (hydrogen) and a C₁-C₄ alkyl such as methyl, ethyl, n-propyl, or isopropyl.

A preferred compound of formula (Ia) has the configuration of formula (V):

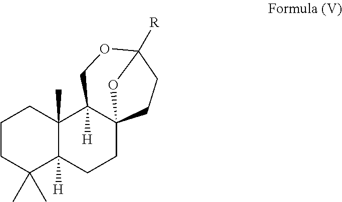

In some embodiments, R is selected from H (hydrogen) and a C₁-C₄alkyl, such as methyl, ethyl, n-propyl, or isopropyl, preferably R is methyl.

Accordingly, in some embodiments, a method described herein results in a compound of formula (V) being made. Thus a product, such as the compositions described later herein, may comprise a compound of formula (V) and optionally one or more other isomers of a compound of formula (Ia), optionally wherein R is selected from H (hydrogen) and a C₁-C₄ alkyl, such as methyl, ethyl, n-propyl, or isopropyl.

In some embodiments, a method described herein results in a product, such as the compositions described later herein, which may comprise a compound of formula (I) and a compound of formula (V), optionally wherein R is selected from H (hydrogen) and a C₁-C₄ alkyl, such as methyl, ethyl, n-propyl, or isopropyl. Optionally, the product may comprise one or more other isomers of a compound of formula (I), such as, but not limited to, a compound of formula (IV), and/or one or more other isomers of a compound of formula (Ia).

In some embodiments, the ratio of a compound of formula (I) to all other isomers of a compound of formula (I) combined, made by a method or comprised in a product, such as a composition, as described herein, is equal to or greater than 50:50 or about 50:50. In some embodiments, the ratio is equal to or greater than 55:45 or about 55:45. In some embodiments, the ratio is equal to or greater than 60:40 or about 60:40. In some embodiments, the ratio is equal to or greater than 65:35 or about 65:35. In some embodiments, the ratio is equal to or greater than 70:30 or about 70:30. In some embodiments, the ratio is equal to or greater than 75:25 or about 75:25. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In some embodiments, the ratio of a compound of formula (V) to all other isomers of a compound of formula (Ia) combined, made by a method or comprised in a product, such as a composition, as described herein, is equal to or greater than 50:50 or about 50:50. In some embodiments, the ratio is equal to or greater than 55:45 or about 55:45. In some embodiments, the ratio is equal to or greater than 60:40 or about 60:40. In some embodiments, the ratio is equal to or greater than 65:35 or about 65:35. In some embodiments, the ratio is equal to or greater than 70:30 or about 70:30. In some embodiments, the ratio is equal to or greater than 75:25 or about 75:25. In some embodiments, the ratio is equal to or greater than 80:20 or about 80:20. In some embodiments, the ratio is equal to or greater than 85:15 or about 85:15. In some embodiments, the ratio is equal to or greater than 90:10 or about 90:10. In some embodiments, the ratio is equal to or greater than 95:5 or about 95:5. In some embodiments, the ratio is equal to or greater than 99:1 or about 99:1.

In some embodiments, only a compound of formula (I) and no other isomers of a compound of formula (I) are made by the methods described herein, for example no compound of formula (IV), optionally wherein R is selected from H (hydrogen) and a C₁-C₄ alkyl, such as methyl, ethyl, n-propyl, or isopropyl. In some embodiments, only a compound of formula (V) and no other isomers of a compound of formula (Ia) are made by the methods described herein, optionally wherein R is selected from H (hydrogen) and a C₁-C₄ alkyl, such as methyl, ethyl, n-propyl, or isopropyl.

In some embodiments, any isomer other than a compound of formula (I) and/or a compound of formula (V) may be separated from a product, such as a composition, made by a method described herein, such that a product that does not comprise any other isomers is obtained; for example, a compound of formula (IV), optionally wherein R is H (hydrogen), methyl, or ethyl, is separated from and no longer present in the product. In other words, a composition as described herein may, for example, comprise 100 wt % of a compound of formula (I) and no other isomers of this compound (alternatively referred to herein as a 100:0 ratio). Similarly, a composition as described herein may, for example, comprise 100 wt % of a compound of formula (V) and no other isomers of a compound of formula (Ia). A composition as described herein may, for example, be a mixture comprising, consisting essentially of, or consisting of, preferably comprising, a compound of formula (I) and a compound of formula (V). Separation methods are known to the skilled person and discussed earlier herein.

In some embodiments, the ratio of a compound of formula (I) to all other isomers of a compound of formula (I) combined, made by a method or comprised in a product, such as a composition, as described herein, is equal to or lower than 99:1 or about 99:1. In some embodiments, the ratio is equal to or lower than 98:2 or about 98:2. In some embodiments, the ratio is equal to or lower than 97:3 or about 97:3. In some embodiments, the ratio is equal to or lower than 96:4 or about 96:4. In some embodiments, the ratio is equal to or lower than 95:5 or about 95:5.

In some embodiments, the ratio of a compound of formula (I) to all other isomers of a compound of formula (I) combined, made by a method or comprised in a product, such as a composition, as described herein, may be from 50:50 to 100:0 or from about 50:50 to about 100:0, from 60:40 to 99:1 or from about 60:40 to about 99:1, from 70:30 to 98:2 or from about 70:30 to about 98:2, from 80:20 to 97:3 or from about 80:20 to about 97:3, or from 90:10 to 97:3 or from about 90:10 to about 97:3.

In some embodiments, the ratio of a compound of formula (V) to all other isomers of a compound of formula (Ia) combined, made by a method or comprised in a product, such as a composition, as described herein, is equal to or lower than 99:1 or about 99:1. In some embodiments, the ratio is equal to or lower than 98:2 or about 98:2. In some embodiments, the ratio is equal to or lower than 97:3 or about 97:3. In some embodiments, the ratio is equal to or lower than 96:4 or about 96:4. In some embodiments, the ratio is equal to or lower than 95:5 or about 95:5.

In some embodiments, the ratio of a compound of formula (V) to all other isomers of a compound of formula (Ia) combined, made by a method or comprised in a product, such as a composition, as described herein, may be from 50:50 to 100:0 or from about 50:50 to about 100:0, from 60:40 to 99:1 or from about 60:40 to about 99:1, from 70:30 to 98:2 or from about 70:30 to about 98:2, from 80:20 to 97:3 or from about 80:20 to about 97:3, or from 90:10 to 97:3 or from about 90:10 to about 97:3.

In some embodiments, the ratio of a compound of formula (I) to a compound of formula (Ia) (such as a compound of formula (V)) made by a method or comprised in a product, such as a composition, as described herein, is equal to or lower than 99:1 or about 99:1. In some embodiments, the ratio is equal to or lower than 98:2 or about 98:2. In some embodiments, the ratio is equal to or lower than 97:3 or about 97:3. In some embodiments, the ratio is equal to or lower than 96:4 or about 96:4. In some embodiments, the ratio is equal to or lower than 95:5 or about 95:5. In some embodiments, the ratio is equal to or lower than 94:6 or about 94:6. In some embodiments, the ratio is equal to or lower than 93:7 or about 93:7. In some embodiments, the ratio is equal to or lower than 92:8 or about 92:8. In some embodiments, the ratio is equal to or lower than 91:9 or about 91:9. In some embodiments, the ratio is equal to or lower than 90:10 or about 90:10. In some embodiments, the ratio is equal to or lower than 85:15 or about 85:15. In some embodiments, the ratio is equal to or lower than 80:20 or about 80:20. In some embodiments, the ratio is equal to or lower than 75:25 or about 75:25. In some embodiments, the ratio is equal to or lower than 70:30 or about 70:30. In some embodiments, the ratio is equal to or lower than 65:35 or about 65:35. In some embodiments, the ratio is equal to or lower than 60:40 or about 60:40. In some embodiments, the ratio is equal to or lower than 55:45 or about 55:45. In some embodiments, the ratio is equal to or lower than 50:50 or about 50:50. In some embodiments, the ratio is equal to or lower than 49:51 or about 49:51. In some embodiments, the ratio is equal to or lower than 49:51 or about 49:51. In some embodiments, the ratio is equal to or lower than 48:52 or about 48:52. In some embodiments, the ratio is equal to or lower than 47:53 or about 47:53. In some embodiments, the ratio is equal to or lower than 46:54 or about 46:54. In some embodiments, the ratio is equal to or lower than 45:55 or about 45:55. In some embodiments, the ratio is equal to or lower than 44:56 or about 44:56. In some embodiments, the ratio is equal to or lower than 43:57 or about 43:57. In some embodiments, the ratio is equal to or lower than 42:58 or about 42:58. In some embodiments, the ratio is equal to or lower than 41:59 or about 41:59. In some embodiments, the ratio is equal to or lower than 40:60 or about 40:60.

In some embodiments, the ratio of a compound of formula (I) to a compound of formula (Ia) (such as a compound of formula (V)) made by a method or comprised in a product, such as a composition, as described herein, may be from 40:60 to 100:0 or from about 40:60 to about 100:0, from 60:40 to 99:1 or from about 60:40 to about 99:1, from 70:30 to 98:2 or from about 70:30 to about 98:2, from 80:20 to 97:3 or from about 80:20 to about 97:3, or from 90:10 to 97:3 or from about 90:10 to about 97:3, or from 93:7 to 97:3 or from about 97:3 to about 97:3.

The ratio of a given isomer of a compound of formula (I) and/or of a compound of formula (Ia) (such as a compound of formula (V)) to one or more other isomers of the respective compound in a mixture of isomers, as well as amounts and concentrations of isomers, may be determined as discussed earlier herein, using routine methods available to the skilled person, such as gas chromatography (optionally on chiral columns), or NMR spectroscopy (optionally in the presense of shift reagents), which are available to the skilled person. The same methods can be used to determine the ratio of a given isomer of a compound of formula (I) to a compound of formula (V) and/or to another isomer of a compound of formula (Ia).

A compound of formula (I), and/or a compound of formula (Ia) (such as a compound of formula (V)) made by the methods described herein may, for example, be comprised in a mixture. A compound of formula (I), and/or a compound of formula (Ia) (such as a compound of formula (V)) made by the methods described herein may, for example, be in a solid form, preferably in an amorphous or crystalline form. A compound of formula (I), and/or a compound of formula (Ia) (such as a compound of formula (V)) made by the methods described herein may, for example be in the solid phase in a reaction mixture.

Such a form may be advantageous, as the presence of a compound in a solid form/the solid phase can simplify downstream processing after the compound is made. As a non-limiting example, when host cells expressing the SHC enzymes as described herein are used a biocatalyst, and the compound of formula (I) and/or compound of formula (Ia) (such as a compound of formula (V)) are made in a solid form (such as an amorphous or crystalline form), the compounds may be easily separated from the reaction mixture (which may also correspond to a cell culture as described later herein) via simple techniques such as filtration and/or centrifugation. Optionally, the obtained compound of formula (I) and/or compound of formula (Ia) (such as compound of formula (V)) may be further isolated and/or purified as described herein, in any case requiring fewer materials (e.g., solvents) and/or less energy input relative to cases wherein the compound of formula (I) and/or compound of formula (Ia) (such as compound of formula (V)) are not made in a solid form (such as an amorphous or crystalline form).

A compound of formula (I) and/or a compound of formula (Ia) (such as a compound of formula (V)), may be isolated and/or purified after it is made. Accordingly, in some embodiments, a compound of formula (I) and/or a compound of formula (Ia) (such as a compound of formula (V)), is isolated. Optionally, a compound of formula (I) and/or a compound of formula (Ia) (such as a compound of formula (V)), is purified. The term “isolation” as used herein refers to separation (alternatively referred to herein as “extraction”) of a compound, such as a compound of formula (I) and/or a compound of formula (Ia) (such as a compound of formula (V)), from components which accompany it. The degree of isolation or purity of a compound can be measured by any method commonly used in the art, e.g., gas chromatography (GC), chromatographic methods (e.g., HPLC) or NMR spectroscopy, which are all known to the skilled person and are summarized in standard handbooks, such as the Encyclopedia of Analytical Science: 3^{rd} Edition (supra).

Isolation may be accomplished by any method commonly used in the art. Examples of suitable methods include steam extraction, distillation, or organic solvent extraction using a non-water miscible solvent (which separates the reaction products and unreacted substrates from the biocatalyst that stays in the aqueous phase) followed by subsequent evaporation of the solvent to obtain a crude reaction product as determined by gas chromatography analysis. These methods are known to the skilled person and are summarized in standard handbooks, such as the Encyclopedia of Analytical Science: 3rd Edition (supra).

By way of example, a produced compound of formula (I) and/or a compound of formula (Ia) (such as a compound of formula (V)) may be extracted from the whole reaction mixture using an organic solvent such as a non-water miscible solvent (for example toluene). Alternatively, a produced compound of formula (I) and/or a compound of formula (Ia) (such as a compound of formula (V)) may be extracted from the solid phase of the reaction mixture (obtained by, for example, centrifugation or filtration) using a water miscible solvent (for example ethanol) or a non-water miscible solvent (for example toluene). By way of further example, a compound of formula (I) and/or a compound of formula (Ia) (such as a compound of formula (V)) may be present in the solid phase as crystals or in amorphous form, as discussed earlier herein, and may be separated from the remaining solid phase (cell material or debris thereof) and the liquid phase also by means of filtration. By way of further example, at a temperature above the melting point of the compound of formula (I) and/or a compound of formula (Ia) (such as a compound of formula (V)), a compound of formula (I) and/or a compound of formula (Ia) (such as a compound of formula (V)) may form an oil layer on top of aqueous phase, which oil layer can be removed and collected. In order to ensure a complete recovery of the compound after the oil layer is removed, an organic solvent may be added to the aqueous phase containing the biomass in order to extract any residual compound of formula (I) (e.g., (+)-amberketal) and/or a compound of formula (Ia) (such as a compound of formula (V)) contained in, or on or about the biomass. The organic layer can be combined with the oil layer, before the whole is further processed to isolate and purify the compound of formula (I) and/or a compound of formula (Ia) (such as a compound of formula (V)). The compound of formula (I) and/or a compound of formula (Ia) (such as a compound of formula (V)) may be further selectively crystallised to remove by-products and any unreacted compound of formula (II) and/or a compound of formula (IIa) from the final product.

Purification may be accomplished by any method commonly used in the art, which are known to the skilled and are summarized in standard handbooks, such as the Encyclopedia of Analytical Science: 3^{rd} Edition (supra). Further examples of isolation and purification are provided in the experimental section herein.

The term “selective crystallization” refers to a process step whereby a compound of formula (I) and/or a compound of formula (Ia) (such as a compound of formula (V)) is caused to crystallise from a solvent whilst the by-products remain dissolved in the crystallising solvent to such an extent that isolated crystalline material contains only the compound of formula (I) and/or a compound of formula (Ia) (such as a compound of formula (V)), or if it contains any byproducts, then they are present only in olfactory acceptable amounts. The compound of formula (I), for example, is free or substantially free of by-products such as a compound of formula (III) or (IIIa) (described later herein). The compound of formula (Ia), preferably the compound of formula (V), for example, is free or substantially free of by-products such as a compound of formula (VI) or (VIa) (described later herein). The selective crystallisation step may use a water miscible solvent such as ethanol or the like. The selective crystallisation of a compound of formula (I) and/or a compound of formula (Ia) (such as a compound of formula (V)) may be influenced by the presence of unreacted compound of formula (II) and/or unreacted compound of formula (IIa) and also the ratio of compound of formula (I) and/or of formula (Ia) (such as of formula (V)) to the other detectable byproducts. Even if only 10% conversion of a compound of formula (II) to a compound of formula (I) is obtained, the selective crystallization of the produced compound may still be possible. Similarly, even if only 10% conversion of a compound of formula (IIa) to a compound of formula (Ia), preferably to a compound of formula (V) is obtained, the selective crystallization of the produced compound may still be possible.

The purity of the final compound of formula (I) and/or of the final compound of formula (Ia) (such as a compound of formula (V)) obtained can be determined using routine gas chromatography (GC) techniques. Similar techniques can also be applied to mixtures comprising a compound of formula (I) and a compound of formula (Ia) (such as a compound of formula (V)).

The olfactive purity of a product comprising a compound of formula (I), a compound of formula (Ia) (such as a compound of formula (V)), or a mixture comprising a compound of formula (I) and a compound of formula (Ia) (such as a compound of formula (V)) product may be determined by testing the crystalline material or a solution of the crystalline material in ethanol. The product comprising a compound of formula (I), a compound of formula (Ia) (such as a compound of formula (V)), or a mixture comprising a compound of formula (I) and a compound of formula (Ia) (such as a compound of formula (V)) may be tested against a commercially available reference of a compound of formula (I), a commercially available reference of a compound of formula (Ia) (such as of a compound of formula (V)), or a commercially available reference mixture comprising a compound of formula (I) and a compound of formula (Ia) (such as a compound of formula (V)) for its olfactive purity, quality and its sensory profile by a trained olfactory expert or a trained olfactory expert panel. The product may also be tested in application studies by trained olfactory experts in order to determine whether the material meets the specifications with respect to its olfactive profile thus providing an olfactively acceptable product.

The term “olfactively pure” as it is used in relation to a product of the disclosure, is intended to mean that a compound of formula (I), a compound of formula (Ia) (such as a compound of formula (V)), or a mixture comprising a compound of formula (I) and a compound of formula (Ia) (such as a compound of formula (V)) product is free of compounds (II), (IIa), (III), (IIIa), (IV), (IVa), (V), (Va), (VI), and/or (Via) and/or any other material found in the reaction mixture, or that if such compounds and/or materials should be present, they are present in olfactory acceptable amounts, as that term is defined herein.

In an embodiment of the disclosure a compound of formula (I), a compound of formula (Ia) (such as a compound of formula (V)), or a mixture comprising a compound of formula (I) and a compound of formula (Ia) (such as a compound of formula (V)) product in olfactively pure form contains less than 5% by weight of any of the compounds (II), (IIa), (III), (IIIa), (IV), (IVa), (V), (Va), (VI) and/or (VIa) and/or any other material found in the reaction mixture.

In more particular embodiments, a compound of formula (I), a compound of formula (Ia) (such as a compound of formula (V)), or a mixture comprising a compound of formula (I) and a compound of formula (Ia) (such as a compound of formula (V)) product in olfactively pure form contains less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05% by weight of each of the compounds (II), (IIa), (III), (IIIa), (IV), (IVa), (V), (Va), (VI) and/or (VIa) and/or any other material found in the reaction mixture.

In more particular embodiments, a compound of formula (I), a compound of formula (Ia) (such as a compound of formula (V)), or a mixture comprising a compound of formula (I) and a compound of formula (Ia) (such as a compound of formula (V)) product in olfactively pure form contains less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05% by weight of each of the compounds (II), (IIa), (III), (IIIa), (IV), (IVa), (VI) and/or (VIa) and/or any other material found in the reaction mixture.

Non-limiting examples of water miscible and non-water miscible organic solvents suitable for use in the extraction and/or selective crystallization of a compound of formula (I) and/or of a compound of formula (Ia) (such as a compound of formula (V)) include aliphatic hydrocarbons, preferably those having 5 to 8 carbon atoms, such as pentane, cyclopentane, cyclohexane, heptane, octane or cyclooctane, aromatic hydrocarbons, such as toluene, the xylenes, chlorobenzene or dichlorobenzene, aliphatic acyclic and cyclic ethers or alcohols, preferably those having 4 to 8 carbon atoms, such as ethanol, isopropanol, diethyl ether, methyl tert-butyl ether, ethyl tert-butyl ether, dipropyl ether, diisopropyl ether, dibutyl ether, tetrahydrofuran, methyl tetrahydrofuran or esters such as ethyl acetate or n-butyl acetate or ketones such as methyl isobutyl ketone or mixtures thereof. Preferred solvents are heptane, methyl tert-butyl ether (also known as MTBE, tert-butyl methyl ether, tertiary butyl methyl ether, and tBME), diisopropyl ether, tetrahydrofuran, methyl tetrahydrofuran, ethyl acetate and/or mixtures thereof. Preferably, a water miscible solvent such as ethanol is used for the extraction of a compound of formula (I) and/or a compound of formula (Ia) (such as a compound of formula (V)) from the solid phase of the reaction mixture. The use of ethanol may be advantageous because it is easy to handle, it is non-toxic, it is environmentally friendly and it can be produced using renewable raw materials.

The term “% purity” as used herein refers to the percentage of a compound in a material that is the desired compound in the material (for example represented by the percentage ratio of the mass of the desired compound relative to the mass of the entire material). In some embodiments, a compound of formula (I) (e.g., (+)-amberketal) is isolated and purified from an obtained crude product to a purity of at least 80%, 81%, 82%, 83%, 84%, 85%, 86%, 87%, 88%, 89%, 90%, 91%, 92%, 93%, 94%, 95%, 96%, 97%, 98%, or 99%, or 100%.

In some embodiments, a compound of formula (Ia), preferably a compound of formula (V), is isolated and purified from an obtained crude product to a purity of at least 80%, 81%, 82%, 83%, 84%, 85%, 86%, 87%, 88%, 89%, 90%, 91%, 92%, 93%, 94%, 95%, 96%, 97%, 98%, or 99%, or 100%.

In some embodiments, a product comprising a compound of formula (I) (e.g., (+)-amberketal) and a compound of formula (Ia) (such as a compound of formula (V)) is isolated and purified from an obtained crude product to a purity of at least 80%, 81%, 82%, 83%, 84%, 85%, 86%, 87%, 88%, 89%, 90%, 91%, 92%, 93%, 94%, 95%, 96%, 97%, 98%, or 99%, or 100%.

In some embodiments, the concentration of a compound of formula (I) and/or of a compound of formula (Ia) (such as a compound of formula (V)) in a reaction mixture or culture broth obtained by the methods described herein may be from 1 mg/L to 20000 mg/L (20 g/L) or from about 1 mg/L to about 20000 mg/L, or higher such as from 20 g/L to 200 g/L or from about 20 g/L to about 200 g/L, from 100 g/L to 500 g/L or from about 100 g/L to about 500 g/L, from 150 g/L to 500 g/L or from about 150 g/L to about 500 g/L, from 250 g/L to 500 g/L or from about 250 g/L to about 500 g/L, from 300 g/L to 500 g/L or from about 300 g/L to about 500 g/L, from 350 g/L to 500 g/L or from about 350 g/L to about 500 g/L, from 400 g/L to 500 g/L or from about 400 g/L to about 500 g/L, or from 450 g/L to 500 g/L or from about 450 g/L to about 500 g/L. Exemplary concentration values are 1 mg/L or higher, 20 g/L or higher, 50 g/L or higher, 100 g/L or higher, 150 g/L or higher, 200 g/L or higher, 250 g/L or higher, 300 g/L or higher, 350 g/L or higher, 400 g/L or higher, or 450 g/L or higher.

### Compounds of Formulas (III) and (VI)

In some embodiments, a compound of formula (III):

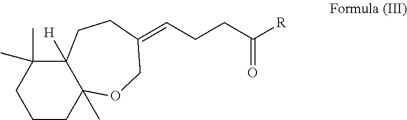

is made as a by-product. In some embodiments, R is selected from H (hydrogen) and a C₁-C₄ alkyl, such as methyl, ethyl, n-propyl, or isopropyl, preferably R is methyl. For example, a compound of formula (III) may have the configuration of formula (IIIa), optionally wherein R is selected from H (hydrogen) and a C₁-C₄ alkyl, such as methyl, ethyl, n-propyl, or isopropyl, preferably wherein R is methyl:

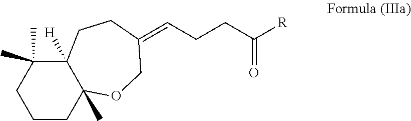

In some embodiments, a compound of formula (VI):

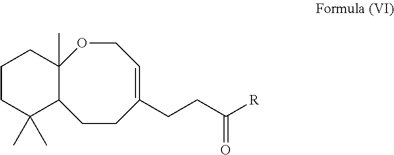

is made as a by-product. In some embodiments, R is selected from H (hydrogen) and a C₁-C₄ alkyl, such as methyl, ethyl, n-propyl, or isopropyl, preferably R is methyl. For example, a compound of formula (VI) may have the configuration of formula (VIa), optionally wherein R is selected from H (hydrogen) and a C₁-C₄ alkyl, such as methyl, ethyl, n-propyl, or isopropyl, preferably wherein R is methyl:

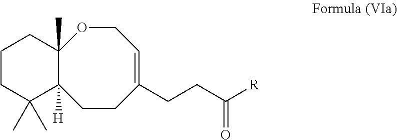

The skilled person understands that the production of specific by-products, such as a compound of formula (III), a compound of formula (IIIa), a compound of formula (VI), and/or a compound of formula (VIa) may depend on the specific substrate used (for example, a compound of formula (II), a compound of formula (IIa), or a mixture comprising a compound of formula (II) and a compound of formula (IIa), as well as the biocatalyst used (as described herein) and/or the bioconversion reaction conditions.

The methods described herein may, for example, make one or more isomers of a compound of formula (III) and/or one or more isomers of a compound of formula (VI). A product, such as a composition, described herein may comprise one or more isomers of a compound of formula (III) and/or one or more isomers of a compound of formula (VI). Accordingly, in some embodiments, a compound of formula (III) having the configuration of formula (IIIa) and/or a compound of formula (VI) having the configuration of formula (VIa), optionally wherein R is selected from H (hydrogen) and a C₁-C₄ alkyl, such as methyl, ethyl, n-propyl, or isopropyl, is made as a by-product. In some embodiments, a product, such as a composition, comprises a compound of formula (III) having the configuration of formula (IIIa). In some embodiments, a product, such as a composition, comprises a compound of formula (VI) having the configuration of formula (VIa). In some embodiments, the only compound of formula (III) made by a method or comprised in a product described herein is a compound having the configuration of formula (IIIa). In some embodiments, the only compound of formula (VI) made by a method or comprised in a product described herein is a compound having the configuration of formula (VIa).

In some embodiments, at least 50 wt % or about 50 wt % of the compounds of formula (III) have the configuration shown in formula (IIIa). In some embodiments, at least 50 wt % or about 50 wt % of the compounds of formula (VI) have the configuration shown in formula (VIa). For example, at least 60 wt % or about 60 wt %, at least 70 wt % or about 70 wt %, at least 80 wt % or about 80 wt %, or at least 90 wt % or about 90 wt % of the compounds of formula (III) may have the configuration shown in formula (IIIa). For example, at least 60 wt % or about 60 wt %, at least 70 wt % or about 70 wt %, at least 80 wt % or about 80 wt %, or at least 90 wt % or about 90 wt % of the compounds of formula (VI) may have the configuration shown in formula (VIa). In some embodiments, compounds having the configuration shown in formula (IIIa) are the only isomers of a compound of formula (III) that are made or comprised in a product, i.e., 100 wt % of the compounds of formula (III) have the configuration shown in formula (IIIa). In some embodiments, compounds having the configuration shown in formula (IIIa) may be equal to or lower than 99 wt % or about 99 wt %, equal to or lower than 95 wt % or about 95 wt %, equal to or lower than 90 wt % or about 90 wt %, equal to or lower than 85 wt % or about 85 wt %, equal to or lower than 80 wt % or about 80 wt %, or equal to or lower than 75 wt % or about 75 wt %, of the compounds of formula (III). In some embodiments, compounds having the configuration shown in formula (VIa) are the only isomers of a compound of formula (VI) that are made or comprised in a product, i.e., 100 wt % of the compounds of formula (VI) have the configuration shown in formula (VIa). In some embodiments, compounds having the configuration shown in formula (VIa) may be equal to or lower than 99 wt % or about 99 wt %, equal to or lower than 95 wt % or about 95 wt %, equal to or lower than 90 wt % or about 90 wt %, equal to or lower than 85 wt % or about 85 wt %, equal to or lower than 80 wt % or about 80 wt %, or equal to or lower than 75 wt % or about 75 wt %, of the compounds of formula (VI).

In some embodiments, from 50 wt % to 100 wt % or from about 50 wt % to about 100 wt %, from 60 wt % to 99 wt % or from about 60 wt % to about 99 wt %, or from 70 wt % to 95 wt % or from about 70 wt % to about 95 wt % of the compounds of formula (III) have the configuration of formula (IIIa). In some embodiments, from 50 wt % to 100 wt % or from about 50 wt % to about 100 wt %, from 60 wt % to 99 wt % or from about 60 wt % to about 99 wt %, or from 70 wt % to 95 wt % or from about 70 wt % to about 95 wt % of the compounds of formula (VI) have the configuration of formula (VIa).

Determination of ratios, amounts, and concentrations of different isomers of a compound of formula (III) and/or of different isomers of a compound of formula (VI) in a mixture may be performed by any method discussed earlier herein.

Suitable reaction conditions for the methods described herein are discussed later herein, and Examples are further given in the experimental section. Additional examples of suitable reaction conditions may be found in WO2021/209482, incorporated herein by reference in its entirety.

### Products Obtained by the Methods Described Herein

In an aspect, there is provided a product, such as a composition, made by the methods described herein. As used herein, “a product made” may be also referred to as “produced”, “obtained by”, or “obtainable by” the methods described herein.

In some embodiments, a composition comprises, consists essentially of, or consists of a compound of formula (I) and a compound of formula (IV). In some embodiments, a composition comprises, consists essentially of, or consists of a compound of formula (I) and a compound of formula (III). The composition may comprise one or more isomers of formula (III), for example a compound having the configuration of formula (IIIa). The composition may further comprise one or more isomers of formula (I), for example a compound of formula (IV). The composition may further comprise one or more isomers of a compound of formula (II), for example an unconverted or unreacted amount of a isomer of a compound of fomula (II).

In some embodiments, a composition comprises, consists essentially of, or consists of a compound of formula (I), a compound of formula (IV), and a compound of formula (III). In some embodiments, a composition comprises, consists essentially of, or consists of a compound of formula (I), a compound of formula (IV), and a compound of formula (IIIa). In some embodiments, a composition comprises, consists essentially of, or consists of a compound of formula (I) and a compound of formula (IIIa).

In some embodiments, a composition comprises, consists essentially of, or consists of a compound of formula (I) and one or more isomers of a compound of formula (I), for example a compound of formula (IV). The composition may, for example, further comprise a compound of formula (III), for example a compound of formula (IIIa). The composition may further comprise one or more isomers of a compound of formula (II), for example an unconverted or unreacted amount of a isomer of a compound of fomula (II).

In some embodiments, a composition comprises, consists essentially of, or consists of a compound of formula (Ia), preferably a compound of formula (V). In some embodiments, a composition comprises, consists essentially of, or consists of a compound of formula (Ia), preferably a compound of formula (V), and a compound of formula (VI). The composition may comprise one or more isomers of formula (VI), for example a compound having the configuration of formula (VIa). The composition may further comprise one or more isomers of formula (Ia). The compositions may further comprise one or more isomers of a compound of formula (IIa), for example an unconverted or unreacted amount of a isomer of a compound of formula (IIa).

In some embodiments, a composition comprises, consists essentially of, or consists of a compound of formula (I) and a compound of formula (Ia). In some embodiments, a composition comprises, consists essentially of, or consists of a compound of formula (I) and a compound of formula (V). The composition may further comprise a compound of formula (IV). The composition may further comprise an isomer of a compound of formula (Ia). The composition may further comprise a compound of formula (III), for example a compound of formula (IIIa). The composition may further comprise a compound of formula (VI), for example a compound of formula (VIa). The composition may further comprise one or more isomers of a compound of formula (II), for example an unconverted or unreacted amount of a isomer of a compound of formula (II). The composition may further comprise one or more isomers of a compound of formula (IIa), for example an unconverted or unreacted amount of a isomer of a compound of fomula (IIa). In some embodiments, the composition does not comprise a compound of formula (III). In some embodiments, the composition does not comprise a compound of formula (IIIa). In some embodiments, the composition does not comprise a compound of formula (VI). In some embodiments, the composition does not comprise a compound of formula (VIa).

In some embodiments, in compounds of formula (I) and its isomers, for example compounds of formula (IV), compounds of formula (Ia) and its isomers, for example compound of formula (V), compounds of formula (II) and its isomers, compounds of formula (IIa) and its isomers, compounds of formula (III) and its isomers, for example compounds of formula (IIIa), and compounds of formula (VI) and its isomers, for example compounds of formula (VIa), present in the compositions described herein, R is selected from H (hydrogen) and a C₁-C₄ alkyl, such as methyl, ethyl, n-propyl, or isopropyl, preferably R is methyl.

In some embodiments, the ratio of a compound of formula (I) to a compound of formula (III) (e.g., a compound of formula (IIIa)) in the compositions described herein may be from 60:40 to 99:1 or from about 60:40 to about 99:1. In some embodiments, the ratio of a compound of formula (I) to a compound of formula (III) in the compositions described herein may be from 65:35 to 99:1 or from about 65:35 to about 99:1, from 70:30 to 99:1 or from about 70:30 to about 99:1, from 75:25 to 99:1 or from about 75:25 to about 99:1, from 80:20 to 99:1 or from about 80:20 to about 99:1, from 85:15 to 99:1 or from about 85:15 to about 99:1, from 90:10 to 99:1 or from about 90:10 to about 99:1, from 95:5 to 99:1 or from about 95:5 to about 99:1, from 65:35 to 98:2 or from about 65:35 to about 98:2, from 70:30 to 97:3 or from about 70:30 to about 97:3, from 75:25 to 96:4 or from about 75:25 to about 96:4, from 80:20 to 95:5 or from about 80:20 to about 95:5, from 85:15 to 90:10 or from about 85:15 to about 90:10.

In some embodiments, the ratio of a compound of formula (I) to a compound of formula (II) in the compositions, such as a crude product, described herein may be from 90:10 to 100:0 or from about 90:10 to about 100:0. In some embodiments, the ratio of a compound of formula (I) to a compound of formula (II) in the compositions, such as a crude product, described herein may be from 92:8 to 100:0 or from about 92:8 to about 100:0, from 94:6 to 100:0 or from about 94:6 to about 100:0, from 95:5 to 100:0 or from about 95:5 to about 100:0, from 96:4 to 99.5:0.5 or from about 96:4 to about 99.5:0.5, from 97:3 to 99:1 or from about 97:3 to about 99:1, from 98:2 to 99:1 or from about 98:2 to about 99:1.

In some embodiments, the ratio of a compound of formula (Ia), preferably of a compound of formula (V), to a compound of formula (VI) (e.g., a compound of formula (VIa)) in the compositions described herein may be from 60:40 to 99:1 or from about 60:40 to about 99:1. In some embodiments, the ratio of a compound of formula (Ia), preferably of a compound of formula (V), to a compound of formula (VI) in the compositions described herein may be from 65:35 to 99:1 or from about 65:35 to about 99:1, from 70:30 to 99:1 or from about 70:30 to about 99:1, from 75:25 to 99:1 or from about 75:25 to about 99:1, from 80:20 to 99:1 or from about 80:20 to about 99:1, from 85:15 to 99:1 or from about 85:15 to about 99:1, from 90:10 to 99:1 or from about 90:10 to about 99:1, from 95:5 to 99:1 or from about 95:5 to about 99:1, from 65:35 to 98:2 or from about 65:35 to about 98:2, from 70:30 to 97:3 or from about 70:30 to about 97:3, from 75:25 to 96:4 or from about 75:25 to about 96:4, from 80:20 to 95:5 or from about 80:20 to about 95:5, from 85:15 to 90:10 or from about 85:15 to about 90:10.

In some embodiments, the ratio of a compound of formula (Ia), preferably of a compound of formula (V), to a compound of formula (IIa) in the compositions, such as a crude product, described herein may be from 90:10 to 100:0 or from about 90:10 to about 100:0. In some embodiments, the ratio of a compound of formula (Ia), preferably of a compound of formula (V), to a compound of formula (IIa) in the compositions, such as a crude product, described herein may be from 92:8 to 100:0 or from about 92:8 to about 100:0, from 94:6 to 100:0 or from about 94:6 to about 100:0, from 95:5 to 100:0 or from about 95:5 to about 100:0, from 96:4 to 99.5:0.5 or from about 96:4 to about 99.5:0.5, from 97:3 to 99:1 or from about 97:3 to about 99:1, from 98:2 to 99:1 or from about 98:2 to about 99:1.

Determination of ratios, amounts, and concentrations of a compound of formula (I) and its isomers, for example a compound of formula (IV), a compound of formula (Ia) and its isomers, for example a compound of formula (V), a compound of formula (II) and its isomers, a compound of formula (IIa) and its isomers, a compound of formula (III) and its isomers, for example a compound of formula (IIIa), and a compound of formula (VI) and its isomers, for example a compound of formula (VI), in a composition may be performed by any method discussed earlier herein.

In some embodiments, a composition obtained by or obtainable by the methods described herein comprises a compound of formula (I) and a compound of formula (Ia) (such as a compound of formula (V)) in a solid form, preferably in an amorphous or crystalline form.

### Fragrance Compositions

Products, such as compositions, made by the methods described herein may be comprised in a fragrance composition. Accordingly, there is further provided the use of a composition as described herein for the manufacture of a fragrance composition. In some embodiments, a fragrance composition comprises a compound of formula (I). Optionally, a fragrance composition comprises a isomer of a compound of formula (I), for example a compound of formula (IV). In some embodiments, a fragrance composition comprises a compound of formula (Ia), preferably a compound of formula (V). In some embodiments, a fragrance composition comprises a compound of formula (I) and a compound of formula (Ia). In some embodiments, a composition comprises a compound of formula (I) and a compound of formula (V). Optionally, a fragrance composition comprises an isomer of a compound of formula (Ia). A “fragrance composition” as used herein includes any composition that comprises a compound of formula (I), and optionally one or more isomers of a compound of formula (I) such as for example a compound of formula (IV), and a base material. It further includes any composition that comprises a compound of formula (Ia), and a base material. It further includes any composition that comprises a compound of formula (V), and optionally one or more other isomers of a compound of formula (Ia), and a base material. It further includes any composition that comprises a compound of formula (I), a compound of formula (Ia), and a base material. It further includes any composition that comprises a compound of formula (I), a compound of formula (V), and a base material, optionally additional comprising one or more isomers of a compound of formula (I) and/or one ore more other isomers of a compound of formula (Ia).

As used herein, a “base material” may be understood to include all known fragrance ingredients selected from the extensive range of natural products and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, diluents, and other auxiliary agents commonly used in the art; examples of which can be found in standard handbooks such as Perfume Engineering: Design, Performance and Classification (2012), Miguel Teixeira et al., Butterworth-Heinemann, UK, incorporated herein by reference in its entirety.

Suitable fragrance ingredients are further commercially available. Non-limiting examples of such ingredients include:
essential oils and extracts, e.g., castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g., cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl) pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl) pentan-2-ol); terpineol (2-(4-methylcyclohex-3-en-1-yl) propan-2-ol); or Timberol™ (1-(2,2,6-trimethylcyclohexyl) hexan-3-ol); 2,4,7-trimethylocta-2,6-dien-1-ol, and/or [1-methyl-2 (5-methylhex-4-en-2-yl)cyclopropyl]-methanol;
aldehydes and ketones, e.g., anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl) ethanone); hydroxycitronellal (7-hydroxy-3,7-dimethyloctanal); Iso E Super® (1-(2,3,8,8-tetramethyl-1,2,3,4,5,6,7,8-octahydronaphthalen-2-yl) ethanone); Isoraldeine® ((E)-3-methyl-4-(2,6,6-trimethylcyclohex-2-en-1-yl) but-3-en-2-one); 3-(4-isobutyl-2-methylphenyl) propanal; maltol; methyl cedryl ketone; methylionone; verbenone; and/or vanillin;
ether and acetals, e.g., Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicycle[4.1.0]heptane-2,5′-[1,3]dioxane]);
macrocycles, e.g., ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g., isobutylquinoline (2-isobutylquinoline).

As used herein, a “carrier material” may be understood to be a material which is practically neutral from an odorant point of view, i.e., a material that does not significantly alter the organoleptic properties of odorants. The term “diluent” may be understood to include any diluent conventionally used in conjunction with odorants, examples being diethyl phthalate (DEP), dipropylene glycol (DPG), isopropyl myristate (IPM), triethyl citrate (TEC) and alcohol (e.g., ethanol). The term “auxiliary agent” may be understood to include any ingredient that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same, such as an anti-oxidant adjuvant. An anti-oxidant may be selected, for example, from Tinogard® TT (BASF), Tinogard® Q (BASF), tocopherol (including its isomers, CAS 59-02-9; 364-49-8; 18920-62-2; 121854-78-2), 2,6-bis (1,1-dimethylethyl)-4-methylphenol (BHT, CAS 128-37-0) and related phenols, hydroquinones (CAS 121-31-9). An auxiliary agent may also be an ingredient that provides additional benefits such as imparting colour or texture to a fragrance composition. An auxiliary agent may also be an ingredient that imparts resistance to light or an increase in chemical stability to one or more ingredients contained in a fragrance composition. Fragrance ingredients, carrier materials, diluents, and auxiliary agents discussed herein are to be understood as non-limiting examples; the skilled person is aware of suitable base materials commonly used in the art, further examples of which being available in standard handbooks such as Perfume Engineering: Design, Performance and Classification (supra).

A compound of formula (I), a compound of formula (Ia) (such as a compound of formula (V)), and a mixture comprising a compound of formula (I) and a compound of formula (Ia) (such as a compound of formula (V)), as described herein, may be further comprised in multiple compositions including, but not limited to, a fine fragrance or a consumer product such as fabric care, toiletries, beauty care and cleaning products, detergent products, and soap products, including essentially all products where the currently available (+)-amberketal ingredients are used commercially.

The disclosure further provides a consumer product comprising a composition or a fragrance composition as described herein, including any embodiment thereof. The consumer product may, for example, be a cosmetic product (e.g., an eau de parfum or eau de toilette), a cleaning product, a detergent product, or a soap product.

Fragrances and consumer products comprising a mixture comprising a compound of formula (I) and a compound of formula (Ia) (such as a compound of formula (V)) may be advantageous, as they exhibit unique olfactory properties.

Accordingly, in some embodiments, a fragrance composition or a consumer product comprises a composition comprising a compound of formula (I) and a compound of formula (Ia) (such as a compound of formula (V), wherein said composition is obtained by or is obtainable by the methods described herein. In some embodiments, the compound of formula (I) and the compound of formula (Ia) (such as a compound of formula (V)) is in a solid form, preferably in an amorphous or crystalline form.

### Starting Materials and Intermediates

In an aspect, the disclosure provides the starting materials and intermediates used in the methods described herein.

Also provided herein is a mixture comprising, consisting essentially of, or consisting of a compound of formula (II). For example, a mixture may comprise, consist essentially of, or consist of a compound of formula (II) which is such that the double bond between C-8 and C-9 is in E-configuration and the double bond between C-4 and C-5 is in E-configuration (E,E-isomer) and a compound of formula (II) which is such that the double bond between C-8 and C-9 is in E-configuration and the double bond between C-4 and C-5 is in Z-configuration (E,Z-isomer). In some embodiments, the mixture comprises three of the isomers of a compound of formula (II), for example the E,Z-isomer, the E,E-isomer, and one of the Z,E-isomer or the Z,Z-isomer. In some embodiments, the mixture comprises all four isomers of a compound of formula (II), i.e., the E,Z-isomer, the E,E-isomer, the Z,E-isomer, and the Z,Z-isomer.

In some embodiments, R is selected from H (hydrogen) and a C₁-C₄ alkyl, such as methyl, ethyl, n-propyl, or isopropyl, preferably R is methyl.

Also provided herein is a mixture comprising, consisting essentially of, or consisting of a compound of formula (IIa). For example, a mixture may comprise, consist essentially of, or consist of a compound of formula (IIa) which is such that the double bond between C-6 and C-7 is in E-configuration and the double bond between C-2 and C-3 is in E-configuration (E,E-isomer) and a compound of formula (IIa) which is such that the double bond between C-6 and C-7 is in E-configuration and the double bond between C-2 and C-3 is in Z-configuration (E,Z-isomer). In some embodiments, the mixture comprises three of the isomers of a compound of formula (IIa), for example the E,Z-isomer, the E,E-isomer, and one of the Z,E-isomer or the Z,Z-isomer. In some embodiments, the mixture comprises four isomers of a compound of formula (IIa), i.e., the E,Z-isomer, the E,E-isomer, the Z,E-isomer, and the Z,Z-isomer.

In some embodiments, R is selected from H (hydrogen) and a C₁-C₄ alkyl, such as methyl, ethyl, n-propyl, or isopropyl, preferably R is methyl.

Also provided herein is a mixture comprising, consisting essentially of, or consisting of a compound of formula (II) and a compound of formula (IIa). For example, a mixture may comprise, consist essentially of, or consist of a compound of formula (II) which is such that the double bond between C-8 and C-9 is in E-configuration and the double bond between C-4 and C-5 is in E-configuration (E,E-isomer) and a compound of formula (IIa) which is such that the double bond between C-6 and C-7 is in E-configuration and the double bond between C-2 and C-3 is in E-configuration (E,E-isomer). For example, a mixture may comprise, consist essentially of, or consist of a compound of formula (II) which is such that the double bond between C-8 and C-9 is in E-configuration and the double bond between C-4 and C-5 is in E-configuration (E,E-isomer) and a compound of formula (IIa) which is such that the double bond between C-6 and C-7 is in E-configuration and the double bond between C-2 and C-3 is in Z-configuration (E,Z-isomer). For example, a mixture may comprise, consist essentially of, or consist of a compound of formula (II) which is such that the double bond between C-8 and C-9 is in E-configuration and the double bond between C-4 and C-5 is in Z-configuration (E,Z-isomer) and a compound of formula (IIa) which is such that the double bond between C-6 and C-7 is in E-configuration and the double bond between C-2 and C-3 is in E-configuration (E,E-isomer). For example, a mixture may comprise, consist essentially of, or consist of a compound of formula (II) which is such that the double bond between C-8 and C-9 is in E-configuration and the double bond between C-4 and C-5 is in Z-configuration (E,Z-isomer) and a compound of formula (IIa) which is such that the double bond between C-6 and C-7 is in E-configuration and the double bond between C-2 and C-3 is in Z-configuration (E,Z-isomer).

For example, a mixture may comprise, consist essentially of, or consist of a compound of formula (II) which is such that the double bond between C-8 and C-9 is in E-configuration and the double bond between C-4 and C-5 is in E-configuration (E,E-isomer), a compound of formula (II) which is such that the double bond between C-8 and C-9 is in E-configuration and the double bond between C-4 and C-5 is in Z-configuration (E,Z-isomer), a compound of formula (IIa) which is such that the double bond between C-6 and C-7 is in E-configuration and the double bond between C-2 and C-3 is in E-configuration (E,E-isomer), and a compound of formula (IIa) which is such that the double bond between C-6 and C-7 is in E-configuration and the double bond between C-2 and C-3 is in Z-configuration (E,Z-isomer). Optionally, the mixture may further comprise one or more other isomers of a compound of formula (II) and/or of a compound of formula (IIa).

In some embodiments, R is selected from H (hydrogen) and a C₁-C₄ alkyl, such as methyl, ethyl, n-propyl, or isopropyl, preferably R is methyl.

In a mixture comprising an E,Z-isomer and an E,E-isomer of a compound of formula (II), the ratio of the E,Z-isomer to the E,E-isomer may be equal to or greater than 10:90 or about 10:90. In some embodiments, the ratio is equal to or greater than 20:80 or about 20:80, equal to or greater than 30:70 or about 30:70, equal to or greater than 40:60 or about 40:60, equal to or greater than 50:50 or about 50:50, equal to or greater than 60:40 or about 60:40, equal to or greater than 70:30 or about 70:30, equal to or greater than 80:20 or about 80:20, equal to or greater than 85:15 or about 85:15, equal to or greater than 90:10 or about 90:10, equal to or greater than 95:5 or about 95:5, or equal to or greater than 99:1 or about 99:1.

In a mixture comprising an E,Z-isomer and an E,E-isomer of a compound of formula (II), the ratio of the E,Z-isomer to the E,E-isomer may be equal to or lower than 99:1 or about 99:1. In some embodiments, the ratio is equal to or lower than 95:5 or about 95:5, equal to or lower than 90:10 or about 90:10, equal to or lower than 85:15 or about 85:15, equal to or lower than 80:20 or about 80:20, equal to or lower than 70:30 or about 70:30, equal to or lower than 60:40 or about 60:40, equal to or lower than 50:50 or about 50:50, equal to or lower than 40:60 or about 40:60, equal to or lower than 30:70 or about 30:70, equal to or lower than 20:80 or about 20:80, or equal to or lower than 10:90 or about 10:90.

In a mixture comprising an E,Z-isomer and an E, E-isomer of a compound of formula (II), the ratio of the E,Z-isomer to the E, E-isomer may be from 10:90 to 99:1 or from about 10:90 to about 99:1, from 10:90 to 90:10 or from about 10:90 to about 90:10 or from about 5:95 to about 95:5 or from about 4:96 to about 96:4 or from about 3:97 to about 97:3 or from about 2:98 to about 98:2 or from about 1:99 to about 99:1 or or from about 20:80 to about 80:20, from 50:50 to 80:20 or from about 50:50 to about 80:20, or from 60:40 to 80:20 or from about 60:40 to about 80:20. Optionally, the mixture may further comprise one or more other isomers of a compound of formula (II) and/or of a compound of formula (IIa).

In a mixture comprising an E,Z-isomer and an E,E-isomer of a compound of formula (IIa), the ratio of the E,Z-isomer to the E,E-isomer may be equal to or greater than 10:90 or about 10:90. In some embodiments, the ratio is equal to or greater than 20:80 or about 20:80, equal to or greater than 30:70 or about 30:70, equal to or greater than 40:60 or about 40:60, equal to or greater than 50:50 or about 50:50, equal to or greater than 60:40 or about 60:40, equal to or greater than 70:30 or about 70:30, equal to or greater than 80:20 or about 80:20, equal to or greater than 85:15 or about 85:15, equal to or greater than 90:10 or about 90:10, equal to or greater than 95:5 or about 95:5, or equal to or greater than 99:1 or about 99:1.

In a mixture comprising an E,Z-isomer and an E,E-isomer of a compound of formula (IIa), the ratio of the E,Z-isomer to the E,E-isomer may be equal to or lower than 99:1 or about 99:1. In some embodiments, the ratio is equal to or lower than 95:5 or about 95:5, equal to or lower than 90:10 or about 90:10, equal to or lower than 85:15 or about 85:15, equal to or lower than 80:20 or about 80:20, equal to or lower than 70:30 or about 70:30, equal to or lower than 60:40 or about 60:40, equal to or lower than 50:50 or about 50:50, equal to or lower than 40:60 or about 40:60, equal to or lower than 30:70 or about 30:70, equal to or lower than 20:80 or about 20:80, or equal to or lower than 10:90 or about 10:90.

In a mixture comprising an E,Z-isomer and an E,E-isomer of a compound of formula (IIa), the ratio of the E,Z-isomer to the E,E-isomer may be from 10:90 to 99:1 or from about 10:90 to about 99:1, from 10:90 to 90:1 or from about 10:90 to about 90:1, from 20:80 to 80:20 or from about 20:80 to about 80:20, from 50:50 to 80:20 or from about 50:50 to about 80:20, or from 60:40 to 80:20 or from about 60:40 to about 80:20. Optionally, the mixture may further comprise one or more other isomers of a compound of formula (II) and/or of a compound of formula (IIa).

In a mixture comprising a compound of formula (II) and a compound of formula (IIa), the ratio of the compound of formula (II) to the compound of formula (IIa) may be equal to or greater than 50:50 or about 50:50, equal to or greater than 60:40 or about 60:40, equal to or greater than 70:30 or about 70:30, equal to or greater than 80:20 or about 80:20, equal to or greater than 85:15 or about 85:15, equal to or greater than 90:10 or about 90:10, equal to or greater than 95:5 or about 95:5, or equal to or greater than 99:1 or about 99:1.

In a mixture comprising a compound of formula (II) and a compound of formula (IIa), the ratio of the compound of formula (II) to the compound of formula (IIa) may be equal to or lower than 99:1 or about 99:1. In some embodiments, the ratio is equal to or lower than 95:5 or about 95:5, equal to or lower than 90:10 or about 90:10, equal to or lower than 85:15 or about 85:15, equal to or lower than 80:20 or about 80:20, equal to or lower than 70:30 or about 70:30, equal to or lower than 60:40 or about 60:40, equal to or lower than 50:50 or about 50:50, equal to or lower than 40:60 or about 40:60, equal to or lower than 30:70 or about 30:70, equal to or lower than 20:80 or about 20:80, or equal to or lower than 10:90 or about 10:90.

In a mixture comprising a compound of formula (II) and a compound of formula (IIa), the ratio of the compound of formula (II) to the compound of formula (IIa) may be from 10:90 to 99:1 or from about 10:90 to about 99:1, from 10:90 to 90:1 or from about 10:90 to about 90:1, from 20:80 to 80:20 or from about 20:80 to about 80:20, from 50:50 to 80:20 or from about 50:50 to about 80:20, or from 60:40 to 80:20 or from about 60:40 to about 80:20.

### Squalene-Hopene Cyclase (SHC) Enzyme

The methods described herein utilize a squalene-hopene cyclase (SHC) enzyme as described herein.

In some embodiments, a squalene-hope cyclase enzyme described herein may comprise an amino acid sequence having at least 30%, 40%, 50%, 60%, or 70%, preferably at least 70%, identity or similarity with the sequence of SEQ ID NO: 1 or SEQ ID NOs: 43-49, preferably with the sequence of SEQ ID NO: 1. SEQ ID NO: 1 represents an SHC enzyme derived from Bacillus megaterium (BmeSHC). SEQ ID NO: 43 represents an SHC enzyme derived from Alicyclobacillus acidocaldarius (AacSHC). SEQ ID NOs: 44 and 45 represent SHC enzymes derived from Zymomonas mobilis (ZmoSHC1 and ZmoSHC2, respectively). SEQ ID NO: 46 represents an SHC enzyme derived from Bradyrhizobium japonicum (BjaSHC). SEQ ID NO: 47 represents an SHC enzyme derived from Thermosynechococcus elongatus (TelSHC). SEQ ID NO: 48 represents an SHC enzyme derived from Acetobacter pasteurianus (ApaSHC). SEQ ID NO: 49 represents an SHC enzyme derived from Gluconobacter morbifer (GmoSHC). A further description of these enzymes may be found in WO2021/209482.

In some embodiments, a squalene-hopene cyclase (SHC) enzyme described herein comprises an amino acid sequence having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, or 100% identity or similarity with the sequence of SEQ ID NO: 1 or SEQ ID NOs: 43-49, preferably with the sequence of SEQ ID NO: 1. In some embodiments, the identity or similarity is at least 30%. In some embodiments, the identity or similarity is at least 35%. In some embodiments, the identity or similarity is at least 40%. In some embodiments, the identity or similarity is at least 45%. In some embodiments, the identity or similarity is at least 50%. In some embodiments, the identity or similarity is at least 55%. In some embodiments, the identity or similarity is at least 60%. In some embodiments, the identity or similarity is at least 65%. In some embodiments, the identity or similarity is at least 70%. In some embodiments, the identity or similarity is at least 75%. In some embodiments, the identity or similarity is at least 80%. In some embodiments, the identity or similarity is at least 85%. In some embodiments, the identity or similarity is at least 90%. In some embodiments, the identity or similarity is at least 95%. In some embodiments, the identity or similarity is at least 95.5%. In some embodiments, the identity or similarity is at least 96%. In some embodiments, the identity or similarity is at least 96.5%. In some embodiments, the identity or similarity is at least 97%. In some embodiments, the identity or similarity is at least 97.5%. In some embodiments, the identity or similarity is at least 98%. In some embodiments, the identity or similarity is at least 98.5%. In some embodiments, the identity or similarity is at least 99%. In some embodiments, the identity or similarity is at least 99.5%. In some embodiments, the identity or similarity is less than 100%, i.e. the amino acid sequence is not identical to SEQ ID NO: 1 or SEQ ID NO: 43-49, preferably to SEQ ID NO: 1. Definitions of sequence “identity” and “similarity”, as well as methods for their determination, are provided in the section entitled “general definitions” later herein.

SHC enzymes described herein may be derived from an SHC enzyme represented by SEQ ID NO: 1 or SEQ ID NOs: 43-49, preferably from an SHC enzyme represented by SEQ ID NO: 1, by introduction of a modification to its sequence. Such enzymes may also be referred to herein as “SHC variants”, “SHC mutants”, or “SHC derivatives”. SHC enzymes described herein may also be derived from other SHC variants by introduction of an additional modification to the sequence of an existing SHC variant. The SHC enzymes described herein may be not naturally occurring.

In other words, the term “variant”, such as an SHC variant, is to be understood as a polypeptide (enzyme) described herein which comprises one or more sequence modifications in comparison to the polypeptide from which it is derived. The polypeptide from which a variant is derived may also be referred to herein as the parent or reference polypeptide (i.e., parent or reference SHC enzyme). A parent SHC enzyme may be a wild-type enzyme. A parent SHC enzyme may be a homolog, ortholog, or paralog of a wild-type polypeptide. A parent SHC enzyme may be another variant, i.e., an enzyme that is derived from introduction of additional modifications in its amino acid sequence as compared to a previously obtained variant enzyme. Thus, SHC enzymes described herein may be derived from an “earlier generation” of SHC variants, and may exhibit improved properties compared to their parent SHC enzymes. Examples of sequence modifications that may be comprised in a variant enzyme are amino acid substitutions, deletions, insertions, N-terminal truncations, C-terminal truncations, or combinations thereof. Variant enzymes may, for example, be synthetically made or made by cellular (or in vitro) production, after modifying the nucleotide sequence encoding for said enzymes using mutagenesis techniques known to the skilled person, such as, random mutagenesis, site-directed mutagenesis, directed evolution, gene shuffling, CRISPR/Cas-mediated mutagenesis and the like, examples of which also being available in standard handbooks such as In Vitro Mutagenesis: Methods and Protocols (Methods in Molecular Biology 1498), 1^{st} Edition, Reeves A. (Ed), Humana Press (2017), incorporated herein by reference in its entirety. In some embodiments, an SHC enzyme described herein is synthetically made. In some embodiments, an SHC enzyme described herein is produced by a recombinant host cell.

A sequence modification of an SHC described herein as compared to its parent SHC enzyme, such as an SHC enzyme represented by SEQ ID NO: 1 or SEQ ID NOs: 43-49, preferably by SEQ ID NO: 1, may be identified via direct comparison of their respective amino acid sequences or of the nucleotide sequences of the nucleic acids encoding said enzymes, using standard bioinformatics algorithms available in the art and further discussed in the section entitled “general definitions” later herein. These algorithms typically utilize routine sequence alignment methods, in which specific nucleotides or amino acid residues corresponding to specific positions of a sequence are matched to the corresponding positions of a reference sequence it is being aligned against.

Taking SEQ ID NO: 1 as an example, and using such methods, the skilled person can e.g., easily identify which amino acid positions in an SHC enzyme correspond to, for example, positions 2, 5, 35, 116, 166, 211, 212, 317, 355, 382, 399, 483, 539, and 585 in SEQ ID NO: 1 (or any other position in SEQ ID NO: 1), if SEQ ID NO: 1 is used as a reference sequence and the SHC enzyme amino acid sequence in question is aligned against it. Similarly, the positions of the corresponding nucleotides encoding specific amino acid residues may be identified, if the nucleotide sequence of the nucleic acids encoding SEQ ID NO: 1 and the SHC enzyme in question are aligned instead. In this regard, the skilled person understands that the methionine (M) residue at the N-terminus end of SEQ ID NO: 1 corresponds to position 1, that the serine(S) residue at the C-terminus end of SEQ ID NO: 1 corresponds to position 625, and that the amino acids in between the N- and C-terminus ends of SEQ ID NO: 1 correspond to positions 2-624, respectively.

An amino acid substitution refers to a sequence modification that replaces an amino acid residue in a parent (reference) amino acid sequence (or a nucleotide in a nucleotide sequence of a nucleic acid encoding the amino acid sequence) which results in a variant (derivative) sequence that has the same number of amino acids. An amino acid substitution may correspond to a substitution by any other amino acid. An amino acid substitution may be conservative. A definition of “conservative” substitutions is provided later herein. An amino acid substitution may correspond to multiple specific amino acid positions of a parent SHC enzyme sequence, such as a sequence represented by SEQ ID NO: 1 or SEQ ID NO: 43-49, preferably by SEQ ID NO: 1. In embodiments wherein multiple amino acids are substituted, they may correspond to consecutive positions, to positions that are not consecutive, or to positions that are spatially apart in the polypeptide sequence.

In some embodiments, an SHC enzyme described herein comprises one or more amino acid substitutions relative to SEQ ID NO: 1. Preferred positions for substitutions may be selected from the group of positions 2, 5, 35, 116, 166, 211, 212, 317, 355, 382, 399, 483, 539, and 585 in SEQ ID NO: 1. In some embodiments, a preferred SHC enzyme described herein comprises one or more amino acid substitutions relative to SEQ ID NO: 1 at one or more positions corresponding to positions 2, 5, 35, 166, 211, 212, 355, 483, and 539 in SEQ ID NO: 1. Preferably, the one or more amino acid substitutions relative to SEQ ID NO: 1 are at one or more positions corresponding to position 2, 5, 35, 166, 211, 212, 483, and 539 in SEQ ID NO: 1. More preferably, the one or more amino acid substitutions relative to SEQ ID NO: 1 are at one or more positions corresponding to position 2, 5, 35, 166, 211, 483, and 539 in SEQ ID NO: 1.

In some embodiments, an SHC enzyme described herein comprises at least one, at least two, at least three, at least four, at least five, at least six, at least seven, at least eight, at least nine, at least ten, at least eleven, at least twelve, at least thirteen, or at least fourteen amino acid substitutions relative to SEQ ID NO: 1. In some embodiments, at least one amino acid has been substituted relative to SEQ ID NO: 1. In some embodiments, at least two amino acids have been substituted relative to SEQ ID NO: 1. In some embodiments, at least three amino acids have been substituted relative to SEQ ID NO: 1. In some embodiments, at least four amino acids have been substituted relative to SEQ ID NO: 1. In some embodiments, at least five amino acids have been substituted relative to SEQ ID NO: 1. In some embodiments, at least six amino acids have been substituted relative to SEQ ID NO: 1. In some embodiments, at least seven amino acids have been substituted relative to SEQ ID NO: 1. In some embodiments, at least eight amino acids have been substituted relative to SEQ ID NO: 1. In some embodiments, at least nine amino acids have been substituted relative to SEQ ID NO: 1. In some embodiments, at least ten amino acids have been substituted relative to SEQ ID NO: 1. In some embodiments, at least eleven amino acids have been substituted relative to SEQ ID NO: 1. In some embodiments, at least twelve amino acids have been substituted relative to SEQ ID NO: 1. In some embodiments, at least thirteen amino acids have been substituted relative to SEQ ID NO: 1. In some embodiments, at least fourteen amino acids have been substituted relative to SEQ ID NO: 1. Preferred positions for substitutions may be selected from the group of positions 2, 5, 35, 116, 166, 211, 212, 317, 355, 382, 399, 483, 539, and 585, preferably 2, 5, 35, 166, 211, 212, 355, 483, and 539, more preferably 2, 5, 35, 166, 211, 212, 483 and 539, most preferably 2, 5, 35, 166, 211, 483, and 539.

In some embodiments, an SHC enzyme described herein comprises one to seven, preferably two to six, more preferably three to five amino acid substitutions relative to SEQ ID NO: 1. In some embodiments, an SHC enzyme described herein comprises one to seven, preferably two to six, more preferably three to five amino acid substitutions at one or more positions corresponding to positions 2, 5, 35, 116, 166, 211, 212, 317, 355, 382, 399, 483, 539, and 585, preferably 2, 5, 35, 166, 211, 212, 355, 483, and 539, more preferably 2, 5, 35, 166, 211, 212, 483, and 539 in SEQ ID NO: 1, most preferably 2, 5, 35, 166, 211, 483, and 539 in SEQ ID NO: 1.

As used herein, “conservative” amino acid substitutions refer to the interchangeability of residues having similar side chains. Conservative amino acid substitutions may be made, for instance, on the basis of similarity in polarity, charge, size, solubility, hydrophobicity, hydrophilicity, and/or the amphipathic nature of the amino acid residues involved.

Examples of similar classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic | Ser (S), Thr (T), Asn (N), and Gln (Q) |
| Uncharged Residues | |
| Aliphatic | Gly (G), Ala (A), |
| Uncharged Residues | Val (V), Leu (L), and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues:

| | |
|---|---|
| Alcohol group-containing residues | S and T |
| Aliphatic residues | I, L, V, and M |
| Cycloalkenyl-associated residues | F, H, W, and Y |
| Hydrophobic | A, C, F, G, H, I, L, |
| residues | M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved | A, C, D, E, G, H, K, |
| in turn formation | N, Q, R, S, P and T |
| Flexible residues | Q, T, K, S, G, P, D, E, and R |
| Residues that influence | G, P |
| chain orientation | |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gin; IIe to Leu or Val; Leu to IIe or Val; Lys to Arg; Gln or Glu; Met to Leu or IIe; Phe to Met, Leu or Tyr; Ser to Thr; Thr to Ser; Trp to Tyr; Tyr to Trp or Phe; and, Val to IIe or Leu.

Preferred substutions occurring at the preferred substituted positions corresponding to specific positions in SEQ ID NO: 1 described herein are indicated below.

In some embodiments, an SHC enzyme described herein comprises an amino acid sequence in which the isoleucine (I) corresponding to position 2 in SEQ ID NO: 1 has been substituted by any amino acid, preferably by asparagine (N), serine(S), threonine (T), or glutamine (Q), more preferably by asparagine (N).

In some embodiments, an SHC enzyme described herein comprises an amino acid sequence in which the leucine (L) corresponding to position 5 in SEQ ID NO: 1 has been substituted by any amino acid, preferably by proline (P), methionine (M), or cysteine (C), more preferably by proline (P).

In some embodiments, an SHC enzyme described herein comprises an amino acid sequence in which the threonine (T) corresponding to position 35 in SEQ ID NO: 1 has been substituted by any amino acid, preferably by alanine (A), isoleucine (I), valine (V), glycine (G), or leucine (L), more preferably by alanine (A).

In some embodiments, an SHC enzyme described herein comprises an amino acid sequence in which the isoleucine (I) corresponding to position 116 in SEQ ID NO: 1 has been substituted by any amino acid, preferably by threonine (T), asparagine (N), serine(S), or glutamine (Q), more preferably by threonine (T).

In some embodiments, an SHC enzyme described herein comprises an amino acid sequence in which the threonine (T) corresponding to position 166 in SEQ ID NO: 1 has been substituted by any amino acid, preferably by alanine (A), isoleucine (I), valine (V), glycine (G), or leucine (L), more preferably by alanine (A).

In some embodiments, an SHC enzyme described herein comprises an amino acid sequence in which the glutamic acid (E) corresponding to position 211 in SEQ ID NO: 1 has been substituted by any amino acid, preferably by valine (V), alanine (A), isoleucine (I), glycine (G), or leucine (L), more preferably by valine (V).

In some embodiments, an SHC enzyme described herein comprises an amino acid sequence in which the serine(S) corresponding to position 212 in SEQ ID NO: 1 has been substituted by any amino acid, preferably by arginine (R), lysine (K), or histidine (H), more preferably by arginine (R).

In some embodiments, an SHC enzyme described herein comprises an amino acid sequence in which the leucine (L) corresponding to position 317 in SEQ ID NO: 1 has been substituted by any amino acid, preferably by methionine (M), proline (P), or cysteine (C), more preferably by methionine (M).

In some embodiments, an SHC enzyme described herein comprises an amino acid sequence in which the alanine (A) corresponding to position 355 in SEQ ID NO: 1 has been substituted by any amino acid, preferably by threonine (T), asparagine (N), serine(S), or glutamine (Q), more preferably by threonine (T).

In some embodiments, an SHC enzyme described herein comprises an amino acid sequence in which the serine(S) corresponding to position 382 in SEQ ID NO: 1 has been substituted by any amino acid, preferably by threonine (T), asparagine (N), or glutamine (Q), more preferably by threonine (T).

In some embodiments, an SHC enzyme described herein comprises an amino acid sequence in which the isoleucine (I) corresponding to position 399 in SEQ ID NO: 1 has been substituted by any amino acid, preferably by valine (V), alanine (A), or glycine (G), leucine (L) more preferably by valine (V).

In some embodiments, an SHC enzyme described herein comprises an amino acid sequence in which the tyrosine (Y) corresponding to position 483 in SEQ ID NO: 1 has been substituted by any amino acid, preferably by cysteine (C), methionine (M), or proline (P), more preferably by cysteine (C).

In some embodiments, an SHC enzyme described herein comprises an amino acid sequence in which the leucine (L) corresponding to position 539 in SEQ ID NO: 1 has been substituted by any amino acid, preferably by histidine (H), arginine (R), or lysine (K), more preferably by histidine (H).

In some embodiments, an SHC enzyme described herein comprises an amino acid sequence in which the glutamic acid (E) corresponding to position 585 in SEQ ID NO: 1 has been substituted by any amino acid, preferably by alanine (A), valine (V), isoleucine (I), glycine (G), or leucine (L), more preferably by alanine (A).

In some embodiments, a preferred SHC enzyme as described herein compres an amino acid sequence having at least 30%, 40%, 50%, 60%, or 70%, preferably at least 70%, identity or similarity with the sequence of SEQ ID NO: 1, preferably wherein the SHC enzyme comprises one or more amino acid substitutions relative to SEQ ID NO: 1 at one or more positions corresponding to position 2, 5, 35, 116, 166, 211, 212, 317, 355, 382, 399, 483, 539, and 585, preferably 2, 5, 35, 166, 211, 212, 355, 483, and 539, more preferably 2, 5, 35, 166, 211, 212, 483, and 539, most preferably 2, 5, 35, 166, 211, 483, and 539 in SEQ ID NO: 1. In some embodiments, the identity or similarity with the sequence of SEQ ID NO: 1 is at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, or 100%.

In some embodiments, an SHC enzyme described herein comprises an amino acid substitution relative to SEQ ID NO: 1 selected from the following:
(i) an asparagine (N), serine(S), threonine (T), or glutamine (Q) residue at a position corresponding to position 2 in SEQ ID NO: 1;
(ii) a proline (P), methionine (M), or cysteine (C) residue at a position corresponding to position 5 in SEQ ID NO: 1;
(iii) an alanine (A), isoleucine (I), valine (V), glycine (G), or leucine (L) residue at a position corresponding to position 35 in SEQ ID NO: 1;
(iv) a threonine (T), asparagine (N), serine(S), or glutamine (Q) residue at a position corresponding to position 116 in SEQ ID NO: 1;
(v) an alanine (A), isoleucine (I), valine (V), glycine (G), or leucine (L) residue at a position corresponding to position 166 in SEQ ID NO: 1;
(vi) a valine (V), alanine (A), isoleucine (I), glycine (G), or leucine (L) residue at a position corresponding to position 211 in SEQ ID NO: 1;
(vii) an arginine (R), lysine (K), or histidine (H) residue at a position corresponding to position 212 in SEQ ID NO: 1;
(viii) a methionine (M), proline (P), or cysteine (C) residue at a position corresponding to position 317 in SEQ ID NO: 1;
(ix) a threonine (T), asparagine (N), serine(S), or glutamine (Q) residue at a position corresponding to position 355 in SEQ ID NO: 1;
(x) a threonine (T), asparagine (N), or glutamine (Q) residue at a position corresponding to position 382 in SEQ ID NO: 1;
(xi) a valine (V), alanine (A), glycine (G), or leucine (L) at a position corresponding to position 399 in SEQ ID NO: 1;
(xii) a cysteine (C), methionine (M), or proline (P) residue at a position corresponding to position 483 in SEQ ID NO: 1;
(xiii) a histidine (H), arginine (R), or lysine (K) residue at a position corresponding to position 539 in SEQ ID NO: 1;
(xiv) an alanine (A), valine (V), isoleucine (I), glycine (G), or leucine (L) residue at a position corresponding to position 585 in SEQ ID NO: 1; or
(xv) any combination thereof.

In some embodiments, an SHC enzyme described herein comprises an amino acid substitution relative to SEQ ID NO: 1 selected from the following:
(i) an asparagine (N) residue at a position corresponding to position 2 in SEQ ID NO: 1;
(ii) a proline (P) residue at a position corresponding to position 5 in SEQ ID NO: 1;
(iii) an alanine (A) residue at a position corresponding to position 35 in SEQ ID NO: 1;
(iv) an threonine (T) residue at a position corresponding to position 116 in SEQ ID NO: 1;
(v) an alanine (A) residue at a position corresponding to position 166 in SEQ ID NO: 1;
(vi) a valine (V) residue at a position corresponding to position 211 in SEQ ID NO: 1;
(vii) an arginine (R) residue at a position corresponding to position 212 in SEQ ID NO: 1;
(viii) a methionine (M) residue at a position corresponding to position 317 in SEQ ID NO: 1;
(ix) a threonine (T) residue at a position corresponding to position 355 in SEQ ID NO: 1;
(x) a threonine (T) residue at a position corresponding to position 382 in SEQ ID NO: 1;
(xi) a valine (V) residue at a position corresponding to position 399 in SEQ ID NO: 1;
(xii) a cysteine (C) residue at a position corresponding to position 483 in SEQ ID NO: 1;
(xiii) a histidine (H) residue at a position corresponding to position 539 in SEQ ID NO: 1;
(xiv) an alanine (A) residue at a position corresponding to position 585 in SEQ ID NO: 1; or
(xv) any combination thereof.

In some embodiments, an SHC enzyme described herein comprises an amino acid substitution relative to SEQ ID NO: 1 selected from the following corresponding positions in SEQ ID NO: 1:
(i) 2, 35, 355, and 539;
(ii) 166;
(iii) 2 and 483;
(iv) 2, 483, and 539;
(v) 2, 5, 35, 539;
(vi) 2, 5, 35, and 483;
(vii) 2, 5, 35, 166, and 539;
(viii) 2, 5, 35, 166, 211, and 539
(ix) 2, 5, 35, 211, 212, 483, and 539
(x) 2, 166, and 483;
(xi) 2, 166, 483, and 539;
(xii) 2, 166, 211, and 483; or
(xiii) 2, 166, 211, 483, and 539.

In some embodiments, an SHC enzyme described herein comprises an amino acid substitution relative to SEQ ID NO: 1 selected from the following:
(i) I2N, T35A, A355T, and L539H;
(ii) T166A;
(iii) I2N and Y483C;
(iv) I2N, Y483C, and L539H;
(v) I2N, L5P, T35A, L539H;
(vi) I2N, L5P, T35A, and Y483C;
(vii) I2N, L5P, T35A, T166A, and L539H;
(viii) I2N, L5P, T35A, T166A, E211V, and L539H
(ix) I2N, L5P, T35A, E211V, S212R, Y483C, and L539H
(x) I2N, T166A, and Y483C;
(xi) I2N, T166A, Y483C, and L539H;
(xii) I2N, T166A, E211V, and Y483C; or
(xiii) I2N, T166A, E211V, Y483C, and L539H.

In some embodiments, an SHC enzyme described herein comprises the following amino acid substitutions relative to SEQ ID NO: 1: I2N, T35A, A355T, L539H. Optionally, it further comprises an E211V substitution relative to SEQ ID NO: 1.

In some embodiments, an SHC enzyme described herein comprises the following amino acid substitution relative to SEQ ID NO: 1: T166A. Optionally, it further comprises an E211V and/or an L539H substitution relative to SEQ ID NO: 1.

In some embodiments, an SHC enzyme described herein comprises the following amino acid substitutions relative to SEQ ID NO: 1: I2N, Y483C. Optionally, it further comprises an E211V and/or an L539H substitution relative to SEQ ID NO: 1.

In some embodiments, an SHC enzyme described herein comprises the following amino acid substitutions relative to SEQ ID NO: 1: I2N, Y483C, L539H. Optionally, it further comprises an E211V substitution relative to SEQ ID NO: 1.

In some embodiments, an SHC enzyme described herein comprises the following amino acid substitutions relative to SEQ ID NO: 1: I2N, L5P, T35A, L539H. Optionally, it further comprises an E211V substitution relative to SEQ ID NO: 1.

In some embodiments, an SHC enzyme described herein comprises the following amino acid substitutions relative to SEQ ID NO: 1: I2N, L5P, T35A, Y483C. Optionally, it further comprises an E211V and/or an L539H substitution relative to SEQ ID NO: 1.

In some embodiments, an SHC enzyme described herein comprises the following amino acid substitutions relative to SEQ ID NO: 1: I2N, L5P, T35A, T166A, L539H. Optionally, it further comprises an E211V substitution relative to SEQ ID NO: 1.

In some embodiments, an SHC enzyme described herein comprises the following amino acid substitutions relative to SEQ ID NO: 1: I2N, L5P, T35A, T166A, E211V, L539H.

In some embodiments, an SHC enzyme described herein comprises the following amino acid substitutions relative to SEQ ID NO: 1: I2N, L5P, T35A, E211V, S212R, Y483C, L539H.

In some embodiments, an SHC enzyme described herein comprises the following amino acid substitutions relative to SEQ ID NO: 1: I2N, T166A, Y483C. Optionally, it further comprises an E211V and/or an L539H substitution relative to SEQ ID NO: 1.

In some embodiments, an SHC enzyme described herein comprises the following amino acid substitutions relative to SEQ ID NO: 1: I2N, T166A, Y483C, L539H. Optionally, it further comprises an E211V substitution relative to SEQ ID NO: 1.

In some embodiments, an SHC enzyme described herein comprises the following amino acid substitutions relative to SEQ ID NO: 1: I2N, T166A, E211V, Y483C. Optionally, it further comprises an L539H substitution relative to SEQ ID NO: 1.

In some embodiments, an SHC enzyme described herein comprises the following amino acid substitutions relative to SEQ ID NO: 1: I2N, T166A, E211V, Y483C, L539H.

In some embodiments, an SHC enzyme described herein comprises the following amino acid substitutions relative to SEQ ID NO: 1: I2N, T166A. Optionally, it further comprises an E211V and/or an L539H substitution relative to SEQ ID NO: 1. Optionally, it further comprises a Y483C substitution relative to SEQ ID NO: 1.

In some embodiments, any of the SHC enzymes described herein further comprise one or more substitutions relative to SEQ ID NO: 1 selected from L5P, T35A, E211V, Y483C, and L539H.

The skilled person understands that the numbering of positions denoting the amino acid substitutions described herein refers to the corresponding positions in SEQ ID NO: 1, as discussed elsewhere herein.

In some embodiments, any of the SHC enzymes described herein comprises an amino acid sequence that is at least 90% identical to any one of SEQ ID NOs: 4, 6, 8, 10, 12, 14, 16, 18, 20, 22, 24, 26, 28, 30, 32, 34, 36, 38, 40 or 42, preferably SEQ ID NOs: 4, 8, 18, 20, 22, 24, 30, 32, 34, 36, 38, 40 or 42, more preferably SEQ ID NOs: 30, 32, 34, 36, 38, 40 or 42, most preferably SEQ ID NOs: 30, 38, 40 or 42. In some embodiments, any of the SHC enzymes described herein comprises an amino acid sequence that is at least 90% identical to any one of SEQ ID NOs: 30, 34, 36, 40 or 42. In some embodiments, any of the SHC enzymes described herein comprises an amino acid sequence that is at least 90% identical to SEQ ID NO: 4. In some embodiments, any of the SHC enzymes described herein comprises an amino acid sequence that is at least 90% identical to SEQ ID NO: 6. In some embodiments, any of the SHC enzymes described herein comprises an amino acid sequence that is at least 90% identical to SEQ ID NO: 8. In some embodiments, any of the SHC enzymes described herein comprises an amino acid sequence that is at least 90% identical to SEQ ID NO: 10. In some embodiments, any of the SHC enzymes described herein comprises an amino acid sequence that is at least 90% identical to SEQ ID NO: 12. In some embodiments, any of the SHC enzymes described herein comprises an amino acid sequence that is at least 90% identical to SEQ ID NO: 14. In some embodiments, any of the SHC enzymes described herein comprises an amino acid sequence that is at least 90% identical to SEQ ID NO: 16. In some embodiments, any of the SHC enzymes described herein comprises an amino acid sequence that is at least 90% identical to SEQ ID NO: 18. In some embodiments, any of the SHC enzymes described herein comprises an amino acid sequence that is at least 90% identical to SEQ ID NO: 20. In some embodiments, any of the SHC enzymes described herein comprises an amino acid sequence that is at least 90% identical to SEQ ID NO: 22. In some embodiments, any of the SHC enzymes described herein comprises an amino acid sequence that is at least 90% identical to SEQ ID NO: 24. In some embodiments, any of the SHC enzymes described herein comprises an amino acid sequence that is at least 90% identical to SEQ ID NO: 26. In some embodiments, any of the SHC enzymes described herein comprises an amino acid sequence that is at least 90% identical to SEQ ID NO: 28. In some embodiments, any of the SHC enzymes described herein comprises an amino acid sequence that is at least 90% identical to SEQ ID NO: 30. In some embodiments, any of the SHC enzymes described herein comprises an amino acid sequence that is at least 90% identical to SEQ ID NO: 32. In some embodiments, any of the SHC enzymes described herein comprises an amino acid sequence that is at least 90% identical to SEQ ID NO: 34. In some embodiments, any of the SHC enzymes described herein comprises an amino acid sequence that is at least 90% identical to SEQ ID NO: 36. In some embodiments, any of the SHC enzymes described herein comprises an amino acid sequence that is at least 90% identical to SEQ ID NO: 38. In some embodiments, any of the SHC enzymes described herein comprises an amino acid sequence that is at least 90% identical to SEQ ID NO: 40. In some embodiments, any of the SHC enzymes described herein comprises an amino acid sequence that is at least 90% identical to SEQ ID NO: 42. The amino acid sequence may be at least 91% identical. The amino acid sequence may be at least 92% identical. The amino acid sequence may be at least 93% identical. The amino acid sequence may be at least 94% identical. The amino acid sequence may be at least 95% identical. The amino acid sequence may be at least 95.5% identical. The amino acid sequence may be at least 96% identical. The amino acid sequence may be at least 96.5% identical. The amino acid sequence may be at least 97% identical. The amino acid sequence may be at least 97.5% identical. The amino acid sequence may be at least 98% identical. The amino acid sequence may be at least 98.5% identical. The amino acid sequence may be at least 99% identical. The amino acid sequence may be at least 99.5% identical. The amino acid sequence may be identical.

In some embodiments, any of the SHC enzymes described herein is encoded by a nucleic acid comprising a nucleotide sequence that is at least 90% identical to any one of SEQ ID NOs: 3, 5, 7, 9, 11, 13, 15, 17, 19, 21, 23, 25, 27, 29, 31, 33, 35, 37, 39 or 41, preferably SEQ ID NOs: 3, 7, 17, 19, 21, 23, 29, 31, 33, 35, 37, 39 or 41, more preferably SEQ ID NOs: 29, 31, 33, 35, 37, 39 or 41, most preferably SEQ ID NOs: 29, 37, 39 or 41. In some embodiments, any of the SHC enzymes described herein is encoded by a nucleic acid comprising a nucleotide sequence that is at least 90% identical to any one of SEQ ID NOs: 29, 33, 35, 39 or 41. In some embodiments, any of the SHC enzymes described herein is encoded by a nucleic acid comprising a nucleotide sequence that is at least 90% identical to SEQ ID NO: 3. In some embodiments, any of the SHC enzymes described herein is encoded by a nucleic acid comprising a nucleotide sequence that is at least 90% identical to SEQ ID NO: 5. In some embodiments, any of the SHC enzymes described herein is encoded by a nucleic acid comprising a nucleotide sequence that is at least 90% identical to SEQ ID NO: 7. In some embodiments, any of the SHC enzymes described herein is encoded by a nucleic acid comprising a nucleotide sequence that is at least 90% identical to SEQ ID NO: 9. In some embodiments, any of the SHC enzymes described herein is encoded by a nucleic acid comprising a nucleotide sequence that is at least 90% identical to SEQ ID NO: 11. In some embodiments, any of the SHC enzymes described herein is encoded by a nucleic acid comprising a nucleotide sequence that is at least 90% identical to SEQ ID NO: 13. In some embodiments, any of the SHC enzymes described herein is encoded by a nucleic acid comprising a nucleotide sequence that is at least 90% identical to SEQ ID NO: 15. In some embodiments, any of the SHC enzymes described herein is encoded by a nucleic acid comprising a nucleotide sequence that is at least 90% identical to SEQ ID NO: 17. In some embodiments, any of the SHC enzymes described herein is encoded by a nucleic acid comprising a nucleotide sequence that is at least 90% identical to SEQ ID NO: 19. In some embodiments, any of the SHC enzymes described herein is encoded by a nucleic acid comprising a nucleotide sequence that is at least 90% identical to SEQ ID NO: 21. In some embodiments, any of the SHC enzymes described herein is encoded by a nucleic acid comprising a nucleotide sequence that is at least 90% identical to SEQ ID NO: 23. In some embodiments, any of the SHC enzymes described herein is encoded by a nucleic acid comprising a nucleotide sequence that is at least 90% identical to SEQ ID NO: 25. In some embodiments, any of the SHC enzymes described herein is encoded by a nucleic acid comprising a nucleotide sequence that is at least 90% identical to SEQ ID NO: 27. In some embodiments, any of the SHC enzymes described herein is encoded by a nucleic acid comprising a nucleotide sequence that is at least 90% identical to SEQ ID NO: 29. In some embodiments, any of the SHC enzymes described herein is encoded by a nucleic acid comprising a nucleotide sequence that is at least 90% identical to SEQ ID NO: 31. In some embodiments, any of the SHC enzymes described herein is encoded by a nucleic acid comprising a nucleotide sequence that is at least 90% identical to SEQ ID NO: 33. In some embodiments, any of the SHC enzymes described herein is encoded by a nucleic acid comprising a nucleotide sequence that is at least 90% identical to SEQ ID NO: 35. In some embodiments, any of the SHC enzymes described herein is encoded by a nucleic acid comprising a nucleotide sequence that is at least 90% identical to SEQ ID NO: 37. In some embodiments, any of the SHC enzymes described herein is encoded by a nucleic acid comprising a nucleotide sequence that is at least 90% identical to SEQ ID NO: 39. In some embodiments, any of the SHC enzymes described herein is encoded by a nucleic acid comprising a nucleotide sequence that is at least 90% identical to SEQ ID NO: 41.

The nucleotide sequence may be at least 91% identical. The nucleotide sequence may be at least 92% identical. The nucleotide sequence may be at least 93% identical. The nucleotide sequence may be at least 94% identical. The nucleotide sequence may be at least 95% identical. The nucleotide sequence may be at least 95.5% identical. The nucleotide sequence may be at least 96% identical. The nucleotide sequence may be at least 96.5% identical. The nucleotide sequence may be at least 97% identical. The nucleotide sequence may be at least 97.5% identical. The nucleotide sequence may be at least 98% identical. The nucleotide sequence may be at least 98.5% identical. The nucleotide sequence may be at least 99% identical. The nucleotide sequence may be at least 99.5% identical. The nucleotide sequence may be identical.

As used herein, the term “activity” or “enzymatic activity” or “biological activity” refers to the ability of an enzyme to react with a substrate to provide a target product. “SHC activity” or “SHC enzymatic activity” or “SHC biological activity” may, for example, refer to the ability of an SHC enzyme described herein to convert a compound of formula (II) to a compound of formula (I), for example their ability to convert hydroxyfarnesylacetone to (+)-amberketal. It may also, for example, refer to the ability of an SHC enzyme described herein to convert a compound of formula (IIa) to a compound of formula (Ia), preferably to a compound of formula (V). It may also, for example, refer to the ability of an SHC enzyme described herein to convert a compound of formula (II) to a compound of formula (I) and/or a compound of formula (IIa) to a compound of formula (Ia) (such as a compound of formula (V)), wherein the compound of formula (II) and the compound of formula (IIa) are comprised in a mixture, as described earlier herein. An SHC enzyme exhibiting its enzymatic activity may also be referred to herein as a functional enzyme. Enzymatic activity can be determined, for example, using what is known as an activity test via the monitoring of the increase of a target product, the decrease of the substrate (or starting material) or via a combination of these parameters as a function of time.

An SHC enzyme described herein may, for example, have increased enzymatic activity for the conversion of a compound of formula (II) (e.g., hydroxyfarnesylacetone) to a compound of formula (I) (e.g., (+)-amberketal) and/or increased enzymatic activity for the conversion of a compound of formula (IIa) to a compound of formula (Ia) (such as a compound of formula (V)) compared to its parent SHC enzyme. Increased enzymatic activity may refer to any aspect of the enzymatic conversion of the compound of formula (II) to the compound of formula (I) and/or of the compound of formula (IIa) to the compound of formula (Ia) (such as the compound of formula (V)) including, for example, increased total conversion (yield), increased rate of conversion (e.g. (but not limited to), in the first 4 hours, or first 6 hours, or in the first 12 hours, or in the first 24 hours, or in the first 48 hours, or in the first 72 hours, or in the first 96 hours, or in the first 120 hours, or in the first 144 hours, or in the first 168 hours of reaction), increased production of the compound of formula (I) and/or the compound of formula (Ia) (such as the compound of formula (V)), and/or decreased production of by-products. Increased enzymatic activity may be defined by increased productivity in general, which may be defined in terms of compound of formula (I) and or compound of formula (Ia) (such as compound of formula (V)) produced per hour of reaction time (typically measured from the time point of the reaction start), per gram of biocatalyst and per litre of reaction.

In some embodiments, utilization of an SHC enzyme according to the methods described herein results in at least 5%, at least 10%, at least 20%, at least 30%, at least 40%, at least 50%, at least 60%, at least 70%, at least 80%, at least 90%, at least 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold higher productivity as compared to utilization of its parent SHC enzyme.

Assays for determining and quantifying SHC enzymatic activity are known in the art and further examples are provided in the experimental section herein. By way of example, activity of an SHC enzyme described herein can be determined by incubating purified enzyme(s) or extracts from host cells or a complete recombinant host cell that has produced the enzyme(s) with an appropriate substrate under appropriate conditions and carrying out an analysis of the substrate and reaction products (e.g. by gas chromatography (GC) or HPLC analysis, as discussed in standard handbooks in the art such as the Encyclopedia of Analytical Science: 3^{rd} Edition (supra)). Further details on SHC enzymatic activity assays and analysis of the reaction products are provided in the Examples. These assays may include producing the enzymes in recombinant host cells (e.g. E. coli).

An SHC enzyme described herein may, for example, provide increased total conversion of a compound of formula (II) compared to its parent SHC enzyme. Therefore, a method using an SHC enzyme described herein may have an increased total conversion of a compound of formula (II) compared to the method using its parent SHC enzyme. An SHC enzyme described herein may, for example, provide increased total conversion of a compound of formula (IIa) compared to its parent SHC enzyme. Therefore, a method using an SHC enzyme described herein may have an increased total conversion of a compound of formula (IIa) compared to the method using its parent SHC enzyme. An SHC enzyme described herein may, for example, provide increased total conversion of a mixture comprising a compound of formula (II) and a compound of formula (IIa) compared to its parent SHC enzyme. Therefore, a method using an SHC enzyme described herein may result in an increased total conversion of a compound of formula (II) and/or of a compound of formula (IIa) compared to a method using its parent SHC enzyme, wherein the compound of formula (II) and the compound of formula (IIa) are comprised in a mixture as described earlier herein.

An SHC enzyme described herein may, for example, provide increased rate of a compound of formula (II) and/or of a compound of formula (IIa) conversion compared to its parent SHC enzyme. Therefore, a method using an SHC enzyme described herein may have an increased rate of compound of formula (II) and/or of a compound of formula (IIa) conversion compared to the method using its parent SHC enzyme. The SHC enzyme may, for example, provide increased rate of compound of formula (II) and/or of compound of formula (IIa) conversion over the first 2 hours, over the first 4 hours, over the first 6 hours, over the first 8 hours, over the first 12 hours, over the first 24 hours, over the first 36 hours, over the first 48 hours, over the first 72 hours, over the first 96 hours, over the first 120 hours, over the first 144 hours, or over the first 168 hours of the reaction compared to the parent SHC enzyme. Therefore, a method using an SHC enzyme described herein may have an increased rate of compound of formula (II) and/or of compound formula (IIa) conversion over the first 2 hours, over the first 4 hours, over the first 6 hours, over the first 8 hours, over the first 12 hours, over the first 24 hours, over the first 36 hours, over the first 48 hours, over the first 72 hours, over the first 96 hours, over the first 120 hours, over the first 144 hours, or over the first 168 hours, preferably over the first 24 hours, of the reaction compared to a method using its parent SHC enzyme.

In some embodiments, the total conversion and/or rate of a compound of formula (II) and/or of compound of formula (IIa) conversion exhibited by an SHC enzyme described herein is at least 5%, at least 10%, at least 20%, at least 30%, at least 40%, at least 50%, at least 60%, at least 70%, at least 80%, at least 90%, at least 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold higher as compared to its parent SHC enzyme.

In some embodiments, the improvement in total conversion and/or rate of compound of formula (II) and/or of compound of formula (IIa) conversion, exhibited by an SHC enzyme described herein as compared to its parent SHC enzyme, is obtained in mixtures comprising a compound of formula (II) and a compound of formula (IIa) as described herein.

An SHC enzyme described herein may, for example, provide improved conversion of a compound of formula (II) to a compound of formula (I) compared to its parent SHC enzyme, which may alternatively be defined as the yield of a compound of formula (I). In other words, an SHC enzyme described herein may result in more grams/moles of a compound of formula (I) being formed per gram/mole of compound of formula (II) that is converted compared to its parent SHC enzyme. An SHC enzyme described herein may, for example, provide improved conversion of a compound of formula (IIa) to a compound of formula (Ia) (such as a compound of formula (V)) compared to its parent SHC enzyme, which may alternatively be defined as the yield of a compound of formula (Ia). In other words, an SHC enzyme described herein may result in more grams/moles of a compound of formula (Ia) (such as a compound of formula (V)) being formed per gram/mole of compound of formula (IIa) that is converted compared to its parent SHC enzyme.

In some embodiments, the conversion of a compound of formula (II) to a compound of formula (I) and/or of a compound of formula (IIa) to a compound of formula (Ia) (such as a compound of formula (V)) achieved by an SHC enzyme described herein is at least 5%, at least 10%, at least 20%, at least 30%, at least 40%, at least 50%, at least 60%, at least 70%, at least 80%, at least 90%, at least 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold higher as compared to its parent SHC enzyme.

In some embodiments, an SHC enzyme described herein achieves a conversion of a compound of formula (II) to a compound of formula (I) and/or of a compound of formula (IIa) to a compound of formula (Ia) (such as a compound of formula (V)) of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, given in mol percent and based on the mols of compound of formula (II) employed. Preferably, the yield is from 5 to 100, from 10 to 100, from 20 to 100, from 30 to 100, from 35 to 100, more preferably from 35 to 100, from 45 to 100, from 50 to 100, from 60 to 100, or from 70 to 100 mol percent. Preferably, the conversion is measured at or after 24 hours of reaction time.

In some embodiments, the improvement in conversion of a compound of formula (II) to a compound of formula (I) and/or of a compound of formula (IIa) to a compound of formula (Ia) (such as a compound of formula (V)), exhibited by an SHC enzyme described herein as compared to its parent SHC enzyme as described above, is obtained in mixtures comprising a compound of formula (II) and a compound of formula (IIa) as described herein. On-limiting additional parameters that may characterize an SHC enzyme described herein are: specificity (e.g., substrate specificity, bond specificity, group specificity, optical specificity, co-factor specificity, geometric specificity), reaction rate, by-product formation, and sensitivity to reaction conditions (e.g., pH, temperature, substrate concentration, concentration of solubilizing agents such as SDS), resistance to product inhibition, among others.

An SHC enzyme described herein may be compared with its parent enzyme under the same reaction conditions (e.g., same pH, temperature, substrate concentration, concentration of solubilizing agents such as SDS) or under conditions that have been individually defined as optimal for the activity of each enzyme and which may be the same or different to each other. The reaction performance of an SHC enzyme in relation to any of the reaction conditions as compared its parent SHC enzyme may be assessed using any of the abovementioned parameters, such as productivity, total conversion or increased rate of a compound of formula (II) and/or of a compound of formula (IIa) conversion, or yield of a compound of formula (I) and/or a compound of formula (Ia) (such as a compound of formula (V)), and may be improved, for example, by at least 5%, 10%, 20%, 30%, 40%, 50%, 60%, 70%, 80%, 90%, 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold. Preferably, the reaction performance is measured at or after 24 hours of reaction time.

The reaction performance of an SHC enzyme as described herein may be assessed using any substrate concentration, for example, a substrate concentration of at least 1 g/L or higher. In embodiments wherein host cells expressing SHC enzymes described herein are utilized, the reaction performance may be assessed using any substrate concentration as defined above and/or any cell concentration, for example, a cell concentration of at least 1 g/L or higher.

In particular, an SHC enzyme described herein may exhibit improved reaction performance at a high substrate concentration as compared to its parent SHC enzyme. A compound of formula (II) concentration of 50 g/L or higher may be considered a high substrate concentration. In some embodiments, the SHC enzyme may exhibit improved reaction performance at a compound of formula (II) concentration of 50 g/L or higher, 60 g/L or higher, 70 g/L or higher, 80 g/L or higher, 90 g/L or higher, 100 g/L or higher, 110 g/L or higher, 120 g/L or higher, 130 g/L or higher, 135 g/L or higher, 150 g/L or higher, 175 g/L or higher, or 200 g/L or higher, or 250 g/L or higher, preferably at a concentration of 135 g/L or higher, as compared to its parent SHC enzyme.

In some embodiments wherein host cells expressing SHC enzymes as described herein are utilized, an SHC enzyme may exhibit improved reaction performance at a high cell concentration as compared to its parent SHC enzyme. A cell concentration of 50 g/L or higher may be considered a high cell concentration. The SHC enzyme may exhibit improved reaction performance at a cell concentration of 50 g/L or higher, 60 g/L or higher, 70 g/L or higher, 80 g/L or higher, 90 g/L or higher, 100 g/L or higher, 110 g/L or higher, 120 g/L or higher, 130 g/L or higher, 150 g/L or higher, 175 g/L or higher, or 200 g/L or higher, or 250 g/L or higher, preferably 175 g/L or higher as compared to its parent SHC enzyme.

In some embodiments, the improvement in reaction performance exhibited by an SHC enzyme described herein as compared to its parent SHC enzyme is obtained in mixtures comprising a compound of formula (II) and a compound of formula (IIa) as described herein.

In some embodiments, the ratio of SHC enzyme to substrate or the ratio of host cell expressing the SHC enzyme to substrate may be adjusted to optimize the bioconversion reaction.

In some embodiments, the SHC enzyme or the host cell expressing the SHC enzyme has a weight ratio to the substrate of 0.1-4 to 1 or of about 0.1-4 to 1 (0.1-4:1), 0.1-3 to 1 or of about 0.1-3 to 1 (0.1-3:1), 0.1-2 to 1 or of about 0.1-2 to 1 (0.1-2:1), of 0.25-2 to 1 or of about 0.25-2 to 1 (0.25-2:1), of 0.5-2 to 1 or of about 0.5-2 to 1 (0.5-2:1), of 0.1 to 1 or of about 0.1 to 1 (0.1:1), of 0.5 to 1 or of about 0.5 to 1 (0.5:1), of 1 to 1 or of about 1 to 1 (1:1), of 1.5 to 1 or of about 1.5 to 1 (1.5:1), or of 2 to 1 or of about 2 to 1 (2:1), preferably of 0.1 to 1 or of about 0.1 to 1 (0.1:1), of 0.5 to 1 or of about 0.5 to 1 (0.5:1), or of 1 to 1 or of about 1 to 1 (1:1).

Accordingly, an SHC enzyme described herein may exhibit at least one, at least two, at least three, or all of the following benefits as compared to its parent SHC enzyme:
Improved conversion rate of a compound of formula (II) and/or of a compound of formula (IIa)
Improved yield of a compound of formula (I) and/or a compound of formula (Ia)
Improved reaction performance (e.g., conversion rate, productivity, yield at high substrate concentration

As used herein, “selectivity” of an SHC enzyme described herein may refer to the ability of the enzyme to react with a particular substrate compared to another substrate. As a non-limiting example, an SHC enzyme may be selective for the E,Z-isomer of a compound of formula (II) in comparison to the E,E-isomer or another isomer, meaning that the enzyme is more likely to convert the E,Z-isomer than the E,E-isomer or another isomer. As another non-limiting example, an SHC enzyme may be selective for the E,Z-isomer of a compound of formula (IIa) in comparison to the E,E-isomer or another isomer. As another non-limiting example, an SHC enzyme may be selective for a particular constitutional isomer of a compound, for example a compound of formula (II) or a compound of formula (IIa). As another non-limiting example, SHC enzymes described and used in the methods described herein may, for instance, have a selectivity equal to or greater than 75% or about 75% for a compound of formula (II). As further non-limiting examples, the SHC enzyme or its parent SHC enzyme may have a selectivity equal to or greater than 80% or about 80%, equal to or greater than 85% or about 85%, equal to or greater than 90% or about 90%, equal to or greater than 95% or about 95%. For example, the SHC enzyme or its parent SHC enzyme may have a selectivity up to 100% or about 100%, for example less than 100% or about 100%, such as equal to or less than 99.5% or about 99.5%, equal to or less than 99% or about 99%, equal to or less than 98% or about 98%, or equal to or less than 97% or about 97%.

As another non-limiting example, SHC enzymes described and used in the methods described herein may, for instance, have a selectivity equal to or greater than 75% or about 75% for a compound of formula (IIa). As further non-limiting examples, the SHC enzyme or its parent SHC enzyme may have a selectivity equal to or greater than 80% or about 80%, equal to or greater than 85% or about 85%, equal to or greater than 90% or about 90%, equal to or greater than 95% or about 95%. For example, the SHC enzyme or its parent SHC enzyme may have a selectivity up to 100% or about 100%, for example less than 100% or about 100%, such as equal to or less than 99.5% or about 99.5%, equal to or less than 99% or about 99%, equal to or less than 98% or about 98%, or equal to or less than 97% or about 97%.

The methods for making the compound of formula (I) and/or the compound of formula (Ia) (such as the compound of formula (V)) disclosed herein may be carried out at an optimum temperature range or optimum temperature and/or optimum pH range or optimum pH and/or solubilizing agent (such as SDS) optimum concentration range or optimum solubilizing agent (such as SDS) concentration for the specific enzyme used (such as a particular SHC variant), as discussed later herein. Examples are further provided in the experimental section. Additional examples may be found in WO2021/209482.

### Nucleic Acids and Vectors

The SHC enzymes described herein may be encoded by a nucleotide sequence. The nucleic acid molecule comprising the nucleotide sequence may, for example, be an isolated nucleic acid molecule. Accordingly, the disclosure further provides a nucleic acid molecule comprising a nucleotide sequence encoding a squalene hopene cyclase (SHC) enzyme as described herein.

The terms “nucleic acid” or “nucleic acid molecule” as used herein are interchangeable and refer to polynucleotides of the disclosure which can be DNA, cDNA, genomic DNA, synthetic DNA, or RNA, and can be double-stranded or single-stranded, a sense or an antisense strand.

The terms particularly apply to a polynucleotide encoding an SHC enzyme described herein, e.g., a full-length nucleotide sequence or fragment thereof, which encodes an SHC polypeptide or fragment thereof exhibiting its enzymatic activity. The terms also include a separate molecule such as a cDNA wherein its corresponding genomic DNA has introns and therefore a different sequence, a genomic fragment that lacks at least one of the flanking genes, a fragment of cDNA or genomic DNA produced by polymerase chain reaction (PCR) and that lacks at least one of the flanking genes, a restriction fragment that lacks at least one of the flanking genes, and a nucleic acid which is a degenerate variant of a cDNA or a naturally occurring nucleic acid.

A nucleic acid molecule may comprise a codon-optimised sequence for expression in a particular host cell. “Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host cell. For example, to suit the codon preference of mammalian, insect, plant, or microbial cells, preferably microbial cells, such as E. coli, and others. Examples of microbial cells include eukaryotes such as yeasts, filamentous fungi, and algae, and prokaryotes such as bacteria and archaea. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs).

In this regard, a nucleic acid molecule encoding an SHC enzyme may comprise the original nucleotide sequence as found in the source organism or may comprise a codon-optimized sequence for expression in a selected host cell, such as E. coli, and others.

The disclosure further provides a nucleic acid construct comprising a nucleotide sequence encoding an SHC enzyme as described herein, operably linked to a regulatory sequence, for example a transcription inititiation sequence such as a promoter sequence. A “nucleic acid construct” as used herein refers to an artificially created nucleic acid which typically is to be introduced to a target cell. Thus, a regulatory sequence that is operably linked to the nucleotide sequence encoding an SHC enzyme as described herein may not be associated with it in nature.

Optionally, other regulatory sequences such as transcription terminators, enhancers, repressors, silencers, kozak sequences, polyA sequences, and the like may be operably linked to the nucleotide sequence encoding an SHC enzyme.

The regulatory sequences referred to above include but are not limited to inducible and non-inducible, constitutive, cell-cycle regulated, metabolically regulated, enhancers, operators, silencers, repressors and other elements that are known to those skilled in the art and that drive or otherwise regulate gene expression in a cell. Such regulatory sequences include but are not limited to regulatory sequences directing constitutive expression or which allow inducible expression such as, for example, the CUP-1 promoter, the Tet-repressor as employed, for example, in the Tet-on or Tet-off systems, the Lac operon regulatory sequences, or the Trp operon regulatory sequences.

As a non-limiting example, when the Lac operon regulatory sequences are operably linked to a nucleotide sequence of interest, isopropyl β-D-1-thiogalactopyranoside (IPTG) is an effective inducer of gene expression in the concentration range of e.g., 100 pM to 1.0 mM. This compound is a molecular mimic of allolactose, a lactose metabolite that triggers transcription of the Lac operon, and may, therefore, be used to induce nucleotide sequence expression when the nucleotide sequence is under the control of the Lac operator.

The nucleic acid constructs described herein may further comprise a nucleotide sequence encoding an additional polypeptide, for example, a sequence that functions as a marker or reporter, and/or a sequence that enables the isolation and/or purification (e.g., via affinity chromatography) of the encoded polypeptide, such as a tag (for example a His-tag), and the like. In this regard, the nucleic acid construct may comprise a nucleotide sequence that encodes a “hybrid”, “fusion” or “chimeric” protein which represents a fusion of an SHC enzyme, for example, a marker, reporter, or a tag. Fusion proteins can comprise one or more amino acids (such as but not limited to Histidine (His)), usually at the N-terminus of the protein but also at the C-terminus or fused within internal regions of the protein, compared to the SHC enzyme they originate from. Such fusion proteins or nucleic acid constructs encoding such proteins typically serve three purposes: (i) to increase production of recombinant proteins; (ii) to increase the solubility of the recombinant protein; and (iii) to aid in the isolation and/or purification of the recombinant protein by providing a ligand for affinity purification. An SHC enzyme described herein may be referred to as isolated when it is separated from the cellular or in vitro components used in its production.

A marker may be a selectable marker. The term “selectable marker” refers herein to a polypeptide that can be used for selection of host cells expressing it by conferring a selective advantage to said cells upon exposure to selective conditions. A selectable marker may enable positive or negative selection. Suitable selection markers are known in the art and such markers and selection methods are discussed e.g. in standard publications such as Mortensen and Kingston (2009) Curr Protoc Mol Biol 86:9.5.1-9.5. 13, incorporated herein by reference in its entirety, as well as standard handbooks such as Ausubel et al. (2003) and Sambrook and Green (2012) (supra). The skilled person understands that a specific selectable marker may enable positive or negative selection depending on the host cell and/or the selective conditions which are applied. Positive selectable markers are markers that enable growth of the host cell upon exposure to selective conditions wherein growth would otherwise not occur. Negative selectable markers are markers that prohibit growth of the host cell upon exposure to selective conditions. Non-limiting examples of suitable markers and reporter polypeptides that may be encoded by additional sequences comprised in the nucleotide construct include beta-lactamase, chloramphenicol acetyltransferase (CAT), adenosine deaminase (ADA), aminoglycoside phosphotransferase dihydrofolate reductase (DHFR), hygromycin-B-phosphotransferase (HPH), thymidine kinase (TK), beta-galactosidase, and xanthine guanine phosphoribosyltransferase (XGPRT).

Examples of suitable tags include AviTag, calmodulin-tag, polyglutamate-tag, E-tag, FLAG-tag, HA-tag, His-tag, Myc-tag, S-tag, SBP-tag, Softag 1 and 3, Strep-tag, TC-tag, V5-tag, VSV-tag, X-press tag, isopeptag, SpyTag, BCCP, glutathione-S-transferase-tag, GFP-tag, Halo-tag, maltose binding protein-tag, Nus-tag, thioredoxin-tag, and Fc-tag.

The skilled person is aware of suitable regulatory sequences and of additional sequences that may be comprised in a nucleic acid construct of the disclosure, as well as of molecular toolbox techniques that can be used to arrive at the nucleic acid constructs described herein, and examples may be found in standard handbooks such as Ausubel et al., Current Protocols in Molecular Biology, 3^{rd} edition, John Wiley & Sons Inc (2003) and in Sambrook and Green, Molecular Cloning. A Laboratory Manual, 4th Edition, Cold Spring Harbor Laboratory Press (2012); both of which are incorporated herein by reference in their entireties. Further examples may be found in WO2021/209482.

The disclosure further provides a vector comprising a nucleic acid molecule or a nucleic acid construct as described herein. As used herein, a “vector” is a nucleic acid molecule that is used as a vehicle to artificially carry foreign genetic material into a cell where it can be replicated and/or expressed. A vector may be linear or circular. A vector may be maintained in a host cell in a low-copy number (e.g. 1-2 copies per cell), a medium-copy number (e.g., 3-20 copies per cell), or a high-copy number (e.g., >20 copies per cell). The origins of replication of low-, medium-, and high-copy vectors are known to the skilled person. The vector may, for example, be a plasmid, a megaplasmid, a cosmid, a phagemid, a phage, a viral vector (e.g., an adenoviral or retroviral vector), a knock-out or knock-in construct, or an artificial chromosome such as a bacterial, yeast, plant, or mammalian artificial chromosome. A preferred vector is a plasmid. The skilled person understands that the terms nucleic acid construct and vector may overlap, for example, in the case of a plasmid.

It is preferred that the proteins encoded by a nucleic acid molecule, nucleic acid construct, or vector described herein are expressed upon their introduction to a host cell.

### Host Cells, Methods of Making Host Cells, and Methods of Making a Compound of Formula (I) Using Host Cells

In an aspect, the disclosure provides a host cell comprising a nucleic acid molecule, a nucleic acid construct, or a vector as described herein. A host cell preferably expresses (alternatively referred to herein as “produces”) an SHC enzyme as described herein. A host cell of the disclosure is alternatively referred to herein as a “cell”, a “recombinant cell” or a “recombinant host cell”. “Recombinant” in this context refers to a genetic modification having been introduced to the cell.

The host cells of the may be used in the methods described herein. For example, a method for making a compound of formula (I) and/or a compound of formula (Ia) (such as a compound of formula (V)) as described herein may comprise culturing a host cell as described herein. The term “culturing” refers to a process of multiplying living cells such that they produce an SHC enzyme as described herein. Accordingly, the associated benefits with the SHC enzymes and the methods using the SHC enzymes described herein also apply to host cells expressing the SHC enzymes and to methods using the host cells.

A nucleic acid molecule, nucleic acid construct, or vector described herein may be introduced in a host cell using standard molecular toolbox techniques available to the skilled person, which may differ depending on the host cell (e.g., a prokaryotic or a eukaryotic cell). Examples of such techniques are transfection and (viral) transduction. Additional examples of such techniques may further be found in standard handbooks such as Ausubel et al. (2003), and Sambrook and Green (2012) (supra).

The introduced (“transforming”) nucleic acid may or may not be integrated, i.e. covalently linked into a chromosome of the cell. In prokaryotes, and yeast, for example, the introduced nucleic acid may be maintained on an episomal element such as a plasmid. With respect to eukaryotic cells, a stably transfected cell is one in which the transfected nucleic acid has become integrated into a chromosome so that it is inherited by daughter cells through chromosome replication. This stability is demonstrated by the ability of the eukaryotic cell to establish cell lines or clones comprised of a population of daughter cells containing the introduced nucleic acid. In prokaryotic and/or eukaryotic cells, integration of nucleic acids into the host cell's genome may, for example occur through cellular DNA repair mechanisms such as homologous recombination, non-homologous end-joining, and the like. Integration of nucleic acids may be mediated by introduction of a break into a chromosome of a a host cell, for example using a nuclease such as a zinc-finger nuclease (ZFN), a transcription activator-like effector nuclease (TALEN), a clustered regularly interspaced shorted palindromic repeat (CRISPR)-Cas-associated nuclease, a recombinase (e.g., a Cre recombinase) and the like. Nucleases and recombinases are known to the skilled person and their utilization in transformation of host cells is further discussed in standard handbooks such as Musunuru Kiran, Genome Editing: A Practical Guide to Research and Clinical Applications, 1^{st} Edition, Academic Press (2021), and Ghosh Dipanjan (Ed), Advances in CRISPR/Cas and Related Technologies, 1^{st} Edition, Academic Press (2021), both of which are incorporated herein by reference in their entireties.

Typically, the introduced nucleic acid is not originally present in the recipient host cell, but it is within the scope of the disclosure to isolate a nucleic acid from a given host, and to subsequently introduce one or more additional copies of that nucleic acid into the same host, e.g., to enhance production of the product of a gene or alter the expression pattern of a gene, such as one expressing an SHC enzyme described herein. In some instances, the introduced nucleic acid will modify or even replace an endogenous nucleic acid sequence, e.g. by homologous recombination or site-directed mutagenesis.

Accordingly, expression of an SHC enzyme by a host cell described herein may refer to homologous expression (wherein the nucleotide sequence encoding said enzyme is originally present in the cell) or heterologous expression (wherein the nucleotide sequence encoding said enzyme is not originally present in the cell).

Suitable host cells may be selected from prokaryotic or eykaryotic cells, for example bacteria, archaea, yeasts, filamentous fungi, algae, plant cells, animal cells, amphibian cells (including melanophore cells), insect cells, worm cells, and mammalian cells.

Algae host cells may be selected from suitable groups known in the art such as Botryococcus braunii, Chlorella, Dunaliella tertiolecta, Gracilaria, Pleurochrysis carterae, and Sargassum. Yeast host cells may be selected from suitable groups known in the art such as Saccharomyces (for example, Saccharomyces cerevisiae, Saccharomyces bayanus, Saccharomyces boulardii), Candida (for example, Candida utilis, krusei), (for Schizosaccharomyces pombe, Candida Schizosaccharomyces example, Schizosaccharomyces japonicus), Pichia or Hansenula (for example, Pichia pastoris or Pichia pastoris (Komagatella phaffi) or Hansenula polymorpha), Yarrowia, Kluyveromyces, and Brettanomyces (for example, Brettanomyces claussenii).

Filamentous fungal host cells may be selected from suitable groups known in the art such as Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocaffimastix, Neurospora, Paecilomyces, Peniciffium, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria. Species include Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Penicillium chrysogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

Insect host cells and worm cells may be selected from suitable groups knowin in the art such as Sf9 cells, Sf21 cells, Spodoptora frugiperda cells, Caenorhabditis cells (such as Caenorhabditis elegans cells), and derivatives thereof. Mammalian host cells may be selected from suitable groups known in the art such as human cells, Chinese hamster ovary (CHO) cells, COS cells (including Cos-1 and Cos-7), HEK293 cells, HEK293T cells, HEK293 T-Rex™ cells, PerC6™ cells, Hela cells, Jurkat cells, hybridomas, and derivatives thereof. Plant host cells may be selected from suitable groups known in the art, such as the group of Arabidopsis, and the like.

Preferred host cells are bacterial host cells, which may be selected from suitable groups known in the art. Bacterial host cells include both Gram-negative and Gram-positive bacteria such as Bacillus (for example Bacillus cereus, Bacillus anthracis, Bacillus thuringiensis, Bacillus mycoides, Bacillus pseudomycoides, Bacillus cytotoxicus, Bacillus coagulans, Bacillus subtilis, and Bacillus licheniformis), Paenibacillus, Streptomyces, Micrococcus, Corynebacterium, Acetobacter, Cyanobacteria, Salmonella, Rhodococcus, Pseudomonas, Lactobacillus, Lactococcus, Enterococcus, Alcaligenes, Klebsiella, Paenibacillus, Arthrobacter, Corynebacterium, Brevibacterium, Thermus aquaticus, Pseudomonas stutzeri, Clostridium thermocellus, Escherichia (for example Escherichia coli), including strains thereof. Among bacterial host cells, E. coli and strains thereof are preferred. Multiple libraries of mutants, plasmids, detailed computer models of metabolism, transformation methods, and other information is available in the art for E. coli, allowing for rational design of various genetic modules to enhance product yield of recombinant host cells expressing enzymes. Preferably, an E. coli host cell is an E. coli strain which is recognized as safe by industry and regulatory authorities (including but not limited to the K12 and BL21 strains). Utilizing E. coli as a host cell may be advantageous in making a compound of formula (I) from a compound of formula (II), given that low cost and industrially economical processes may be relatively easily designed for this host cell.

Several host cells and strains belonging to the groups discussed above are readily accessible to the public in a number of well-known collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the host cell is a bacterial host cell selected from the group of Escherichia, Streptomyces, Bacillus, Pseudomonas, Lactobacillus, and Lactococcus, and strains thereof, preferably it is Escherichia coli and strains thereof. Examples of suitable host cells and transformation methods may further be found in WO2021/209482.

Culturing of a host cell described herein may be performed in a conventional manner. Suitable cell culturing methods are known to the skilled person and are discussed, for example, in van′t Riet, K. and Tramper, J., 1st edition, Basic Bioreactor Design, CRC Press, NY, 1991 (incorporated herein by reference in its entirety). Such methods include, but are not limited to, submerged fermentation in liquid media, surface fermentation on liquid media and solid-state fermentations. Cell culturing may, for example, be performed by cultivation in micro-titer plates, shake-flasks, small-scale benchtop bioreactors, medium-scale bioreactors and/or large-scale bioreactors in a laboratory and/or an industrial setting. Suitable cell culturing modes include, but are not limited to, continuous, batch and/or fed-batch culture as well as their combinations. Typically, the cells are grown to a particular density (measurable e.g., as optical density (OD)) to produce a sufficient biomass and/or SHC enzyme for a bioconversion reaction as described earlier herein to occur.

In some embodiments, there is provided a method of making a compound of formula (I) in a cellular system, comprising producing an SHC enzyme enzyme described herein under suitable conditions in a cellular system, feeding a compound of formula (II) to the cellular system, converting the compound of formula (II) to a compound of formula (I) using the SHC enzymes produced using the cellular system, collecting the compound of formula (I) from the cellular system, and optionally isolating and/or purifying the compound of formula (I).

In some embodiments, there is provided a method of making a compound of formula (Ia), preferably a compound of formula (V), in a cellular system, comprising producing an SHC enzyme enzyme described herein under suitable conditions in a cellular system, feeding a compound of formula (IIa) to the cellular system, converting the compound of formula (IIa) to a compound of formula (Ia), preferably to a compound of formula (V), using the SHC enzymes produced using the cellular system, collecting the compound of formula (Ia), preferably the compound of formula (V), from the cellular system, and optionally isolating and/or purifying the compound of formula (Ia), preferably the compound of formula (V).

In some embodiments, there is provided a method of making a mixture comprising a compound of formula (I) and a compound of formula (Ia) in a cellular system, comprising producing an SHC enzyme enzyme described herein under suitable conditions in a cellular system, feeding a mixture comprising the compound of formula (II) and the compound of formula (IIa) to the cellular system, converting the compound of formula (II) to a compound of formula (I) and the compound of formula (IIa) to a compound of formula (Ia) using the SHC enzymes produced using the cellular system, collecting the compound of formula (I) and the compound of formula (Ia) from the cellular system, and optionally isolating and/or purifying the compound of formula (I) and/or the compound of formula (Ia).

In some embodiments, there is provided a method of making a mixture comprising a compound of formula (I) and a compound of formula (V) in a cellular system, comprising producing an SHC enzyme enzyme described herein under suitable conditions in a cellular system, feeding a mixture comprising the compound of formula (II) and the compound of formula (IIa) to the cellular system, converting the compound of formula (II) to a compound of formula (I) and the compound of formula (IIa) to a compound of formula (V) using the SHC enzymes produced using the cellular system, collecting the compound of formula (I) and the compound of formula (V) from the cellular system, and optionally isolating and/or purifying the compound of formula (I) and/or the compound of formula (V).

Expression of other nucleic acids may serve to enhance the methods, for example by enhancing the activity of the cellular system used in the bioconversion reactions described above.

In some embodiments, there is provided a method of making a compound of formula (I), comprising culturing host cells comprising a nucleic acid comprising a nucleotide sequence encoding an SHC enzyme described herein, producing the SHC enzyme in the host cells, adding a compound of formula (II) to the cell culture, incubating the cell culture under conditions of pH, temperature, and optionally a solubilizing agent (such as SDS), suitable to promote the conversion of the compound of formula (II) to a compound of formula (I), collecting the compound of formula (I), and optionally isolating and/or purifying the compound of formula (I).

In some embodiments, there is provided a method of making a compound of formula (Ia), preferably a compound of formula (V), comprising culturing host cells comprising a nucleic acid comprising a nucleotide sequence encoding an SHC enzyme described herein, producing the SHC enzyme in the host cells, adding a compound of formula (IIa) to the cell culture, incubating the cell culture under conditions of pH, temperature, and optionally a solubilizing agent (such as SDS), suitable to promote the conversion of the compound of formula (IIa) to a compound of formula (Ia), preferably to a compound of formula (V), collecting the compound of formula (Ia), preferably the compound of formula (V), and optionally isolating and/or purifying the compound of formula (I), preferably the compound of formula (V).

In some embodiments, there is provided a method of making a mixture comprising a compound of formula (I) and a compound of formula (Ia), comprising culturing host cells comprising a nucleic acid comprising a nucleotide sequence encoding an SHC enzyme described herein, producing the SHC enzyme in the host cells, adding a mixture comprising a compound of formula (II) and a compound of formula (IIa) to the cell culture, incubating the cell culture under conditions of pH, temperature, and optionally a solubilizing agent (such as SDS), suitable to promote the conversion of the compound of formula (II) to a compound of formula (I) and the conversion of the compound of formula (IIa) to a compound of formula (Ia), collecting the compound of formula (I) and the compound of formula (Ia), and optionally isolating and/or purifying the compound of formula (I) and/or the compound of formula (Ia).

In some embodiments, there is provided a method of making a mixture comprising a compound of formula (I) and a compound of formula (V), comprising culturing host cells comprising a nucleic acid comprising a nucleotide sequence encoding an SHC enzyme described herein, producing the SHC enzyme in the host cells, adding a mixture comprising a compound of formula (II) and a compound of formula (IIa) to the cell culture, incubating the cell culture under conditions of pH, temperature, and optionally a solubilizing agent (such as SDS), suitable to promote the conversion of the compound of formula (II) to a compound of formula (I) and the conversion of the compound of formula (IIa) to a compound of formula (V), collecting the compound of formula (I) and the compound of formula (V), and optionally isolating and/or purifying the compound of formula (I) and/or the compound of formula (V).

The bioconversion reactions may be enhanced by adding more biocatalyst, and optionally a solubilizing agent such SDS to the cell cultures described above.

Cell culture conditions suitable for growth and enzyme production by host cells may vary depending on the host cells. Such conditions are known to the skilled person, and are further, for example, typically provided by cell culture collections from which the host cells may be obtained. Cell culture conditions and bioconversion reaction conditions may be the same or may differ. The skilled person further understands that a cell may initially be cultured under conditions that are optimal for cellular growth and/or enzyme production, and the conditions may subsequently be adjusted to conditions that are optimal for the bioconversion reaction to take place, which may be the same or different.

The term “biocatalyst” as used herein may refer to an SHC enzyme as described herein itself, but also to a host cell expressing said enzyme, a membrane fraction of said host cell, a cell lysate, cellular debris, or a cell-free extract, the common feature being that the SHC enzymatic activity is present.

In some embodiments, the biocatalyst is a recombinant host cell producing an SHC enzyme, which may optionally be in suspension or an immobilized format.

In some embodiments, the biocatalyst is a membrane fraction or a liquid fraction prepared from a recombinant host cell producing an SHC enzyme using routine methods (as disclosed for example in Seitz (2012), Characterization of the substrate specificity of squalene-hopene cyclases (SHCs), PhD thesis, University of Stuttgart, available at http://dx.doi.org/10.18419/opus-1383, incorporated herein by reference in its entirety), such as a crude extract or a cell-free extract.

A biocatalyst includes whole cells collected from a cell culture (e.g., from a bioreactor cell culture), as well as cells that are still in culture (which are then used in a one-pot method, described later herein). A biocatalyst includes intact recombinant host cells and/or cell debris thereof.

A biocatalyst may be immobilized. Immobilization of host cells and/or SHC enzymes may be achieved by any means known to the skill person, e.g., as discussed in Seitz et al. (supra), and in standard handbooks such as Guisan, J. M., Bolivar, J. M., López-Gallego, F., Rocha-Martin, J. (Eds.), Immobilization of Enzymes and Cells: Methods and Protocols, Springer US, USA, 2020 (incorporated herein by reference in its entirety). An example of an immobilization method involves polymerizing or solidifying a spore- or cell-containing solution. Examples of polymerizable or solidifyable solutions include alginate, A-carrageenan, chitosan, polyacrylamide, polyacrylamide-hydrazide, agarose, polypropylene, polyethylene glycol, dimethyl acrylate, polystyrene divinyle benzene, polyvinyl benzene, polyvinyl alcohol, epoxy carrier, cellulose, cellulose acetate, photocrosslinkable resin, prepolymers, urethane, and gelatin. Another example of an immobilization method involves cell adsorption onto a support. Examples of such supports include bone char, cork, clay, resin, sand porous alumina beads, porous brick, porous silica, celite, or wood chips. The host cells can colonize the support and form a biofilm. Another example of an immobilization method involves the covalent coupling of the host cells to a support using chemical agents like glutaraldehyde, o-dianisidine, polymeric isocyanates, silanes (e.g., as discussed in U.S. Pat. Nos. 3,983,000; 4,071,409; 3,519,538 and 3,652,761, all of which are incorporated herein by reference in their entireties), hydroxyethyl acrylate, transition metal-activated supports, cyanuric chloride, sodium periodate, toluene, and the like. Cultured host cells can be immobilized in any phase of their growth, for example after a desired cell density in the culture has been reached.

In some embodiments, the host cells are cultured, harvested, washed, and optionally stored (e.g., frozen or lyophilized)) before their use in the bioconversion reaction.

In some embodiments, the host cells are cultured and the culture conditions are then adjusted without harvesting and washing of the cells prior to the bioconversion reaction to be suitable for the reaction to occur. This one-step (or “one-pot”) method may be advantageous as it may simplify the process. The culture medium used to grow the cells in these embodiments may also be used as the reaction mixture in the bioconversion reaction. A compound of formula (II), a compound of formula (IIa), and/or a mixture comprising a compound of formula (II) and a compound of formula (IIa) may be present in the culture from the beginning or may be added subsequently to the culture phase of the method.

Cell culturing can take place using a culture medium (alternatively referred to herein as growth medium) comprising suitable nutrients, such as carbon and nitrogen sources, and optionally additional compounds such as inorganic salts and vitamins. Suitable culture media may vary depending on the host cell, and are available from commercial suppliers or may be prepared using published compositions (e.g. in catalogues of the Centraalbureau Voor Schimmelcultures collection (CBS) which are generally available for each host cell). Suitable carbon sources include any molecule that can be metabolized by a recombinant host cell to facilitate growth and/or production of an SHC enzyme as described herein for the conversion of a compound of formula (II) to a compound of formula (I) and/or the conversion of a compound of formula (IIa) to a compound of formula (Ia) (such as a compound of formula (V)). Examples of suitable carbon sources include, but are not limited to, sucrose (e.g., pure or as found in mixtures such as molasses), fructose, xylose, glycerol, glucose, ethanol, cellulose, starch, cellobiose or any other carbohydrate containing polymer, as well as mixtures thereof. Examples of suitable nitrogen sources include, but are not limited to, urea, ammonia, ammonium salts, nitrate salts, as well as mixtures thereof. Complex carbon and nitrogen sources, such as a protein hydrolysate, tryptone, soybean meal, corn steep liquor, whey protein hydrolysate, egg protein hydrolysate, casein hydrolysate, yeast-extract, and the like, are also suitable.

In embodiments wherein the host cell is a yeast cell, a preferred carbon source may be selected from sucrose, fructose, xylose, ethanol, glycerol, glucose, as well as mixtures thereof.

A host cell may be cultured in a rich medium (e.g., LB-medium, Bacto-tryptone yeast extract medium, and the like), or a defined medium, for example a defined minimal medium.

In some embodiments, a defined minimal medium such as an M9A medium or another defined minimal medium is used for cell culturing. An M9A medium may comprise: 14 g/L KH₂PO₄, 16 g/L K₂HPO₄, 1 g/L Na₃Citrate.2H₂O, 7.5 g/L (NH₄)₂SO₄, 0.25 g/L MgSO₄·7H₂O, 0.015 g/L CaCl₂)·2H₂O, 5 g/L glucose and 1.25 g/L yeast extract.

In some embodiments, a rich medium such as an LB-medium or another rich medium is used for cell culturing. An LB medium may comprise: 10 g/L tryptone, 5 g/L yeast extract, and 5 g/L NaCl. Additional examples of mineral media and M9 mineral media may be, for example found in U.S. Pat. No. 6,524,831B2 and US2003/0092143A1.

An additional example of a suitable minimal medium may be prepared as follows:

For 350 ml of culture: 307 ml of H₂O may be added to 35 ml of citric acid/phosphate stock solution (containing 133 g/L KH₂PO₄, 40 g/L (NH₄)₂HPO₄, 17 g/L citric acid.H₂O, and having a pH of 6.3) and the pH may be adjusted to 6.8 with 32% w/v NaOH. The solution may be autoclaved under routine conditions used in the art and post-autoclaving 0.85 ml 50% w/v MgSO₄·7H₂O stock solution (see below), 0.035 ml trace elements stock solution (see below), 0.035 ml thiamin stock solution (see below), and 7 ml of 20% w/v glucose solution may be added.

The trace elements stock solution may comprise: 50 g/L Na₂EDTA.2H₂O, 20 g/L FeSO₄·7H₂O, 3 g/L H₃BO₃, 0.9 g/L MnSO₄·2H₂O, 1.1 g/L CoCl₂, 80 g/L CuCl₂, 240 g/L NiSO₄·7H₂O, 100 g/L KI, 1.4 g/L (NH₄)₆MO₇O₂₄·4H₂O, 1 g/L ZnSO₄·7H₂O, in deionized water. The thiamin stock solution may comprise: 2.25 g/L thiamin. HCl in deionized water. The MgSO₄ stock solution may comprise: 50% w/v MgSO₄·7H₂O in deionized water.

Typically, an optimum pH for growing cells in a cell culture is from 4 to 8. An optimum pH for the bioconversion reaction may differ depending on the properties of the SHC enzyme used. The pH of the bionversion reaction mixture may be from 4 to 8, preferably from 5 to 6.5, more preferably from 5.5 to 6.1. Adjustment and regulation of the pH in a cell culture or reaction mixture may be done by any suitable technique known by the skilled person, for example by addition of stock solutions of acids and bases, or addition of buffers. Non-limiting examples of buffers include a citric acid buffer and a succinic acid buffer.

Typically, an optimum temperature for cell culture and/or the bioconversion reaction is from 15° C. to 60° C., preferably from 25° C. to 50° C., more preferably from 25° C. to 45° C. An optimum pH for the bioconversion reaction may differ depending on the properties of the SHC enzyme used. In some embodiments, an optimum temperature is 30° C. The temperature may be kept constant throughout the cell culture and/or bioconversion reaction, or may be altered.

Specific optimal pH and temperature conditions for specific preferred enzymes described herein are given in Table 5.

Typically, cell culturing is performed under anaerobic, aerobic, or oxygen-limited conditions. The requirement for oxygen will vary depending on the host cell and culture mode, and will be known to the skilled person. Aerobic conditions are conditions in which the oxygen consumption of the host cell is not limited by oxygen availability. Under oxygen-limited conditions, oxygen consumption is limited by oxygen availability. Oxygen may be supplied to a culture by any known method, e.g., by shaking under an air atmosphere, by stirring, by sparging air and/or oxygen in the culture, and others.

Optionally, a solubilizing agent such as a surfactant, a detergent, a solubility enhancer, a water miscible organic solvent, and the like, may be added to the cell culture or to the bioconversion reaction mixture. As used herein, the term “surfactant” refers to a component that lowers the surface tension (or interfacial tension) between two liquids or between a liquid and a solid. Surfactants may act as detergents, wetting agents, emulsifiers, foaming agents, and dispersants. Examples of surfactants include, but are not limited to, Triton X-100, Tween 80, taurodeoxycholate, sodium taurodeoxycholate, sodium dodecyl sulfate (SDS), and/or sodium lauryl sulfate (SLS).

Whilst Triton X-100 may be used to partially purify an SHC enzyme (in soluble or membrane fraction/suspension form), it may also be used in the bioconversion reaction (see for example the disclosure in Seitz (2012, supra) as well as the disclosures of Neumann and Simon (1986), Biol Chem 367:723-729, and JP2009060799, both of which are incorporated herein by reference in their entireties.

A preferred solubilizing agent is SDS. Without wishing to be bound by theory, the use of SDS with recombinant host cells may be advantageous as the SDS may interact advantageously with the host cell membrane in order to make the SHC enzyme (which is a membrane bound enzyme) more accessible to a compound of formula (II) and/or a compound of formula (IIa) substrate. In addition, the inclusion of SDS at a suitable level in the cell culture and/or bioconversion reaction mixture may improve the properties of the emulsion (e.g., of compound of formula (II) and/or compound of formula (IIa) in water) and/or improve the access of the compound of formula (II) and/or compound of formula (IIa) substrate to the SHC enzyme within the host.

The skilled person understands that the optimal concentration of the solubilising agent (e.g., SDS) used in the bioconversion reactions described herein may vary depending on the cell biomass amount and the substrate concentration. An optimum concentration of the solubilising agent (e.g., SDS) for the bioconversion reaction may also differ depending on the properties of the SHC enzyme used. Determination of an appropriate concentration can be made by routine experimentation. In the methods of the disclosure, the SDS/cells concentration ratio may preferably be from 10:1 to 20:1, more preferably from 15:1 to 18:1, when the ratio of biocatalyst to a compound of formula (II) and/or a compound of formula (IIa) is 2:1 or about 2:1. In some embodiments, the SDS/cells concentration ratio may preferably be 10:1 or about 10:1, 11:1 or about 11:1, 12:1 or about 12:1, 13:1 or about 13:1, 14:1 or about 14:1, 15:1 or about 15:1, 16:1 or about 16:1, 17:1 or about 17:1, 18:1 or about 18:1, 19:1 or about 19:1, or 20:1 or about 20:1, when the ratio of biocatalyst to a compound of formula (II) and/or a compound of formula (IIa) is 2:1 or about 2:1.

In the methods of the disclosure, the SDS concentration may, for example, be from 0.001% to 0.03%, preferably from 0.01% to 0.025%, more preferably 0.01%-0.02% (w/v %). These ranges correspond to ranges used in a reaction containing cells at an OD of 10 or about 10 (measured at 650 nm). The skilled person understands that suitable SDS concentrations are not limited to these ranges and may be increased or decreased when the cell concentration is respectively increased or decreased, in order to maintain a constant SDS/cells concentration ratio.

Specific exemplary SDS concentrations for specific preferred enzymes described herein are given in Table 5. Additional exemplary SDS concentrations for bioconversion reactions utilizing host cells as described herein are given in Examples 8 and 9.

In embodiments wherein a compound of formula (II), a compound of formula (IIa), or a mixture comprising a compound of formula (II) and a compound of formula (IIa), is added to a cell culture or reaction mixture, its addition (“feeding”), may be done using any standard means available to the skilled person (e.g., through tubing using a peristaltic pump, using an infusion syringe, and the like).

A compound of formula (II) and/or compound of formula (IIa) may be oil soluble and provided dissolved in oil. In cases wherein a biocatalyst as described earlier herein is present in an aqueous phase, addition of a compound of formula (II) and/or a compound of formula (IIa) will result in a three phase system (comprising an aqueous phase, a solid phase, and an oil phase). This may be the case even when SDS is present in the cell culture and/or reaction mixture.

In some embodiments, a cell culture is a continuous culture. Such a culture may be advantageous in some cases as it could result in improved production of a compound of formula (I) and/or of a compound of formula (Ia) (such as a compound of formula (V)).

In some embodiments, the bioconversion of a compound of formula (II) to a compound of formula (I) in the presence of a host cell expressing an SHC enzyme as described herein results in conversion of a compound of formula (II) to a compound of formula (I) of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, given in mol percent and based on the mols of compound of formula (II) employed. Preferably, the yield is from 5 to 100, from 10 to 100, from 20 to 100, from 30 to 100, from 35 to 100, more preferably from 40 to 100, from 45 to 100, from 50 to 100, from 60 to 100, or from 70 to 100 mol percent.

In some embodiments, the bioconversion of a compound of formula (IIa) to a compound of formula (Ia), preferably into a compound of formula (V), in the presence of a host cell expressing an SHC enzyme as described herein results in conversion of a compound of formula (IIa) to a compound of formula (Ia), preferably to a compound of formula (V), of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, given in mol percent and based on the mols of compound of formula (IIa) employed. Preferably, the yield is from 5 to 100, from 10 to 100, from 20 to 100, from 30 to 100, from 35 to 100, more preferably from 40 to 100, from 45 to 100, from 50 to 100, from 60 to 100, or from 70 to 100 mol percent.

In some embodiments, the bioconversion of a compound of formula (II) to a compound of formula (I) and/or the bioconversion of a compound of formula (IIa) to a compound of formula (Ia), in a mixture comprising a compound of formula (II) and a compound of formula (IIa), in the presence of a host cell expressing an SHC enzyme as described herein results in conversion of a compound of formula (II) to a compound of formula (I) and/or of a compound of formula (IIa) to a compound of formula (Ia), of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, given in mol percent and based on the mols of compound of formula (II) and compound of formula (IIa) employed. Preferably, the yield of compound (I) is from 5 to 100, from 10 to 100, from 20 to 100, from 30 to 100, from 35 to 100, more preferably from to 100, from 45 to 100, from 50 to 100, from 60 to 100, or from 70 to 100 mol percent. Preferably, the yield of compound (la) is from 5 to 100, from 10 to 100, from 20 to 100, from 30 to 100, from 35 to 100, more preferably from 40 to 100, from 45 to 100, from 50 to 100, from 60 to 100, or from 70 to 100 mol percent.

In some embodiments, the bioconversion of a compound of formula (II) to a compound of formula (I) and/or the bioconversion of a compound of formula (IIa) to a compound of formula (V), in a mixture comprising a compound of formula (II) and a compound of formula (IIa), in the presence of a host cell expressing an SHC enzyme as described herein results in conversion of a compound of formula (II) to a compound of formula (I) and/or of a compound of formula (IIa) to a compound of formula (V), of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, given in mol percent and based on the mols of compound of formula (II) and compound of formula (IIa) employed. Preferably, the yield of compound (I) is from 5 to 100, from 10 to 100, from 20 to 100, from 30 to 100, from 35 to 100, more preferably from to 100, from 45 to 100, from 50 to 100, from 60 to 100, or from 70 to 100 mol percent. Preferably, the yield of compound (V) is from 5 to 100, from 10 to 100, from 20 to 100, from 30 to 100, from 35 to 100, more preferably from 40 to 100, from 45 to 100, from 50 to 100, from 60 to 100, or from 70 to 100 mol percent.

In some embodiments, a preferred rate of a compound of formula (II) and/or compound of formula (IIa) conversion and/or obtained conversion of a compound of formula (II) to a compound of formula (I) and/or of a compound of formula (IIa) to a compound of formula (Ia) (such as a compound of formula (V)) are determined over a defined time period of for example, 4, 6, 8, 10, 12, 16, 20, 24, 36, 48, 72, 96, 120, 142, 144, 150, or 168 hours, preferably of 24 hours, during which a compound of formula (II) is converted into a compound of formula (I) and/or a compound of formula (IIa) is converted into a compound of formula (Ia) (such as a compound of formula (V)) by a recombinant host cell comprising a nucleotide sequence encoding an SHC enzyme as described herein, and which has produced the SHC enzyme.

In some embodiments, the bioconversion reaction is carried out under a temperature value of, for example, 25° C., 30° C., 35° C., 40° C., 50° C. or 60° C. In some embodiments, the obtained conversion of a compound of formula (II) to a compound of formula (I) and/or of a compound of formula (IIa) to a compound of formula (Ia) (such as a compound of formula (V)) and/or the rate of a compound of formula (II) and/or a compound of formula (IIa) conversion are determined by carrying out the reaction at a temperature range from 25° C. to 55° C., preferably from 30° C. to 40° C., over a period of 24-72 hours. In some embodiments, the time period is extended, for example up to a total of 150 hours or longer.

In some embodiments, a recombinant host cell comprising a nucleotide sequence encoding an SHC enzyme described herein shows an at least 5%, 10%, 20%, 30%, 40%, 50%, 60%, 70%, 80%, 90%, 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold higher conversion of a compound of formula (II) to a compound of formula (I) and/or of a compound of formula (IIa) to a compound of formula (Ia) (such as a compound of formula (V)) and/or rate of a compound of formula (II) and/or a compound of formula (IIa) conversion compared to a recombinant host cell expressing a nucleotide sequence encoding the parental SHC enzyme under the same conditions, preferably under conditions that have been individually defined as being optimal for the activity of the SHC enzyme considered.

In some embodiments, a method as described herein is performed at a host cell and/or a compound of formula (II) and/or a compound of formula (IIa) concentration (in a liquid culture) of 5 g/L or higher, 10 g/L or higher, 20 g/L or higher, 30 g/L or higher, 40 g/L or higher, 50 g/L or higher, 60 g/L or higher, 70 g/L or higher, 80 g/L or higher, 90 g/L or higher, 100 g/L or higher, 110 g/L or higher, 120 g/L or higher, 130 g/L or higher, 135 g/L or higher, 150 g/L or higher, 175 g/L or higher, or 200 g/L or higher, or 250 g/L or higher.

In some embodiments, a method as described herein is performed at a weight ratio of a host cell to the substrate of 0.1-4 to 1 or of about 0.1-4 to 1 (0.1-4:1), 0.1-3 to 1 or of about 0.1-3 to 1 (0.1-3:1), 0.1-2 to 1 or of about 0.1-2 to 1 (0.1-2:1), of 0.25-2 to 1 or of about 0.25-2 to 1 (0.25-2:1), of 0.5-2 to 1 or of about 0.5-2 to 1 (0.5-2:1), of 0.1 to 1 or of about 0.1 to 1 (0.1:1), of 0.5 to 1 or of about 0.5 to 1 (0.5:1), of 1 to 1 or of about 1 to 1 (1:1), of 1.5 to 1 or of about 1.5 to 1 (1.5:1), or of 2 to 1 or of about 2 to 1 (2:1), preferably of 0.1 to 1 or of about 0.1 to 1 (0.1:1), of 0.5 to 1 or of about 0.5 to 1 (0.5:1), or of 1 to 1 or of about 1 to 1 (1:1).

An SHC enzyme described herein may exhibit improved reaction performance as compared to its parent enzyme at these concentrations, as described earlier herein. Reaction performance of an SHC enzyme described herein may be assessed using any of the parameters discussed earlier herein, such as productivity, total conversion or increased rate of substrate conversion, or yield of a compound of formula (I) and/or a compound of formula (Ia) (such as a compound of formula (V)), which may be improved by at least 5%, 10%, 20%, 30%, 40%, 50%, 60%, 70%, 80%, 90%, 100% (2-fold), 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold, 200-fold, 500-fold, or 1000-fold as compared to the reaction performance of its parent SHC enzyme.

TABLE 1
Sequences

| SEQ | | |
|---|---|---|
| ID NO | Name | Sequence |
| 2 | wt | ATGATCATTCTGCTCAAGGAAGTCCAGCTGGAGATTCAGCGCCGCATCGCCTATCTGCGTCCAA |
| | BmeSHC | CCCAGAAGAATGACGGTTCGTTCCGCTACTGCTTTGAGACAGGTGTTATGCCCGATGCCTTCCT |
| | DNA | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 1 | wt | MIILLKEVQLEIQRRIAYLRPTQKNDGSFRYCFETGVMPDAFLIMLLRTFDLDKEVLIKQLTER |
| | BmeSHC | IVSLQNEDGLWTLFDDEEHNLSATIQAYTALLYSGYYQKNDRILRKAERYIIDSGGISRAHELT |
| | protein | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 3 | 3G6 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTCCACAACCCAAATACCACACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCTCACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 4 | 3G6 | MNILLKEVQLEIQRRIAYLRPTQKNDGSFRYCFEAGVMPDAFLIMLLRTFDLDKEVLIKQLTER |
| | protein | IVSLQNEDGLWTLFDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTTPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 5 | 59B7 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCTTTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCGTCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 6 | 59B7 | MIILLKEVQLEIQRRIAYLRPTQKNDGSFRYCFETGVMPDAFLIMLLRTFDLDKEVLIKQLTER |
| | protein | IVSLQNEDGLWTLFDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 7 | 13E9 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 8 | 13E9 | MIILLKEVQLEIQRRIAYLRPTQKNDGSFRYCFETGVMPDAFLIMLLRTFDLDKEVLIKQLTER |
| | protein | IVSLQNEDGLWTLFDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 9 | 50D3 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCACCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGACGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCcACGTGCA |
| | | CGTCGAAAACTCGACATCAACGGTGTGGGATACCGCATTGCTGTCCTATGCAATGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGCGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 10 | 50D3 | MIILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYITDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQVRRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYAMQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTAQQTHYPTGIGLPGQFYIQYHSYNDIFPLLALAHYAKKHSS |
| 11 | 73F9 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCGTCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 12 | 73F9 | MIILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGVNWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 13 | 83D1 | ATGATCATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCCGGGAACAAGAAATTCAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGTATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 14 | 83D1 | MIILPKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 15 | 114E1 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACTTCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTAACACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 16 | 114E1 | MIILLKEVQLEIQRRIAYLRPTQKNDGSFRYCFETGVMPDAFLIMLLRTEDLDKEVLIKQLTER |
| | protein | IVSLQNEDGLWTLFDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKFSLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALTRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 17 | #15 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 18 | #15 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 19 | #21 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 20 | #21 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 21 | #42 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 22 | #42 | MNILPKEVQLEIQRRIAYLRPTQKNDGSFRYCFEAGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 23 | #47 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 24 | #47 | MNILPKEVQLEIQRRIAYLRPTQKNDGSFRYCFEAGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKFSLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 25 | #56 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 26 | #56 | MNILPKEVQLEIQRRIAYLRPTQKNDGSFRYCFEAGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 27 | #96 | ATGATCATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGACGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 28 | #96 | MIILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKFSLTSRHTPS |
| | | LSHLDVREQKQESEETTQVRRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 29 | #179 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 30 | #179 | MNILPKEVQLEIQRRIAYLRPTQKNDGSFRYCFEAGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 31 | #180 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTACGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 32 | #180 | MNILPKEVQLEIQRRIAYLRPTQKNDGSFRYCFEAGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKFSLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWYGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 33 | #182 | ATGAACATTCTGCCCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGGCAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCACTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGACGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 34 | #182 | MNILPKEVQLEIQRRIAYLRPTQKNDGSFRYCFEAGVMPDAFLIMLLRTFDLDKEVLIKQLTER |
| | protein | IVSLQNEDGLWTLFDDEEHNLSATIQAYTALLYSGYYQKNDRILRKAERYIIDSGGISRAHELT |
| | | RWMLSVNGLYEWPKLFYLPLSLLLVPTYVPLNFYELSTYARIHFVPMMVAGNKKESLTSRHTPS |
| | | LSHLDVREQKQESEETTQVRRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 35 | #188 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 36 | #188 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 37 | #189 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGAAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 38 | #189 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQESRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 39 | #192 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACTCAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 40 | #192 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 41 | #193 | ATGAACATTCTGCTCAAGGAAGTCCAGCTGGAGATTCAGCGCCGCATCGCCTATCTGCGTCCAA |
| | DNA | CCCAGAAGAATGACGGTTCGTTCCGCTACTGCTTTGAGACAGGTGTTATGCCCGATGCCTTCCT |
| | | GATCATGCTTCTGCGCACCTTCGATTTAGATAAAGAGGTTTTAATTAAGCAGCTTACGGAACGT |
| | | ATTGTGAGCCTTCAAAATGAAGACGGCCTGTGGACCCTCTTCGACGACGAGGAGCATAACCTCA |
| | | GCGCAACAATTCAGGCCTACACCGCCCTCTTATACAGCGGCTATTATCAAAAGAATGACCGTAT |
| | | CCTTCGTAAAGCCGAGCGCTACATCATCGACTCTGGCGGTATTTCCCGCGCGCATTTCCTGACA |
| | | CGCTGGATGCTGAGTGTCAATGGTTTATATGAATGGCCTAAACTTTTCTATCTCCCTCTGAGCT |
| | | TGCTGTTGGTGCCAACCTACGTTCCATTAAATTTCTACGAACTCTCCGCTTATGCTCGCATCCA |
| | | TTTCGTACCAATGATGGTTGCTGGGAACAAGAAATTTAGTCTTACCAGCCGCCACACCCCGAGC |
| | | TTATCACACCTTGATGTGCGCGAGCAGAAACAAGAAAGCGAAGAGACGACCCAAGTAAGTCGTG |
| | | CGAGTATCTTTCTTGTTGACCACCTCAAGCAACTTGCATCGTTGCCTAGTTATATCCATAAGTT |
| | | GGGCTACCAAGCCGCAGAGCGTTACATGCTTGAGCGTATCGAGAAAGATGGGACACTGTACAGC |
| | | TACGCAACGTCCACCTTCTTCATGATCTACGGCCTTCTGGCCCTGGGCTACAAGAAAGACTCGT |
| | | TCGTAATCCAAAAGGCAATTGATGGCATCTGTTCACTTCTTTCAACCTGTTCGGGCCACGTGCA |
| | | CGTCGAAAACTCGACATCAACGGTGTGGGATACCGCATTGCTGTCCTATGCATTGCAGGAAGCC |
| | | GGTGTCCCACAACAAGATCCTATGATCAAAGGAACTACCCGCTATCTGAAGAAGCGCCAACACA |
| | | CTAAGCTTGGGGACTGGCAATTTCACAACCCAAATACCGCACCCGGCGGTTGGGGTTTCTCTGA |
| | | CATTAATACAAATAATCCAGACCTGGATGACACTTCTGCGGCCATCCGTGCGTTATCACGTCGC |
| | | GCCCAAACAGACACGGACTACCTGGAGTCCTGGCAACGTGGCATCAATTGGCTTCTGTCGATGC |
| | | AAAATAAGGACGGTGGCTTCGCGGCATTTGAGAAGAACACGGACAGCATTTTGTTCACGTACCT |
| | | TCCACTGGAAAACGCGAAGGACGCCGCGACCGACCCTGCGACGGCCGACCTGACCGGGCGTGTG |
| | | TTGGAGTGCTTAGGTAACTTCGCCGGAATGAACAAATCACACCCTTCTATCAAGGCCGCCGTAA |
| | | AGTGGCTGTTCGATCACCAGTTGGATAACGGAAGTTGGTGCGGCCGTTGGGGCGTTTGCTACAT |
| | | CTACGGGACCTGGGCCGCGATCACAGGTTTGCGCGCAGTTGGGGTGAGCGCATCGGACCCACGT |
| | | ATTATCAAGGCGATTAATTGGCTTAAGAGTATCCAACAGGAAGACGGTGGTTTCGGCGAGTCTT |
| | | GTTATTCAGCGTCACACAAGAAGTACGTTCCTTTGTCATTCAGCACCCCGAGTCAAACGGCCTG |
| | | GGCTCTGGACGCCTTAATGACGATCTGTCCGTTAAAGGACCAAAGCGTGGAGAAGGGAATCAAG |
| | | TTCTTGCTGAATCCGAATTTGACAGAGCAACAAACACATTACCCTACCGGCATTGGCTTGCCGG |
| | | GCCAATTTTACATTCAGTACCATAGCTACAATGATATTTTCCCGTTACTGGCTCTGGCACATTA |
| | | CGCGAAGAAGCATAGCAGCTGA |
| 42 | #193 | MNILLKEVQLEIQRRIAYLRPTQKNDGSFRYCFETGVMPDAFLIMLLRTFDLDKEVLIKQLTER |
| | protein | IVSLQNEDGLWTLEDDEEHNLSATIQAYTALLYSGYYQKNDRILRKAERYIIDSGGISRAHELT |
| | | RWMLSVNGLYEWPKLFYLPLSLLLVPTYVPLNFYELSAYARIHFVPMMVAGNKKESLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASHKKYVPLSFSTPSQTAWALDALMTICPLKDQSVEKGIK |
| | | FLLNPNLTEQQTHYPTGIGLPGQFYIQYHSYNDIFPLLALAHYAKKHSS |
| 43 | wt | MAEQLVEAPAYARTLDRAVEYLLSCQKDEGYWWGPLLSNVTMEAEYVLLCHILDRVDRDRMEKI |
| | AacSHC | RRYLLHEQREDGTWALYPGGPPDLDTTIEAYVALKYIGMSRDEEPMQKALRFIQSQGGIESSRV |
| | protein | FTRMWLALVGEYPWEKVPMVPPEIMFLGKRMPLNIYEFGSWARATVVALSIVMSRQPVFPLPER |
| | | ARVPELYETDVPPRRRGAKGGGGWIFDALDRALHGYQKLSVHPFRRAAEIRALDWLLERQAGDG |
| | | SWGGIQPPWFYALIALKILDMTQHPAFIKGWEGLELYGVELDYGGWMFQASISPVWDTGLAVLA |
| | | LRAAGLPADHDRLVKAGEWLLDRQITVPGDWAVKRPNLKPGGFAFQFDNVYYPDVDDTAVVVWA |
| | | LNTLRLPDERRRRDAMTKGFRWIVGMQSSNGGWGAYDVDNTSDLPNHIPFCDFGEVTDPPSEDV |
| | | TAHVLECFGSFGYDDAWKVIRRAVEYLKREQKPDGSWFGRWGVNYLYGTGAVVSALKAVGIDTR |
| | | EPYIQKALDWVEQHQNPDGGWGEDCRSYEDPAYAGKGASTPSQTAWALMALIAGGRAESEAARR |
| | | GVQYLVETQRPDGGWDEPYYTGTGFPGDFYLGYTMYRHVFPTLALGRYKQAIERR |
| 44 | wt | MGIDRMNSLSRLLMKKIFGAEKTSYKPASDTIIGTDTLKRPNRRPEPTAKVDKTIFKTMGNSLN |
| | ZmoSHC1 | NTLVSACDWLIGQQKPDGHWVGAVESNASMEAEWCLALWFLGLEDHPLRPRLGNALLEMQREDG |
| | protein | SWGVYFGAGNGDINATVEAYAALRSLGYSADNPVLKKAAAWIAEKGGLKNIRVFTRYWLALIGE |
| | | WPWEKTPNLPPEIIWFPDNFVESIYNFAQWARATMVPIAILSARRPSRPLRPQDRLDELFPEGR |
| | | ARFDYELPKKEGIDLWSQFFRTTDRGLHWVQSNLLKRNSLREAAIRHVLEWIIRHQDADGGWGG |
| | | IQPPWVYGLMALHGEGYQLYHPVMAKALSALDDPGWRHDRGESSWIQATNSPVWDTMLALMALK |
| | | DAKAEDRFTPEMDKAADWLLARQVKVKGDWSIKLPDVEPGGWAFEYANDRYPDTDDTAVALIAL |
| | | SSYRDKEEWQKKGVEDAITRGVNWLIAMQSECGGWGAFDKDNNRSILSKIPFCDFGESIDPPSV |
| | | DVTAHVLEAFGTLGLSRDMPVIQKAIDYVRSEQEAEGAWFGRWGVNYIYGTGAVLPALAAIGED |
| | | MTQPYITKACDWLVAHQQEDGGWGESCSSYME |
| 45 | wt | MTVSTSSAFHHSPLSDDVEPIIQKATRALLEKQQQDGHWVFELEADATIPAEYILLKHYLGEPE |
| | ZmoSHC2 | DLEIEAKIGRYLRRIQGEHGGWSLFYGGDLDLSATVKAYFALKMIGDSPDAPHMLRARNEILAR |
| | protein | GGAMRANVFTRIQLALFGAMSWEHVPQMPVELMLMPEWFPVHINKMAYWARTVLVPLLVLQALK |
| | | PVARNRRGILVDELFVPDVLPTLQESGDPIWRRFFSALDKVLHKVEPYWPKNMRAKAIHSCVHF |
| | | VTERLNGEDGLGAIYPAIANSVMMYDALGYPENHPERAIARRAVEKLMVLDGTEDQGDKEVYCQ |
| | | PCLSPIWDTALVAHAMLEVGGDEAEKSAISALSWLKPQQILDVKGDWAWRRPDLRPGGWAFQYR |
| | | NDYYPDVDDTAVVTMAMDRAAKLSDLHDDFEESKARAMEWTIGMQSDNGGWGAFDANNSYTYLN |
| | | NIPFADHGALLDPPTVDVSARCVSMMAQAGISITDPKMKAAVDYLLKEQEEDGSWFGRWGVNYI |
| | | YGTWSALCALNVAALPHDHLAVQKAVAWLKTIQNEDGGWGENCDSYALDYSGYEPMDSTASQTA |
| | | WALLGLMAVGEANSEAVTKGINWLAQNQDEEGLWKEDYYSGGGFPRVFYLRYHGYSKYFPLWAL |
| | | ARYRNLKKANQPIVHYGM |
| 46 | wt | MTVTSSASARATRDPGNYQTALQSTVRAAADWLIANQKPDGHWVGRAESNACMEAQWCLALWEM |
| | BjaSHC | GLEDHPLRKRLGQSLLDSQRPDGAWQVYFGAPNGDINATVEAYAALRSLGERDDEPAVRRAREW |
| | protein | IEAKGGLRNIRVFTRYWLALIGEWPWEKTPNIPPEVIWFPLWFPFSIYNFAQWARATLMPIAVL |
| | | SARRPSRPLPPENRLDALFPHGRKAFDYELPVKAGAGGWDRFFRGADKVLHKLQNLGNRLNLGL |
| | | FRPAATSRVLEWMIRHQDFDGAWGGIQPPWIYGLMALYAEGYPLNHPVLAKGLDALNDPGWRVD |
| | | VGDATYIQATNSPVWDTILTLLAFDDAGVLGDYPEAVDKAVDWVLQRQVRVPGDWSMKLPHVKP |
| | | GGWAFEYANNYYPDTDDTAVALIALAPLRHDPKWKAKGIDEAIQLGVDWLIGMQSQGGGWGAFD |
| | | KDNNQKILTKIPFCDYGEALDPPSVDVTAHIIEAFGKLGISRNHPSMVQALDYIRREQEPSGPW |
| | | FGRWGVNYVYGTGAVLPALAAIGEDMTQPYIGRACDWLVAHQQADGGWGESCASYMDVSAVGRG |
| | | TTTASQTAWALMALLAANRPQDKDAIERGCMWLVERQSAGTWDEPEFTGTGFPGYGVGQTIKLN |
| | | DPALSQRLMQGPELSRAFMLRYGMYRHYFPLMALGRALRPQSHS |
| 47 | wt | MPTSLATAIDPKQLQQAIRASQDELFSQQYAEGYWWAELESNVTMTAEVILLHKIWGTEQRLPL |
| | TelSHC | AKAEQYLRNHQRDHGGWELFYGDGGDLSTSVEAYMGLRLLGVPETDPALVKARQFILARGGISK |
| | protein | TRIFTKLHLALIGCYDWRGIPSLPPWIMLLPEGSPFTIYEMSSWARSSTVPLLIVMDRKPVYGM |
| | | DPPITLDELYSEGRANVVWELPRQGDWRDVFIGLDRVFKLFETLNIHPLREQGLKAAEEWVLER |
| | | QEASGDWGGIIPAMLNSLLALRALDYAVDDPIVQRGMAAVDRFAIETETEYRVQPCVSPVWDTA |
| | | LVMRAMVDSGVAPDHPALVKAGEWLLSKQILDYGDWHIKNKKGRPGGWAFEFENRFYPDVDDTA |
| | | VVVMALHAVTLPNENLKRRAIERAVAWIASMQCRPGGWAAFDVDNDQDWLNGIPYGDLKAMIDP |
| | | NTADVTARVLEMVGRCQLAFDRVALDRALAYLRNEQEPEGCWFGRWGVNYLYGTSGVLTALSLV |
| | | APRYDRWRIRRAAEWLMQCQNADGGWGETCWSYHDPSLKGKGDSTASQTAWAIIGLLAAGDATG |
| | | DYATEAIERGIAYLLETQRPDGTWHEDYFTGTGFPCHFYLKYHYYQQHFPLTALGRYARWRNLL |
| | | AT |
| 48 | wt | MNMASRFSLKKILRSGSDTQGTNVNTLIQSGTSDIVRQKPAPQEPADLSALKAMGNSLTHTLSS |
| | ApaSHC1 | ACEWLMKQQKPDGHWVGSVGSNASMEAEWCLALWFLGLEDHPLRPRLGKALLEMQRPDGSWGTY |
| | protein | YGAGSGDINATVESYAALRSLGYAEDDPAVSKAAAWIISKGGLKNVRVFTRYWLALIGEWPWEK |
| | | TPNLPPEIIWFPDNFVESIYNFAQWARATMMPLAILSARRPSRPLRPQDRLDALFPGGRANFDY |
| | | ELPTKEGRDVIADFFRLADKGLHWLQSSFLKRAPSREAAIKYVLEWIIWHQDADGGWGGIQPPW |
| | | VYGLMALHGEGYQFHHPVMAKALDALNDPGWRHDKGDASWIQATNSPVWDTMLSLMALHDANAE |
| | | ERFTPEMDKALDWLLSRQVRVKGDWSVKLPNTEPGGWAFEYANDRYPDTDDTAVALIAIASCRN |
| | | RPEWQAKGVEEAIGRGVRWLVAMQSSCGGWGAFDKDNNKSILAKIPFCDEGEALDPPSVDVTAH |
| | | VLEAFGLLGLPRDLPCIQRGLAYIRKEQDPTGPWFGRWGVNYLYGTGAVLPALAALGEDMTQPY |
| | | ISKACDWLINCQQENGGWGESCASYMEVSSIGHGATTPSQTAWALMGLIAANRPQDYEAIAKGC |
| | | RYLIDLQEEDGSWNEEEFTGTGFPGYGVGQTIKLDDPAISKRLMQGAELSRAFMLRYDLYRQLF |
| | | PIIALSRASRLIKLGN |
| 49 | wt | MSPADISTKSSSFQRLDNMLPEAVSSACDWLIDQQKPDGHWVGPVESNACMEAQWCLALWELGQ |
| | GmoSHC | EDHPLRPRLAQALLEMQREDGSWGIYVGADHGDINTTVEAYAALRSMGYAADMPIMAKSAAWIQ |
| | protein | QKGGLRNVRVFTRYWLALIGEWPWDKTPNLPPEIIWLPDNFIFSIYNFAQWARATMMPLTILSA |
| | | RRPSRPLLPENRLDGLFPEGRENFDYELPVKGEEDLWGRFFRAADKGLHSLQSFPVRRFVPREA |
| | | AIRHVIEWIIRHQDADGGWGGIQPPWIYGLMALSVEGYPLHHPVLAKAMDALNDPGWRRDKGDA |
| | | SWIQATNSPVWDTMLAVLALHDAGAEDRYSPQMDKAIGWLLDRQVRVKGDWSIKLPDTEPGGWA |
| | | FEYANDKYPDTDDTAVALIALAGCRHRPEWRERDIEGAISRGVNWLLAMQSSSGGWGAFDKDNN |
| | | RSILTKIPFCDEGEALDPPSVDVTAHVLEAFGLLGISRNHPSVOKALAYIRSEQERNGAWFGRW |
| | | GVNYVYGTGAVLPALAAIGEDMTQPYIVRACDWLMSVQQENGGWGESCASYMDINAVGHGVATA |
| | | SQTAWALIGLLAAKRPKDREAIARGCQFLIERQEDGSWTEEEYTGTGFPGYGVGQAIKLDDPSL |
| | | PDRLLQGAELSRAFMLRYDLYRQYFPVMALSRARRMMKEDASAAA |
| 50 | BmeSHC# | ATGAACATCCTGCTGAAAGAAGTGCAGCTGGAAATTCAGCGTCGTATTGCATATCTGCGCCCGA |
| | 192_v70 | CCCAGAAAAATGATGGCAGTTTTCGTTATTGCTTTGAAACCGGTGTGATGCCGGATGCCTTTCT |
| | AA | GATTATGCTGCTGCGCACCTTTGATCTGGATAAAGAAGTTCTGATTAAGCAGCTGACCGAACGC |
| | | ATTGTTAGTCTGCAGAATGAAGATGGTCTGTGGACCCTGTTTGATGATGAAGAACATAATCTGA |
| | | GCGCAACCATTCAGGCATATACCGCCCTGCTGTATAGTGGTTATTATCAGAAAAATGACCGTAT |
| | | TCTGCGTAAAGCCGAACGTTATATTATTGATAGCGGTGGTATTAGCCGTGCACATTTTCTGACC |
| | | CGCTGGATGCTGAGCGTGAATGGCCTGTATGAATGGCCGAAACTGTTTTATCTGCCGCTGAGCC |
| | | TGCTGCTGGTTCCGACCTATGTTCCGCTGAATTTTTATGAACTGAGCGCCTATGCACGTATTCA |
| | | TTTTGTGCCGATGATGGTGGCAGGCAATAAGAAATTTTCACTGACCAGTCGTCATACCCCGAGC |
| | | CTGAGTCATCTGGATGTTCGTGAACAGAAACAGGAAAGCGAAGAAACCACCCAGGTTAGTCGCG |
| | | CAAGCATTTTTCTGGTGGATCATCTGAAACAGCTGGCAAGTCTGCCGAGTTATATTCATAAACT |
| | | GGGTTATCAGGCAGCCGAACGCTATATGCTGGAACGCATTGAAAAAGATGGCACCCTGTATAGT |
| | | TATGCAACCAGTACCTTTTTCATGATCTATGGCCTGCTGGCCCTGGGCTATAAAAAAGATAGCT |
| | | TTGTTATTCAGAAGGCAATTGATGGTATTTGTAGTCTGCTGAGTACCTGCAGTGGCCATGTTCA |
| | | TGTGGAAAATAGCACCAGTACCGTGTGGGATACCGCCCTGTTAAGCTATGCCCTGCAGGAAGCA |
| | | GGTGTTCCGCAGCAGGATCCGATGATTAAGGGTACCACCCGTTATCTGAAAAAACGTCAGCATA |
| | | CCAAACTGGGTGACTGGCAGTTTCATAATCCGAATACCGCCCCGGGTGGCTGGGGTTTTAGTGA |
| | | TATTAATACCAATAACCCGGATCTGGATGATACCAGTGCAGCCATTCGCGCCCTGAGTCGCCGC |
| | | GCTCAGACCGATACCGATTATCTGGAAAGTTGGCAGCGTGGTATTAATTGGCTGCTGAGTATGC |
| | | AGAATAAGGATGGTGGTTGGGCAGCCTTTGAAAAGAATACCGATAGCATTCTGTTTACCTATCT |
| | | GCCGTTAGAAAATGCAAAAGATGCCGCAACCGATCCGGCCACCGCCGATCTGACCGGCCGTGTT |
| | | CTGGAATGCCTGGGTAATTTTGCAGGCATGAATAAGAGTCATCCGAGCATTAAGGCCGCAGTGA |
| | | AATGGCTGTTTGATCATCAGCTGGATAATGGTAGTTGGTGTGGCCGTTGGGGTGTGTGTTATAT |
| | | CTATGGTACCTGGGCAGCCATTACCGGTCTGCGTGCAGTGGGCGTGAGTGCAAGCGATCCGCGT |
| | | ATTATTAAGGCAATTAATTGGTTAAAGAGCATCCAGCAGGAAGATGGCGGTTTTGGCGAAAGCT |
| | | GTTATAGTGCAAGTCTGAAAAAATATGTGCCGCTGAGTTTTAGTACCCCGAGCCAGACCGCCTG |
| | | GGCACTGGATGCACTGATGACCATTTGTCCGCTGAAAGATCAGAGTGTGGAAAAAGGCATTAAG |
| | | TTTCTGCTGAATCCGAATCTGACCGAACAGCAGACCCATTATCCGACCGGTATTGGTCTGCCGG |
| | | GTCAGTTTTATATTCAGTATCATAGTTACAACGACATCTTTCCGCTGCTGGCACTGGCCCATTA |
| | | TGCCAAAAAACATAGCAGCTAA |
| 51 | BmeSHC# | MNILLKEVQLEIQRRIAYLRPTQKNDGSFRYCFETGVMPDAFLIMLLRTEDLDKEVLIKQLTER |
| | 192_v70 | IVSLQNEDGLWTLEDDEEHNLSATIQAYTALLYSGYYQKNDRILRKAERYIIDSGGISRAHFLT |
| | AA | RWMLSVNGLYEWPKLFYLPLSLLLVPTYVPLNFYELSAYARIHEVPMMVAGNKKFSLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGESDINTNNPDLDDTSAAIRALSRR |
| | | AQTDTDYLESWQRGINWLLSMQNKDGGWAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | ELLNPNLTEQQTHYPTGIGLPGQFYIQYHSYNDIFPLLALAHYAKKHSS |
| 52 | BmeSHC# | ATGAACATCCTGCTGAAAGAAGTGCAGCTGGAAATTCAGCGTCGTATTGCATATCTGCGCCCGA |
| | 192_v71 | CCCAGAAAAATGATGGCAGTTTTCGTTATTGCTTTGAAACCGGTGTGATGCCGGATGCCTTTCT |
| | DNA | GATTATGCTGCTGCGCACCTTTGATCTGGATAAAGAAGTTCTGATTAAGCAGCTGACCGAACGC |
| | | ATTGTTAGTCTGCAGAATGAAGATGGTCTGTGGACCCTGTTTGATGATGAAGAACATAATCTGA |
| | | GCGCAACCATTCAGGCATATACCGCCCTGCTGTATAGTGGTTATTATCAGAAAAATGACCGTAT |
| | | TCTGCGTAAAGCCGAACGTTATATTATTGATAGCGGTGGTATTAGCCGTGCACATTTTCTGACC |
| | | CGCTGGATGCTGAGCGTGAATGGCCTGTATGAATGGCCGAAACTGTTTTATCTGCCGCTGAGCC |
| | | TGCTGCTGGTTCCGACCTATGTTCCGCTGAATTTTTATGAACTGAGCGCCTATGCACGTATTCA |
| | | TTTTGTGCCGATGATGGTGGCAGGCAATAAGAAATTTTCACTGACCAGTCGTCATACCCCGAGC |
| | | CTGAGTCATCTGGATGTTCGTGAACAGAAACAGGAAAGCGAAGAAACCACCCAGGTTAGTCGCG |
| | | CAAGCATTTTTCTGGTGGATCATCTGAAACAGCTGGCAAGTCTGCCGAGTTATATTCATAAACT |
| | | GGGTTATCAGGCAGCCGAACGCTATATGCTGGAACGCATTGAAAAAGATGGCACCCTGTATAGT |
| | | TATGCAACCAGTACCTTTTTCATGATCTATGGCCTGCTGGCCCTGGGCTATAAAAAAGATAGCT |
| | | TTGTTATTCAGAAGGCAATTGATGGTATTTGTAGTCTGCTGAGTACCTGCAGTGGCCATGTTCA |
| | | TGTGGAAAATAGCACCAGTACCGTGTGGGATACCGCCCTGTTAAGCTATGCCCTGCAGGAAGCA |
| | | GGTGTTCCGCAGCAGGATCCGATGATTAAGGGTACCACCCGTTATCTGAAAAAACGTCAGCATA |
| | | CCAAACTGGGTGACTGGCAGTTTCATAATCCGAATACCGCCCCGGGTGGCTGGGGTTTTAGTGA |
| | | TATTAATACCAATAACCCGGATCTGGATGATACCAGTGCAGCCATTCGCGCCCTGAGTCGCCGC |
| | | GCTCAGACCGATACCGATTATCTGGAAAGTTGGCAGCGTGGTATTAATTGGCTGCTGAGTATGC |
| | | AGAATAAGGATGGTGGTTTTGCAGCCTTTGAAAAGAATACCGATAGCATTCTGTTTACCTATCT |
| | | GCCGTTAGAAAATGCAAAAGATGCCGCAACCGATCCGGCCACCGCCGATCTGACCGGCCGTGTT |
| | | CTGGAATGCCTGGGTAATTTTGCAGGCATGAATAAGAGTCATCCGAGCATTAAGGCCGCAGTGA |
| | | AATGGCTGTTTGATCATCAGCTGGATAATGGTAGTTGGTGTGGCCGTTGGGGTGTGTGTTATAT |
| | | CTATGGTACCTGGGCAGCCATTACCGGTCTGCGTGCAGTGGGCGTGAGTGCAAGCGATCCGCGT |
| | | ATTATTAAGGCAATTAATTGGTTAAAGAGCATCCAGCAGGAAGATGGCGGTTGGGGCGAAAGCT |
| | | GTTATAGTGCAAGTCTGAAAAAATATGTGCCGCTGAGTTTTAGTACCCCGAGCCAGACCGCCTG |
| | | GGCACTGGATGCACTGATGACCATTTGTCCGCTGAAAGATCAGAGTGTGGAAAAAGGCATTAAG |
| | | TTTCTGCTGAATCCGAATCTGACCGAACAGCAGACCCATTATCCGACCGGTATTGGTCTGCCGG |
| | | GTCAGTTTTATATTCAGTATCATAGTTACAACGACATCTTTCCGCTGCTGGCACTGGCCCATTA |
| | | TGCCAAAAAACATAGCAGCTAA |
| 53 | BmeSHC# | MNILLKEVQLEIQRRIAYLRPTQKNDGSFRYCFETGVMPDAFLIMLLRTEDLDKEVLIKQLTER |
| | 192_v71 | IVSLQNEDGLWTLEDDEEHNLSATIQAYTALLYSGYYQKNDRILRKAERYIIDSGGISRAHFLT |
| | AA | RWMLSVNGLYEWPKLFYLPLSLLLVPTYVPLNFYELSAYARIHFVPMMVAGNKKFSLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGFAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGWGESCYSASLKKYVPLSFSTPSQTAWALDALMTICPLKDQSVEKGIK |
| | | ELLNPNLTEQQTHYPTGIGLPGQFYIQYHSYNDIFPLLALAHYAKKHSS |
| 54 | BmeSHC# | ATGAACATCCTGCTGAAAGAAGTGCAGCTGGAAATTCAGCGTCGTATTGCATATCTGCGCCCGA |
| | 192_v72 | CCCAGAAAAATGATGGCAGTTGGCGTTATTGCTTTGAAACCGGTGTGATGCCGGATGCCTTTCT |
| | DNA | GATTATGCTGCTGCGCACCTTTGATCTGGATAAAGAAGTTCTGATTAAGCAGCTGACCGAACGC |
| | | ATTGTTAGTCTGCAGAATGAAGATGGTCTGTGGACCCTGTTTGATGATGAAGAACATAATCTGA |
| | | GCGCAACCATTCAGGCATATACCGCCCTGCTGTATAGTGGTTATTATCAGAAAAATGACCGTAT |
| | | TCTGCGTAAAGCCGAACGTTATATTATTGATAGCGGTGGTATTAGCCGTGCACATTTTCTGACC |
| | | CGCTGGATGCTGAGCGTGAATGGCCTGTATGAATGGCCGAAACTGTTTTATCTGCCGCTGAGCC |
| | | TGCTGCTGGTTCCGACCTATGTTCCGCTGAATTTTTATGAACTGAGCGCCTATGCACGTATTCA |
| | | TTTTGTGCCGATGATGGTGGCAGGCAATAAGAAATTTTCACTGACCAGTCGTCATACCCCGAGC |
| | | CTGAGTCATCTGGATGTTCGTGAACAGAAACAGGAAAGCGAAGAAACCACCCAGGTTAGTCGCG |
| | | CAAGCATTTTTCTGGTGGATCATCTGAAACAGCTGGCAAGTCTGCCGAGTTATATTCATAAACT |
| | | GGGTTATCAGGCAGCCGAACGCTATATGCTGGAACGCATTGAAAAAGATGGCACCCTGTATAGT |
| | | TATGCAACCAGTACCTTTTTCATGATCTATGGCCTGCTGGCCCTGGGCTATAAAAAAGATAGCT |
| | | TTGTTATTCAGAAGGCAATTGATGGTATTTGTAGTCTGCTGAGTACCTGCAGTGGCCATGTTCA |
| | | TGTGGAAAATAGCACCAGTACCGTGTGGGATACCGCCCTGTTAAGCTATGCCCTGCAGGAAGCA |
| | | GGTGTTCCGCAGCAGGATCCGATGATTAAGGGTACCACCCGTTATCTGAAAAAACGTCAGCATA |
| | | CCAAACTGGGTGACTGGCAGTTTCATAATCCGAATACCGCCCCGGGTGGCTGGGGTTTTAGTGA |
| | | TATTAATACCAATAACCCGGATCTGGATGATACCAGTGCAGCCATTCGCGCCCTGAGTCGCCGC |
| | | GCTCAGACCGATACCGATTATCTGGAAAGTTGGCAGCGTGGTATTAATTGGCTGCTGAGTATGC |
| | | AGAATAAGGATGGTGGTTGGGCAGCCTTTGAAAAGAATACCGATAGCATTCTGTTTACCTATCT |
| | | GCCGTTAGAAAATGCAAAAGATGCCGCAACCGATCCGGCCACCGCCGATCTGACCGGCCGTGTT |
| | | CTGGAATGCCTGGGTAATTTTGCAGGCATGAATAAGAGTCATCCGAGCATTAAGGCCGCAGTGA |
| | | AATGGCTGTTTGATCATCAGCTGGATAATGGTAGTTGGTGTGGCCGTTGGGGTGTGTGTTATAT |
| | | CTATGGTACCTGGGCAGCCATTACCGGTCTGCGTGCAGTGGGCGTGAGTGCAAGCGATCCGCGT |
| | | ATTATTAAGGCAATTAATTGGTTAAAGAGCATCCAGCAGGAAGATGGCGGTTTTGGCGAAAGCT |
| | | GTTATAGTGCAAGTCTGAAAAAATATGTGCCGCTGAGTTTTAGTACCCCGAGCCAGACCGCCTG |
| | | GGCACTGGATGCACTGATGACCATTTGTCCGCTGAAAGATCAGAGTGTGGAAAAAGGCATTAAG |
| | | TTTCTGCTGAATCCGAATCTGACCGAACAGCAGACCCATTATCCGACCGGTATTGGTCTGCCGG |
| | | GTCAGTTTTATATTCAGTATCATAGTTACAACGACATCTTTCCGCTGCTGGCACTGGCCCATTA |
| | | TGCCAAAAAACATAGCAGCTAA |
| 55 | BmeSHC# | MNILLKEVQLEIQRRIAYLRPTQKNDGSWRYCFETGVMPDAFLIMLLRTEDLDKEVLIKQLTER |
| | 192_v72 | IVSLQNEDGLWTLEDDEEHNLSATIQAYTALLYSGYYQKNDRILRKAERYIIDSGGISRAHFLT |
| | AA | RWMLSVNGLYEWPKLFYLPLSLLLVPTYVPLNFYELSAYARIHFVPMMVAGNKKESLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGWAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGFGESCYSASLKKYVPLSFSTPSQTAWALDALMTICPLKDQSVEKGIK |
| | | ELLNPNLTEQQTHYPTGIGLPGQFYIQYHSYNDIFPLLALAHYAKKHSS |
| 56 | BmeSHC# | ATGAACATCCTGCTGAAAGAAGTGCAGCTGGAAATTCAGCGTCGTATTGCATATCTGCGCCCGA |
| | 192_v73 | CCCAGAAAAATGATGGCAGTTGGCGTTATTGCTTTGAAACCGGTGTGATGCCGGATGCCTTTCT |
| | DNA | GATTATGCTGCTGCGCACCTTTGATCTGGATAAAGAAGTTCTGATTAAGCAGCTGACCGAACGC |
| | | ATTGTTAGTCTGCAGAATGAAGATGGTCTGTGGACCCTGTTTGATGATGAAGAACATAATCTGA |
| | | GCGCAACCATTCAGGCATATACCGCCCTGCTGTATAGTGGTTATTATCAGAAAAATGACCGTAT |
| | | TCTGCGTAAAGCCGAACGTTATATTATTGATAGCGGTGGTATTAGCCGTGCACATTTTCTGACC |
| | | CGCTGGATGCTGAGCGTGAATGGCCTGTATGAATGGCCGAAACTGTTTTATCTGCCGCTGAGCC |
| | | TGCTGCTGGTTCCGACCTATGTTCCGCTGAATTTTTATGAACTGAGCGCCTATGCACGTATTCA |
| | | TTTTGTGCCGATGATGGTGGCAGGCAATAAGAAATTTTCACTGACCAGTCGTCATACCCCGAGC |
| | | CTGAGTCATCTGGATGTTCGTGAACAGAAACAGGAAAGCGAAGAAACCACCCAGGTTAGTCGCG |
| | | CAAGCATTTTTCTGGTGGATCATCTGAAACAGCTGGCAAGTCTGCCGAGTTATATTCATAAACT |
| | | GGGTTATCAGGCAGCCGAACGCTATATGCTGGAACGCATTGAAAAAGATGGCACCCTGTATAGT |
| | | TATGCAACCAGTACCTTTTTCATGATCTATGGCCTGCTGGCCCTGGGCTATAAAAAAGATAGCT |
| | | TTGTTATTCAGAAGGCAATTGATGGTATTTGTAGTCTGCTGAGTACCTGCAGTGGCCATGTTCA |
| | | TGTGGAAAATAGCACCAGTACCGTGTGGGATACCGCCCTGTTAAGCTATGCCCTGCAGGAAGCA |
| | | GGTGTTCCGCAGCAGGATCCGATGATTAAGGGTACCACCCGTTATCTGAAAAAACGTCAGCATA |
| | | CCAAACTGGGTGACTGGCAGTTTCATAATCCGAATACCGCCCCGGGTGGCTGGGGTTTTAGTGA |
| | | TATTAATACCAATAACCCGGATCTGGATGATACCAGTGCAGCCATTCGCGCCCTGAGTCGCCGC |
| | | GCTCAGACCGATACCGATTATCTGGAAAGTTGGCAGCGTGGTATTAATTGGCTGCTGAGTATGC |
| | | AGAATAAGGATGGTGGTTGGGCAGCCTTTGAAAAGAATACCGATAGCATTCTGTTTACCTATCT |
| | | GCCGTTAGAAAATGCAAAAGATGCCGCAACCGATCCGGCCACCGCCGATCTGACCGGCCGTGTT |
| | | CTGGAATGCCTGGGTAATTTTGCAGGCATGAATAAGAGTCATCCGAGCATTAAGGCCGCAGTGA |
| | | AATGGCTGTTTGATCATCAGCTGGATAATGGTAGTTGGTGTGGCCGTTGGGGTGTGTGTTATAT |
| | | CTATGGTACCTGGGCAGCCATTACCGGTCTGCGTGCAGTGGGCGTGAGTGCAAGCGATCCGCGT |
| | | ATTATTAAGGCAATTAATTGGTTAAAGAGCATCCAGCAGGAAGATGGCGGTTGGGGCGAAAGCT |
| | | GTTATAGTGCAAGTCTGAAAAAATATGTGCCGCTGAGTTTTAGTACCCCGAGCCAGACCGCCTG |
| | | GGCACTGGATGCACTGATGACCATTTGTCCGCTGAAAGATCAGAGTGTGGAAAAAGGCATTAAG |
| | | TTTCTGCTGAATCCGAATCTGACCGAACAGCAGACCCATTATCCGACCGGTATTGGTCTGCCGG |
| | | GTCAGTTTTATATTCAGTATCATAGTTACAACGACATCTTTCCGCTGCTGGCACTGGCCCATTA |
| | | TGCCAAAAAACATAGCAGCTAA |
| 57 | BmeSHC# | MNILLKEVQLEIQRRIAYLRPTQKNDGSWRYCFETGVMPDAFLIMLLRTFDLDKEVLIKQLTER |
| | 192_v73 | IVSLQNEDGLWTLEDDEEHNLSATIQAYTALLYSGYYQKNDRILRKAERYIIDSGGISRAHFLT |
| | AA | RWMLSVNGLYEWPKLFYLPLSLLLVPTYVPLNFYELSAYARIHFVPMMVAGNKKFSLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGFSDINTNNPDLDDTSAAIRALSRR |
| | | AQTDTDYLESWQRGINWLLSMQNKDGGWAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGWGESCYSASLKKYVPLSFSTPSQTAWALDALMTICPLKDQSVEKGIK |
| | | ELLNPNLTEQQTHYPTGIGLPGQFYIQYHSYNDIFPLLALAHYAKKHSS |
| 58 | BmeSHC# | ATGAACATCCTGCTGAAAGAAGTGCAGCTGGAAATTCAGCGTCGTATTGCATATCTGCGCCCGA |
| | 192_v75 | CCCAGAAAAATGATGGCAGTTTTCGTTATTGCTTTGAAACCGGTGTGATGCCGGATGCCTTTCT |
| | DNA | GATTATGCTGCTGCGCACCTTTGATCTGGATAAAGAAGTTCTGATTAAGCAGCTGACCGAACGC |
| | | ATTGTTAGTCTGCAGAATGAAGATGGTCTGTGGACCCTGTTTGATGATGAAGAACATAATCTGA |
| | | GCGCAACCATTCAGGCATATACCGCCCTGCTGTATAGTGGTTATTATCAGAAAAATGACCGTAT |
| | | TCTGCGTAAAGCCGAACGTTATATTATTGATAGCGGTGGTATTAGCCGTGCACATTTTCTGACC |
| | | CGCTGGATGCTGAGCGTGAATGGCCTGTATGAATGGCCGAAACTGTTTTATCTGCCGCTGAGCC |
| | | TGCTGCTGGTTCCGACCTATGTTCCGCTGAATTTTTATGAACTGAGCGCCTATGCACGTATTCA |
| | | TTTTGTGCCGATGATGGTGGCAGGCAATAAGAAATTTTCACTGACCAGTCGTCATACCCCGAGC |
| | | CTGAGTCATCTGGATGTTCGTGAACAGAAACAGGAAAGCGAAGAAACCACCCAGGTTAGTCGCG |
| | | CAAGCATTTTTCTGGTGGATCATCTGAAACAGCTGGCAAGTCTGCCGAGTTATATTCATAAACT |
| | | GGGTTATCAGGCAGCCGAACGCTATATGCTGGAACGCATTGAAAAAGATGGCACCCTGTATAGT |
| | | TATGCAACCAGTACCTTTTTCATGATCTATGGCCTGCTGGCCCTGGGCTATAAAAAAGATAGCT |
| | | TTGTTATTCAGAAGGCAATTGATGGTATTTGTAGTCTGCTGAGTACCTGCAGTGGCCATGTTCA |
| | | TGTGGAAAATAGCACCAGTACCGTGTGGGATACCGCCCTGTTAAGCTATGCCCTGCAGGAAGCA |
| | | GGTGTTCCGCAGCAGGATCCGATGATTAAGGGTACCACCCGTTATCTGAAAAAACGTCAGCATA |
| | | CCAAACTGGGTGACTGGCAGTTTCATAATCCGAATACCGCCCCGGGTGGCTGGGGTTTTAGTGA |
| | | TATTAATACCAATAACCCGGATCTGGATGATACCAGTGCAGCCATTCGCGCCCTGAGTCGCCGC |
| | | GCTCAGACCGATACCGATTATCTGGAAAGTTGGCAGCGTGGTATTAATTGGCTGCTGAGTATGC |
| | | AGAATAAGGATGGTGGTTGGGCAGCCTTTGAAAAGAATACCGATAGCATTCTGTTTACCTATCT |
| | | GCCGTTAGAAAATGCAAAAGATGCCGCAACCGATCCGGCCACCGCCGATCTGACCGGCCGTGTT |
| | | CTGGAATGCCTGGGTAATTTTGCAGGCATGAATAAGAGTCATCCGAGCATTAAGGCCGCAGTGA |
| | | AATGGCTGTTTGATCATCAGCTGGATAATGGTAGTTGGTGTGGCCGTTGGGGTGTGTGTTATAT |
| | | CTATGGTACCTGGGCAGCCATTACCGGTCTGCGTGCAGTGGGCGTGAGTGCAAGCGATCCGCGT |
| | | ATTATTAAGGCAATTAATTGGTTAAAGAGCATCCAGCAGGAAGATGGCGGTTGGGGCGAAAGCT |
| | | GTTATAGTGCAAGTCTGAAAAAATATGTGCCGCTGAGTTTTAGTACCCCGAGCCAGACCGCCTG |
| | | GGCACTGGATGCACTGATGACCATTTGTCCGCTGAAAGATCAGAGTGTGGAAAAAGGCATTAAG |
| | | TTTCTGCTGAATCCGAATCTGACCGAACAGCAGACCCATTATCCGACCGGTATTGGTCTGCCGG |
| | | GTCAGTTTTATATTCAGTATCATAGTTACAACGACATCTTTCCGCTGCTGGCACTGGCCCATTA |
| | | TGCCAAAAAACATAGCAGCTAA |
| 59 | BmeSHC# | MNILLKEVQLEIQRRIAYLRPTQKNDGSFRYCFETGVMPDAFLIMLLRTEDLDKEVLIKQLTER |
| | 192_v75 | IVSLQNEDGLWTLEDDEEHNLSATIQAYTALLYSGYYQKNDRILRKAERYIIDSGGISRAHFLT |
| | AA | RWMLSVNGLYEWPKLFYLPLSLLLVPTYVPLNFYELSAYARIHFVPMMVAGNKKFSLTSRHTPS |
| | | LSHLDVREQKQESEETTQVSRASIFLVDHLKQLASLPSYIHKLGYQAAERYMLERIEKDGTLYS |
| | | YATSTFFMIYGLLALGYKKDSFVIQKAIDGICSLLSTCSGHVHVENSTSTVWDTALLSYALQEA |
| | | GVPQQDPMIKGTTRYLKKRQHTKLGDWQFHNPNTAPGGWGESDINTNNPDLDDTSAAIRALSRR |
| | | AQTDTDYLESWQRGINWLLSMQNKDGGWAAFEKNTDSILFTYLPLENAKDAATDPATADLTGRV |
| | | LECLGNFAGMNKSHPSIKAAVKWLFDHQLDNGSWCGRWGVCYIYGTWAAITGLRAVGVSASDPR |
| | | IIKAINWLKSIQQEDGGWGESCYSASLKKYVPLSFSTPSQTAWALDALMTICPLKDQSVEKGIK |
| | | ELLNPNLTEQQTHYPTGIGLPGQFYIQYHSYNDIFPLLALAHYAKKHSS |

### General Information

Unless stated otherwise, all technical and scientific terms used herein have the same meaning as customarily and ordinarily understood by a person of ordinary skill in the art to which this disclosure belongs, and read in view of this disclosure.

### Sequence Identity

In the context of the disclosure, a nucleic acid molecule such as a nucleic acid molecule encoding an SHC enzyme as described herein is represented by a nucleic acid or nucleotide sequence which encodes an SHC enzyme as described herein.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 30%, 40%, 50%, 60%, 70%, 80%, 90%, 95% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 30%, 40%, 50%, 60%, 70%, 80%, 90%, 95% or 99% amino acid identity or similarity with an amino acid sequence encoded by a nucleotide sequence SEQ ID NO: X.

Another preferred level of sequence identity or similarity is 30%. Another preferred level of sequence identity or similarity is 40%. Another preferred level of sequence identity or similarity is 50%. Another preferred level of sequence identity or similarity is 60%. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 30%, 40%, 50%, 60%, 70%, 80%, 90%, 95% or 99% sequence identity or similarity with amino acid sequence SEQ ID NO: Y. Another preferred level of sequence identity or similarity is 30%. Another preferred level of sequence identity or similarity is 40%. Another preferred level of sequence identity or similarity is 50%. Another preferred level of sequence identity or similarity is 60%. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acids (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively, percentage similarity or identity may be determined by searching against public databases, using algorithms such as FASTA, BLAST, etc. Thus, the nucleic acid and protein sequences of some embodiments of the present disclosure can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the BLASTN program, score=100, wordlength=12 to obtain nucleotide sequences homologous to oxidoreductase nucleic acid molecules of the disclosure. BLAST protein searches can be performed with the BLASTx program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nim.nih.gov/.

Sequence matching analysis may be supplemented by established homology mapping techniques like Shuffle-LAGAN (Brudno M., Bioinformatics 2003b, 19 Suppl 1:154-162) or Markov random fields. Optionally, in determining the degree of amino acid similarity, the skilled person may also take into account so-called conservative amino acid substitutions as discussed earlier herein.

### Gene or Coding Sequence

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site. A chimeric or recombinant gene is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter or one-letter symbols. Three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (IIe) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments described herein are capable of operation in other sequences than described or illustrated herein.

The word “about” or “approximately” when used in association with a numerical value (e.g. about 10) preferably means that the value may be the given value (of 10) more or less 1% of the value.

In the context of the present disclosure, the term “and/or” is understood to mean that all members of a group connected by the term “and/or” are represented both cumulatively with respect to each other in any combination, and alternatively with respect to each other. Exemplarily, for the expression “A, B and/or C”, the following disclosure is to be understood thereunder: i) (A or B or C), or ii) (A and B), or iii) (A and C), or iv) (B and C), or v) (A and B and C), or vi) (A and B or C), or vii) (A or B and C), or viii) (A and C or B).

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The disclosure is not limited by the methods, protocols, and materials described herein. One skilled in the art will recognize many methods, protocols, and materials similar or equivalent to those described herein, which could be used in the practices described herein. Indeed, the present disclosure is in no way limited to the methods and materials described. It is also understood that the disclosure encompasses the generalization of aspects of the following examples to the preceding disclosure.

The present disclosure is further described by the following examples which should not be construed as limiting its scope.

### DESCRIPTION OF THE FIGURES

FIG. 1. Reaction scheme for the production of a compound of formula (II). For the compounds, R is optionally selected from H and a C₁-C₄ alkyl.

FIG. 2. SHC enzyme activity with selected SHC variants. E,Z-HFA conversion is indicated relative to conversion with BmeSHC as tested during library screening and selection of improved variants (2 g/l E,Z-HFA, cells to OD_{650 nm} 10, 0.005% SDS, 50 mM succinate/NaOH buffer pH 5.2, 35° C., 250 rpm, 24 h).

FIG. 3. SHC enzyme activity with selected SHC variants. Reaction conditions were the same as discussed in FIG. 2. Biocatalysts used were produced in fermentations.

FIG. 4. SHC enzyme activity with selected SHC variants. E,Z-HFA conversion is indicated relative to conversion with wt BmeSHC as tested during mutations study and selection of improved variants (4 g/l E,Z-HFA, cells to an OD_{650 nm} of 10, 0.004% SDS, 50 mM succinate/NaOH buffer pH 5.2, 35° C., 250 rpm, 24 h).

FIG. 5. SHC enzyme activity with selected SHC variants. Reaction conditions were the same as discussed in FIG. 4. Biocatalysts used were produced in fermentations.

FIG. 6. SHC enzyme activity with selected SHC variants. E,Z-HFA conversion is indicated relative to conversion with wt BmeSHC (4 g/l E,Z-HFA, cells to an OD_{650 nm} of 10, 0.004% SDS, 50 mM succinate/NaOH buffer pH 5.2, 35° C., 250 rpm, 24 h).

FIG. 7. Relative activity of wt and variant BmeSHC enzymes. Reactions were run with 135 g/l E,Z-HFA and 182 g/l cells, at T, pH and SDS (SDS: cells ratio) conditions defined as optimal for each of the variants. Conversion with wt BmeSHC is set as reference (100).

FIG. 8. Relative activity of BmeSHC #192 and BmeSHC #192 variants. Reactions were run with 135 g/l E,Z HFA and 182 g/l cells, at T, pH and SDS ([SDS]: [cells] ratio) conditions individually defined as optimal for each of the variants tested. Conversion with BmeSHC #192 is set as reference to 100.

FIG. 9. Relative activity of BmeSHC #192 and BmeSHC #192 variants. Reactions were run with 100 g/l E,Z-HFA and 100 g/l cells, at T, pH and SDS ([SDS]: [cells] ratio) conditions individually defined as optimal for each of the variants tested. Conversion with BmeSHC #192 is set as reference to 100.

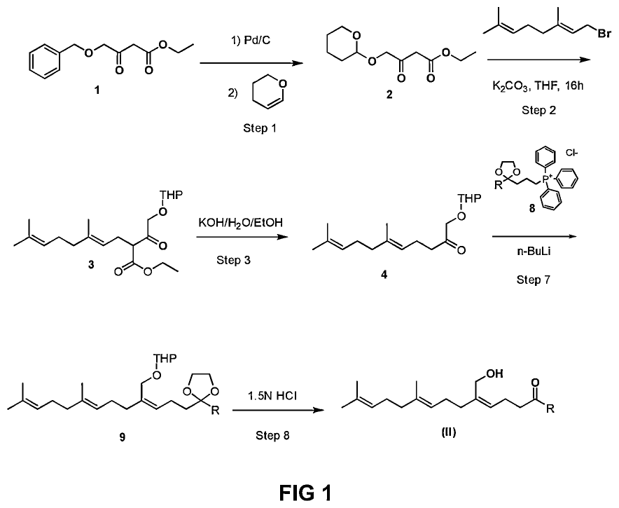

### EXAMPLES

### Example 1: SHC Enzyme Evolution: Library Screening, BmeSHC Variants, New Mutations

An enzyme evolution program was done using the gene coding for the Bacillus megaterium SHC enzyme as a template. A library of about 11′300 SHC variants was produced and screened for variants showing an increased ability to cyclize E,Z-Hydroxyfarnesylacetone (E,Z-HFA) to (+)-amberketal. Gene expression for SHC production was done in E. coli MC1061 (DE3): 0.5 ml cultures in auto-inducing medium, incubated at 37° C. for 2 h followed by 22 h at 20° C. (250 rpm). Cells were collected by centrifugation and washed with 50 mM succinic acid/NaOH buffer pH 5.2.

SHC activity screening was done in 96 deep-well plates. 0.5 ml reactions were run in 50 mM succinic acid/NaOH buffer pH 5.2. They contained 2 g/l E,Z-HFA and 0.004% sodium dodecyl sulfate (SDS), cells that had produced the SHC variants to an OD_{650 nm} of 10. Reactions were run for 3 hours at 35° C. under constant agitation (orbital shaking, 250 rpm), solvent-extracted for GC-FID analysis for the determination of E,Z-HFA conversion to (+)-amberketal as described in Example 7.

316 of the approx. 11′300 variants produced were chosen for validation. The conditions described above for library screening were applied.

82 of the 316 variants above were chosen for confirmation at larger scale. 20 ml cultures were run in auto-inducing medium following the cultivation scheme and cell harvest described above. SHC activity was assayed in the setup described above. The reactions contained 2 or 4 g/l E,Z-HFA, cells to an OD_{650 nm} of 10 or 20, 0.01 or 0.005% SDS depending on cell concentration (constant SDS/cells ratio). Reactions were incubated for 2, 4, or 6 h at 35° C. (250 rpm) prior to solvent extraction for GC-FID analysis for determining E,Z-HFA conversion to (+)-amberketal as described in Example 7.

23 of the above 82 variants were selected for a final confirmation step. 20 ml cultures were run in auto-inducing medium (incubation for 2 h at 37° C., then for 22 h at 20° C. (180 rpm)). Cells were collected by centrifugation, washed, and concentrated to an OD_{650 nm} of 200 in 50 mM succinic acid/NaOH buffer pH 5.2. Activity was assayed in 96 deepwell plates. Reactions in 50 mM succinic acid/NaOH buffer pH 5.2 contained 2, 4 or 8 g/l E,Z-HFA with cells to an OD_{650 nm} of 5 or 10, and 0.0025 or 0.005% SDS depending on the cell concentration (constant SDS/cells ratio). Reactions were sampled over time, solvent-extracted and analyzed by gas chromatography for determining E,Z-HFA conversion to (+)-amberketal as described in Example 7.

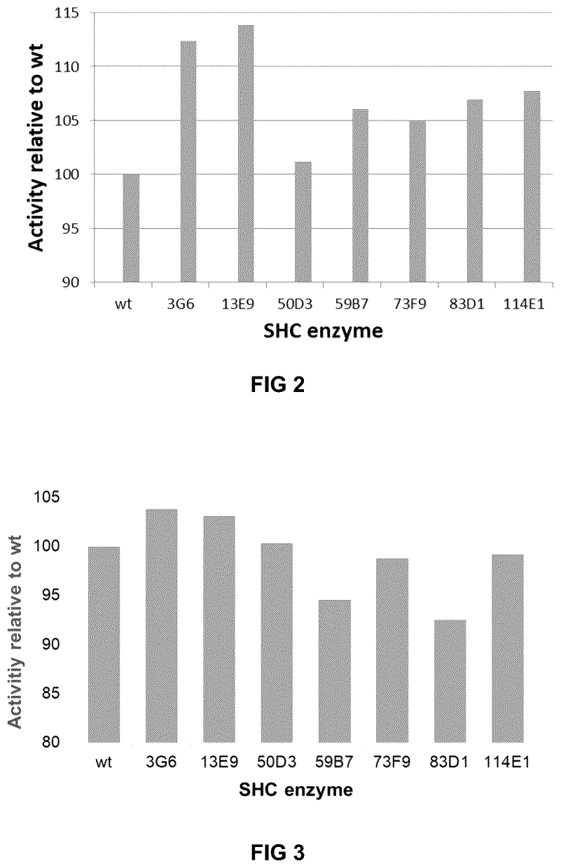

7 variants with improved E,Z-HFA cyclization activity depending on the conditions applied for activity testing (substrate concentration, reaction time) uncovered the mutations listed in Table 2. These variants were selected for in-depth characterization. Their activity (E,Z-HFA conversion relative to conversion with wt BmeSHC) in reactions containing 2 g/l EZHFA and cells to an OD_{650 nm} of 10 is shown in FIG. 2. The activity of these variants when produced by fermentation is shown in FIG. 3. The result indicated that the activity of the biocatalyst was strongly dependent on how biocatalysts were produced (flask cultivation vs. fermentation, auto-inducing medium vs. minimal medium)

TABLE 2
Mutations in selected BmeSHC variants

| | | | | | | SEQ ID NO | SEQ ID NO |
|---|---|---|---|---|---|---|---|
| SHCvariant | Mutations | | | | | (DNA) | (AA) |
| 3G6 | I2N | T35A | A355T | L539H | | 3 | 4 |
| 13E9 | T166A | | | | | 7 | 8 |
| 50D3 | I116T | E221V | S212R | L317M | E585A | 9 | 10 |
| 59B7 | Y483C | | | | | 5 | 6 |
| 73F9 | I399V | | | | | 11 | 12 |
| 83D1 | L5P | | | | | 13 | 14 |
| 114E1 | S382T | | | | | 15 | 16 |

### Example 2: Mutations Study 1

A mutations study was done to determine the impact of the mutations of variants 3G6 and 50D3 on E,Z-HFA cyclization to (+)-amberketal. All possible combinations of 3G6 and 50D3 mutations were studied, alone and associated with Y483C, L5P and Y483C+L5P mutations. 176 additional variants were constructed and tested for their E,Z-HFA to (+)-amberketal cyclization activity.

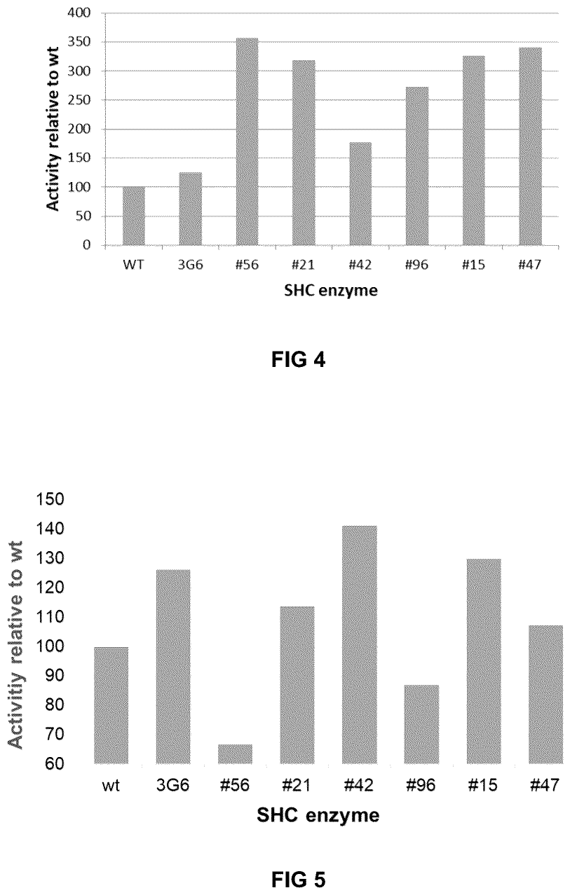

Cultivation and gene expression was done in microtiter plates as described for library screening (Example 1). SHC activity was assayed in 0.5 ml reaction with 2 and 4 g/l E,Z-HFA; cells to an OD_{650 nm} of 10, 0.004% SDS in 50 mM succinic acid/NaOH buffer pH 5.2 (250 rpm). Reactions were incubated for 3 or 6 hours prior to solvent extraction and GC analysis as described in Example 7. The mutations in selected variants are shown in Table 3, the activity of the variants (E,Z-HFA conversion relative to wt BmeSHC after 24 h of reaction) is shown in FIG. 4. The activity of these biocatalysts produced by fermentation is shown in FIG. 5. The result indicated that the activity of the biocatalyst was strongly dependent on how the cells were produced.

The mutations combination study allowed to identify five beneficial mutations: I2N, Y483C, L539H, L5P, T35A.

TABLE 3
Mutations in selected BmeSHC variants

| SHC | | | | | | SEQ ID NO | SEQ ID NO |
|---|---|---|---|---|---|---|---|
| variant | Mutations | | | | | (DNA) | (AA) |
| 3G6 | I2N | T35A | A355T | L539H | | 3 | 4 |
| #15 | I2N | Y483C | | | | 17 | 18 |
| #21 | I2N | Y483C | L539H | | | 19 | 20 |
| #42 | I2N | L5P | T35A | L539H | | 21 | 22 |
| #47 | I2N | L5P | T35A | Y483C | | 23 | 24 |
| #56 | I2N | L5P | T35A | Y483C | L539H | 25 | 26 |
| #96 | E211V | S212R | Y483C | | | 27 | 28 |

### Example 3: Mutations Study 2

The mutations identified as beneficial during mutations study 1 (Example 2) were combined with mutations E211V and T166A also identified as beneficial. E211V and/or T166A were added to SHC variants #15, #21, #42, #47, #56, and #96:21 additional variants were constructed.

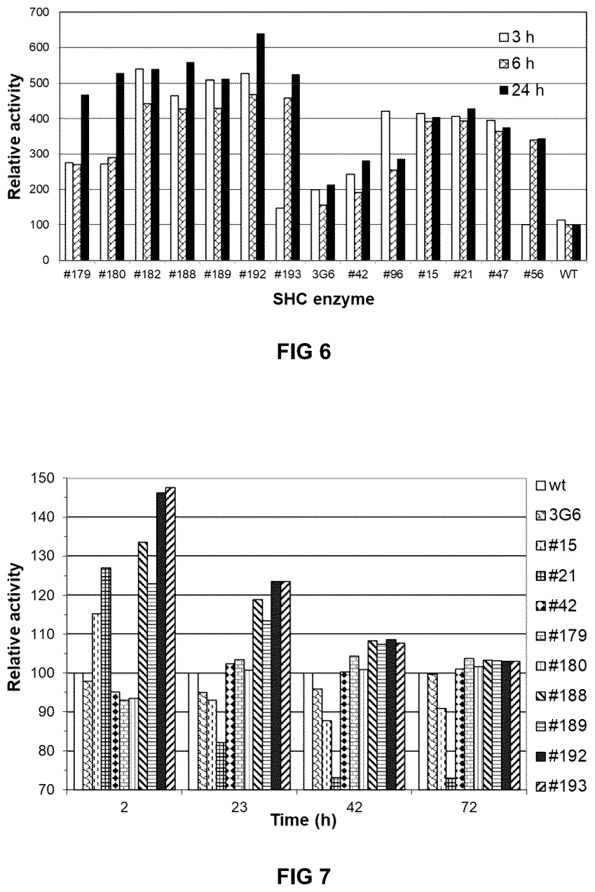

Cultivation and gene expression was done in microtiter plates as described for library screening (Example 1). SHC activity was assayed in 0.5 ml reactions containing 4 g/l E,Z-HFA; cells to an OD_{650 nm} of 10, 0.004% SDS in 50 mM succinic acid/NaOH buffer pH 5.2 (250 rpm). Reactions were incubated for 3, 6 or 24 hours at 35° C. and 250 rpm prior to solvent extraction and GC analysis. The mutations in selected additional variants are shown in Table 4, the activity of the variants (E,Z-HFA conversion relative to wt BmeSHC after 3, 6, and 24 h) is shown in FIG. 6.

SHC variants #179, #182, #188, #192, and #193 showed all between 4.5- and 6.5-fold improvement over wild-type BmeSHC (E,Z-HFA conversion after 24 hours of reaction).

TABLE 4
Mutations in selected BmeSHC variants

| | | | | | | | | SEQ ID | SEQ ID |
|---|---|---|---|---|---|---|---|---|---|
| SHC | | | | | | | | NO | NO |
| variant | Mutations | | | | | | | (DNA) | (AA) |
| 3G6 | I2N | T35A | A355T | L539H | | | | 3 | 4 |
| #179 | I2N | L5P | T35A | T166A | L539H | | | 29 | 30 |
| #180 | I2N | L5P | T35A | T166A | E211V | L539H | | 31 | 32 |
| #182 | I2N | L5P | T35A | E211V | S212R | Y483C | L539H | 33 | 34 |
| #188 | I2N | T166A | Y483C | | | | | 35 | 36 |
| #189 | I2N | T166A | Y483C | L539H | | | | 37 | 38 |
| #192 | I2N | T166A | E211V | Y483C | | | | 39 | 40 |
| #193 | I2N | T166A | E211V | Y483C | L539H | | | 41 | 42 |

### Example 4: Biocatalyst Production (Fermentation)

For SHC enzyme production in Escherichia coli the gene coding for the desired wild-type or variant squalene hopene cyclase enzyme was inserted into plasmid pET-28a (+), where it is under the control of an IPTG inducible T7-promoter. The plasmid was transformed into E. coli strain BL21 (DE3) using a standard heat-shock transformation procedure.

### Cultivation Medium

The minimal medium used as default for biocatalyst production contained
10% 10× citric acid/phosphate buffer (133 g/l KH₂PO₄, 40 g/l (NH₄)₂HPO₄, 17 g/l citric acid.H₂O in deionized water, with pH adjusted to 6.8 using 32% NaOH),
2.43% MgSO₄ solution (50% w/v MgSO₄·7H₂O in deionized water),
0.01% trace elements solution (50 g/l Na₂EDTA.2H₂O, 20 g/l FeSO₄·7H₂O, 3 g/l H₃BO₃, 0.9 g/l MnSO₄·2H₂O, 1.1 g/l CoCl₂, 80 g/l CuCl₂, 240 g/l NiSO₄·7H₂O, 100 g/l KI, 1.4 g/l (NH₄)₆MO₇O₂₄·4H₂O, 1 g/l ZnSO₄·7H₂O in deionized water),
0.01% Thiamin solution (2.25 g/l Thiamin. HCl in deionized water),
2% glucose solution (20% w/v glucose in deionized water).

The citric acid/phosphate buffer was first sterilized by autoclaving, the other ingredients added afterwards from sterile solutions sterilized either by autoclaving or filter-sterilization (0.2 μm).

### Fermentation

Fermentations were run in 750 ml InforsHT reactors. To the fermentation vessel was added 168 ml deionized water. The reaction vessel was equipped with all required probes (pO₂, pH, sampling, antifoam), C+N feed and sodium hydroxide bottles and autoclaved. After autoclaving is added to the reactor:
20 ml 10× phosphate/citric acid buffer
14 ml 50% glucose
0.53 ml MgSO₄ solution
2 ml (NH₄)₂SO₄ solution (50% (w/V) (NH₄)₂SO₄ in deionized water)
0.020 ml trace elements solution
0.400 ml thiamine solution
0.200 ml kanamycin solution (50 mg/ml)

The running parameters were as follows: pH=6.95, pO₂=40%, T=30° C., 300 rpm. Cascade: rpm setpoint at 300, min 300, max 1000, flow (l/min) set point 0.1, min 0, max 0.6. Antifoam control: 1:9.

A seed culture was grown in LB medium (+ Kanamycin) at 37° C., 220 rpm for 8 h. The fermenter was inoculated to an OD_{650 nm} of 0.4-0.5 from this seed culture. The fermentation was run first in batch mode for 11.5 h, where after was started the C+N feed with a feed solution (sterilized glucose solution (143 ml H₂O+35 g glucose) to which had been added after sterilization: 17.5 ml (NH₄)₂SO₄ solution, 1.8 ml MgSO₄ solution, 0.018 ml trace elements solution, 0.360 ml Thiamine solution, 0.180 ml kanamycin solution. The feed was run at a constant flow rate of approx. 4.2 ml/h. Glucose and NH₄⁺ measurements were done externally to evaluate availability of the C- and N-sources in the culture. Usually glucose levels stay very low.

Cultures were grown for a total of approx. 25 hours, where they reached typically an OD_{650 nm} of 40-45. SHC production was then induced by the addition of IPTG to a concentration of 1 mM to the fermenter, and lasted for approx. 16 h at 30° C. and pO₂=20%. At the end of induction, the cells were collected by centrifugation, washed with citric acid/sodium phosphate buffer pH 5.6 and stored as pellets at 4° C. or −20° C. until further use.

### Example 5: Optimized Reaction Conditions for BmeSHC Variants

The reaction conditions for selected SHC variants were individually optimized with regard to temperature, pH and SDS concentration. Biocatalysts were prepared by fermentation as described in Example 4.

Reactions of 2-5 ml volume with 4 g/l E,Z-HFA and cells (expressing variant SHC enzymes) loaded at an OD_{650 nm} of 10 were run in 0.1 M citric acid/sodium phosphate buffer pH 5.0-6.8, in presence of 0.010-0.020% SDS at temperatures ranging from 27 to 50° C. and under constant agitation (Heidolph synthesis 1 Liquid device, 800 rpm). Reaction conditions defined as optimized were confirmed/adjusted (pH) in 0.1 M succinic acid/NaOH buffer. The mutations introduced had some influence on SDS concentration optimum and pH over the variants. Main variations were observed relative to optimal temperature.

TABLE 5
Optimized reaction conditions for BmeSHC
wild type and variant enzymes¹.

| SHC enzyme | Temperature (° C.) | pH | [SDS] (w/v %)² |
|---|---|---|---|
| wt | 45° C. | 5.8 | 0.0025 |
| 3G6 | 40° C. | 5.8 | 0.015 |
| #15 | 35° C. | 5.8 | 0.015 |
| #21 | 35° C. | 5.8 | 0.015 |
| #42 | 35° C. | 5.8 | 0.015 |
| #47 | 35° C. | 5.8 | 0.015 |
| #56 | 35° C. | 5.8 | 0.015 |
| #96 | 35° C. | 5.8 | 0.015 |
| 59B7 | 35° C. | 5.6 | 0.015 |
| 13E9 | 40° C. | 5.8 | 0.020 |
| 50D3 | 40° C. | 5.8 | 0.020 |
| 73F9 | 35° C. | 5.8 | 0.015 |
| 83D1 | 35° C. | 5.8 | 0.020 |
| 114E1 | 40° C. | 5.8 | 0.020 |
| #179 | 30° C. | 5.6 | 0.014 |
| #180 | 30° C. | 6.0 | 0.012 |
| #182 | 30° C. | 5.6 | 0.014 |
| #188 | 30° C. | 5.8 | 0.012 |
| #189 | 30° C. | 5.8 | 0.012 |
| #192 | 30° C. | 5.8 | 0.012 |
| #193 | 30° C. | 5.8 | 0.012 |

¹The optimal values for wild type Bme SHC enzyme are provided for comparison purposes.
²In reactions containing cells to an OD_{650 nm} of 10.

### Example 6: Performance of SHC Variants in 135 g/l E,Z-Hydroxyfarnesylacetone Bioconversion

Biocatalysts produced by fermentation of the E. coli strains transformed with the plasmid carrying the gene coding for the selected BmeSHC wt or variant SHC enzymes were used in 135 g/l E,Z-HFA bioconversions. 4 ml reactions were run in Radleys Carousel Plus/Monoblock 16. They contained 135 g/E,Z-HFA, 182 g/l cells, and were run under conditions defined as optimal regarding temperature, pH, and SDS concentration.

FIG. 7 shows relative activity of wt and variant BmeSHC enzymes in terms of E,Z-HFA conversion to (+)-amberketal as a function of time. Full conversion was achieved with best variants #179, #189, #192, and #193 in 24-48 hours, whereas reaching full conversion with wt BmeSHC required 72 hours.

### Example 7: GC-FID Analysis

Samples were extracted (vigorous shaking) with an appropriate volume of MTBE for quantification of their content in substrate and reaction products. The solvent fraction was separated from the water phase by centrifugation prior to GC-FID analysis (table top centrifuge). 1 μl of the solvent phase was injected (split ratio 10) onto a 30 m×0.32 mm×0.25 μm DB-Wax column. The column was developed at constant flow (4 ml/min H₂) with the temperature gradient: 200° C., 25° C./min to 240° C., 120° C./min to 240° C., 4 min at 240° C. Split flow: 10 ml/min, split ratio: 5. Inlet temperature: 250° C., detector temperature: 150° C. This resulted in separation of E,Z-HFA and (+)-Amberketal. E,Z-HFA conversion was calculated from the areas of the (+)-Amberketal and E,Z-HFA peaks with the following formula:

$$EZHFA conversion ( \% ) = 100 \times ( {Area}_{Peak Amberketal} / ( {Area}_{Peak Amberketal} + {Area}_{EZHFA Peak} ) )$$

### Example 8: Cyclization of E,Z-Hydroxyfarnesylacetone

### E,Z-Hydroxyfarnesylacetone was Cyclized Using BmeSHC Variant #192.

The reaction contained 9.9 g E,Z-Hydroxyfarnesylacetone, 364 g/l cells that had produced BmeSHC variant #192, 1.15 g SDS (10% SDS) and was run in 0.1 M succinic acid/NaOH buffer pH 5.6 at 30° C. under constant agitation (115 ml total volume in a 250 ml flask, Radleys Monoblock). E,Z-hydroxyfarnesylacetone was fully converted in approx. 142 hours.

The reaction was extracted 5 times with 100 ml MTBE, the solvent phases recovered by centrifugation (30 min, 3579 g, room temperature), the solvent phases pooled, dried over MgSO₄, and the solvent evaporated by rotary evaporation, resulting into 20.9 g crude product.

The crude product was dissolved in ethanol, and crystallized by water addition. 8 g of crystalline (+)-amberketal of >99% purity according to GC analysis were recovered.

### Example 9: Cyclization of E,Z-Hydroxyfarnesylacetone from a Mixture of Hydroxyfarnesylacetone Isomers and Constitutional Isomers of Hydroxyfarnesylacetone

### A Mixture of the Following 4 Compounds was Cyclized Using BmeSHC Variant #192:

a) E,Z-isomer of compound of formula (II), wherein R was methyl (E,Z-hydroxyfarnesylacetone)
b) E,E-isomer of compound of formula (II), wherein R was methyl (E,E-hydroxyfarnesylacetone)
c) E,Z-isomer of compound of formula (IIa), wherein R was methyl
d) E,E-isomer of compound of formula (IIa), wherein R was methyl

The ratio of a:b:c:d in this Example was 37:9:29:16.

The reaction contained 135 g/l of the 4-compound-mixture and 364 g/l cells that had produced BmeSHC variant #192, 2.05 g SDS (10.25% SDS) and was run in 0.1 M succinic acid/NaOH buffer pH 5.6 at 30° C. under constant agitation (200 ml total volume in 250 ml DASBox fermenter). The reaction was run for a total of 150 hours, where E,Z-hydroxyfarnesylacetone conversion was approx. 80%.

The reaction was extracted 7 times with 100 ml MTBE, the solvent phases recovered by centrifugation (30 min, 3579 g, room temperature), pooled, dried over MgSO₄, and the solvent evaporated by rotary evaporation, resulting into 27.6 g crude product.

The reaction products were purified by flash chromatography using n-heptane/MTBE as the solvent system. The product-containing fractions were pooled and solvent evaporated, resulting into 7.1 g crude product.

The crude product was dissolved in ethanol and crystallized by water addition, resulting into 2 product fractions containing the compound of formula (I) and the compound of formula (V), wherein R was methyl.

The main product fraction (crystals, 5.4 g) contained the compound of formula (I) and the compound of formula (V) in a ratio 93:7 (>99% purity according to GC analysis).

A second product fraction (oily-crystalline, 708 mg) contained the compound of formula (I) and the compound of formula (V) in a ratio 42:58 (96.8% purity).

### Example 10: Mutations in Structural Elements Associated with Enzyme Stability

A model of the BmeSHC enzyme was created by means of homology modelling using the crystal structure of Alicyclobacillus acidocaldarius SHC (PDB ID: 2 SQC).

Structural elements influencing enzyme stability include but are not limited to e.g. glycine residues that might destabilize α-helices, or amino acid residues responsible for the formation of salt bridges.

Characteristic for the enzyme family of squalene hopene cyclases are QW-repeats (glutamine (Q)-tryptophane (W) motifs) that tighten the protein structure by an intricate interaction network (Wendt et al., The structure of the membrane protein squalene-hopene cyclase at 2.0 Å resolution, J. Mol. Biol 286, 175-187 (1999)).

Comparison of QW-repeats in BmeSHC and in homologs of BmeSHC resulted in the design of the BmeSHC #192 variants listed in Table 6 with mutations directed to the QW repeats.

TABLE 6
Mutations in structural elements responsible for enzyme stability.

| | | SeqID NO | SeqID NO |
|---|---|---|---|
| SHC variant | Mutations | (DNA) | (AA) |
| BmeSHC#192_v70 | F412W | 50 | 51 |
| BmeSHC#192_v71 | F530W | 52 | 53 |
| BmeSHC#192_v72 | F29W F412W | 54 | 55 |
| BmeSHC#192_v73 | F29W F412W F530W | 56 | 57 |
| BmeSHC#192_v75 | F412W F530W | 58 | 59 |

### Example 11: E,Z-Hydroxyfarnesylacetone Conversion with BmeSHC #192 Variants

Biocatalysts of the variants listed in Table 6 were produced by fermentation with the procedure described in Example 4.

For each of the variants, reaction conditions were individually optimized with the biocatalysts produced with respect to the reaction parameters temperature, pH and SDS concentration as described in Example 5. Optimized reaction conditions for selected BmeSHC #192 variants are listed in Table 7.

TABLE 7
Optimized reaction conditions for BmeSHC#192 variants.

| SHC enzyme | Temperature (° C.) | pH | [SDS] (w/v %)¹ |
|---|---|---|---|
| BmeSHC#192_v70 | 35 | 5.6-5.8 | 0.024 |
| BmeSHC#192_v71 | 35 | 5.6-6.2 | 0.018 |
| BmeSHC#192_v72 | 35 | 5.8-6.2 | 0.024 |
| BmeSHC#192_v73 | 35 | 5.6-6.2 | 0.018 |
| BmeSHC#192_v75 | 35 | 5.8-6.2 | 0.024 |

¹In reactions containing cells to an OD_{650 nm} of 10 (approx. 9 g/l cells).

Biocatalysts were used in 135 g/l E,Z-HFA bioconversions with 182 g/l cells: 4 ml reactions were run in Radleys Carousel Plus under conditions individually defined as optimal regarding temperature, pH, and SDS concentration for each of the variants.

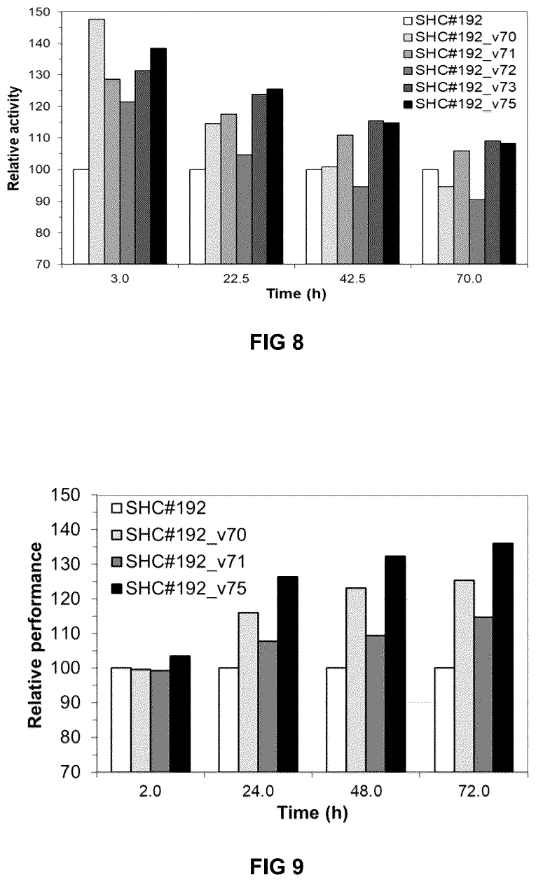

FIG. 8 shows the relative activity of parent and variant BmeSHC #192 enzymes in terms of E,Z-HFA conversion to (+)-amberketal as a function of time. Strengthening enzyme stability by means of addressing structural elements like QW-repeats allowed to increase enzymatic activity. The initial reaction velocity which was measured in terms of conversion after 3 hours of reaction was increased with all variants tested. E,Z-Hydroxyfarnesylacetone conversion after 42.5 and 70 h of reaction was higher with the variants compared to parent BmeSHC #192 other than the two variants BmeSHC #192_v70 and BmeSHC #192_v72.

### Example 12: EZ-Hydroxyfarnesylacetone Conversion with BmeSHC #192 Variants at a Cells:Substrate Ratio of 1

Biocatalysts of the variants BmeSHC #192_v70, BmeSHC #192_v71, and BmeSHC #192_v75 (Table 6) were produced by fermentation with the procedure described in Example 4. Biocatalysts were used in bioconversions with a cells:substrate ratio of 1 (100 g/l E,Z-HFA, 100 g/l cells): 4 ml reactions were run in Radleys Carousel Plus under conditions individually defined as optimal regarding temperature, pH, and SDS concentration for each of the variants (Table 7).

FIG. 9 shows the relative activity of parent and variant BmeSHC #192 enzymes measured in terms of E,Z-HFA conversion to (+)-amberketal as a function of time. Biocatalysts producing the variants BmeSHC #192_v70, BmeSHC #192_v71, and BmeSHC #192_v75 performed better than biocatalyst producing the parent enzyme BmeSHC #192: an increase in E,Z-HFA conversion of about 1.25-1.35-fold was observed with the variants over that of the parent enzyme.

1. A method for making a compound of formula (I) 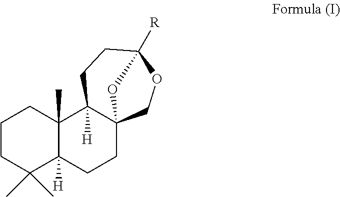
wherein the method comprises contacting a compound of formula (II) 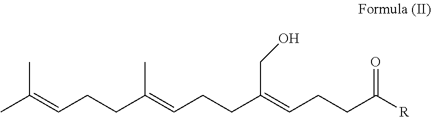
with a squalene-hopene cyclase (SHC) enzyme comprising an amino acid sequence having at least 70% identity or similarity with the sequence of SEQ ID NO: 1, wherein the SHC enzyme comprises one or more amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1,
and wherein R is selected from H and a C₁-C₄ alkyl.

2. The method according to claim 1, wherein the compound of formula (II) is such that the double bond between C-8 and C-9 is in E-configuration and the double bond between C-4 and C-5 is in Z-configuration (E,Z-isomer).

3. A method for making a mixture comprising a compound of formula (I) 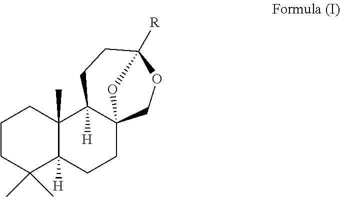
wherein the method comprises contacting a mixture comprising a compound of formula (II) and a compound of formula (IIa) 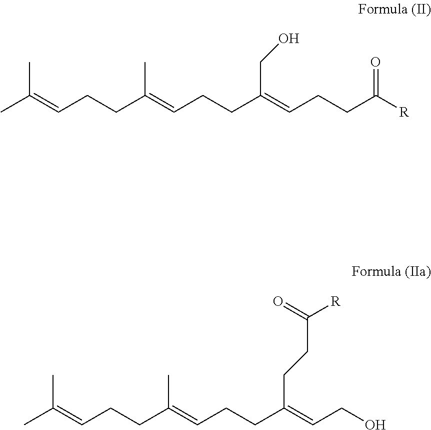
with a squalene-hopene cyclase (SHC) enzyme comprising an amino acid sequence having at least 70% identity or similarity with the sequence of SEQ ID NO: 1 or SEQ ID NOs: 43-49 and wherein R is selected from H and a C₁-C₄ alkyl.

4. The method according to claim 3, wherein the mixture comprising a compound of formula (I) further comprises a compound of formula (Ia) 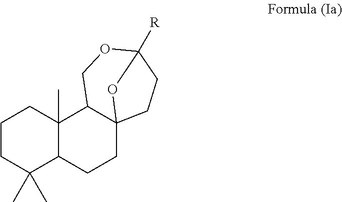
wherein R is selected from H and a C₁-C₄ alkyl.

5. The method according to claim 4, wherein the compound of formula (Ia) has the configuration of formula (V) 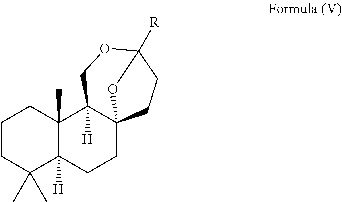
wherein R is selected from H and a C₁-C₄ alkyl.

6. The method according to claim 3,
wherein the mixture comprising a compound of formula (II) and a compound of formula (IIa) comprises any one of the following:
i) a compound of formula (II) that is such that the double bond between C-8 and C-9 is in E-configuration and the double bond between C-4 and C-5 is in Z-configuration (E,Z-isomer)
ii) a compound of formula (II) that is such that the double bond between C-8 and C-9 is in E-configuration and the double bond between C-4 and C-5 is in E-configuration (E,E-isomer)
iii) a compound of formula (IIa) that is such that the double bond between C-6 and C-7 is in E-configuration and the double bond between C-2 and C-3 is in Z-configuration (E,Z-isomer)
iv) a compound of formula (IIa) that is such that the double bond between C-6 and C-7 is in E-configuration and the double bond between C-2 and C-3 is in E-configuration (E,E-isomer)
v) a compound of formula (II) that is such that the double bond between C-8 and C-9 is in E-configuration and the double bond between C-4 and C-5 is in Z-configuration (E,Z-isomer) and a compound of formula (II) that is such that the double bond between C-8 and C-9 is in E-configuration and the double bond between C-4 and C-5 is in E-configuration (E,E-isomer)
vi) a compound of formula (IIa) that is such that the double bond between C-6 and C-7 is in E-configuration and the double bond between C-2 and C-3 is in Z-configuration (E,Z-isomer) and a compound of formula (IIa) that is such that the double bond between C-6 and C-7 is in E-configuration and the double bond between C-2 and C-3 is in E-configuration (E,E-isomer)
vii) any combination of i)-vi).

7. The method according to claim 3,
wherein the mixture comprising a compound of formula (II) and a compound of formula (IIa) comprises:
a compound of formula (II) that is such that the double bond between C-8 and C-9 is in E-configuration and the double bond between C-4 and C-5 is in Z-configuration (E,Z-isomer)
a compound of formula (II) that is such that the double bond between C-8 and C-9 is in E-configuration and the double bond between C-4 and C-5 is in E-configuration (E,E-isomer)
a compound of formula (IIa) that is such that the double bond between C-6 and C-7 is in E-configuration and the double bond between C-2 and C-3 is in Z-configuration (E,Z-isomer), and;
a compound of formula (IIa) that is such that the double bond between C-6 and C-7 is in E-configuration and the double bond between C-2 and C-3 is in E-configuration (E,E-isomer).

8. The method according to claim 1, wherein a compound of formula (III) 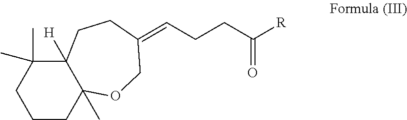
is made as a by-product,
wherein R is selected from H and a C₁-C₄ alkyl.

9. The method according to claim 1, wherein a compound having the relative configuration shown in formula (IIIa) is made as a by-product: 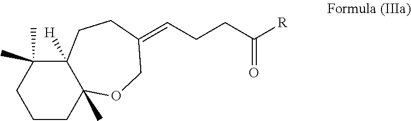
wherein R is selected from H and a C₁-C₄ alkyl.

10. The method according to claim 3, wherein a compound of formula (VI) 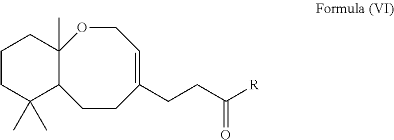
is made as a by-product,
wherein R is selected from H and a C₁-C₄ alkyl.

11. The method according to claim 3, wherein a compound having the relative configuration shown in formula (VIa) is made as a by-product: 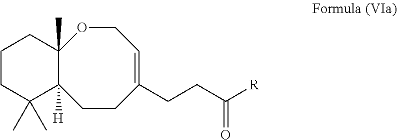
wherein R is selected from H and a C₁-C₄ alkyl.

12. The method according to claim 1, wherein R is methyl.

13. The method according to claim 1, wherein the SHC enzyme comprises an amino acid sequence having at least 70% identity or similarity with the sequence of SEQ ID NO: 1, and wherein the SHC enzyme comprises one to seven amino acid substitutions relative to SEQ ID NO: 1 at one or more positions corresponding to position 2, 5, 35, 116, 166, 211, 212, 317, 355, 382, 399, 483, 539, and 585 in SEQ ID NO: 1.

14. The method according to claim 1, wherein the SHC enzyme comprises one or more amino acid substitutions relative to SEQ ID NO: 1 at one or more positions corresponding to position 2, 5, 35, 166, 211, 212, 355, 483, and 539 in SEQ ID NO: 1.

15. The method according to claim 1, wherein the SHC enzyme comprises one or more amino acid substitutions relative to SEQ ID NO: 1 at one or more positions corresponding to position 2, 5, 35, 166, 211, 212, 483, and 539 in SEQ ID NO: 1.

16. The method according to claim 1, wherein the SHC enzyme comprises an amino acid substitution relative to SEQ ID NO: 1 selected from the following:
(i) an asparagine (N) residue at a position corresponding to position 2 in SEQ ID NO: 1;
(ii) a proline (P) residue at a position corresponding to position 5 in SEQ ID NO: 1;
(iii) an alanine (A) residue at a position corresponding to position 35 in SEQ ID NO: 1;
(iv) an threonine (T) residue at a position corresponding to position 116 in SEQ ID NO: 1;
(v) an alanine (A) residue at a position corresponding to position 166 in SEQ ID NO: 1;
(vi) a valine (V) residue at a position corresponding to position 211 in SEQ ID NO: 1;
(vii) an arginine (R) residue at a position corresponding to position 212 in SEQ ID NO: 1;
(viii) a methionine (M) residue at a position corresponding to position 317 in SEQ ID NO: 1;
(ix) a threonine (T) residue at a position corresponding to position 355 in SEQ ID NO: 1;
(x) a threonine (T) residue at a position corresponding to position 382 in SEQ ID NO: 1;
(xi) a valine (V) residue at a position corresponding to position 399 in SEQ ID NO: 1;
(xii) a cysteine (C) residue at a position corresponding to position 483 in SEQ ID NO: 1;
(xiii) a histidine (H) residue at a position corresponding to position 539 in SEQ ID NO: 1;
(xiv) an alanine (A) residue at a position corresponding to position 585 in SEQ ID NO: 1; or
(xv) any combination thereof.

17. The method according to claim 1, wherein the SHC enzyme comprises an amino acid substitution relative to SEQ ID NO: 1 selected from the following corresponding positions in SEQ ID NO: 1:
(i) I2N, T35A, A355T, and L539H;
(ii) T166A;
(iii) I2N and Y483C;
(iv) I2N, Y483C, and L539H;
(v) I2N, L5P, T35A, L539H;
(vi) I2N, L5P, T35A, and Y483C;
(vii) I2N, L5P, T35A, T166A, and L539H;
(viii) I2N, L5P, T35A, T166A, E211V, and L539H
(ix) I2N, L5P, T35A, E211V, S212R, Y483C, and L539H
(x) I2N, T166A, and Y483C;
(xi) I2N, T166A, Y483C, and L539H;
(xii) I2N, T166A, E211V, and Y483C; or
(xiii) I2N, T166A, E211V, Y483C, and L539H.

18. The method according to claim 1, wherein the SHC enzyme comprises the following amino acid substitutions relative to SEQ ID NO: 1: I2N and T166A.

19. The method according to claim 1, wherein the SHC enzyme further comprises one or more substitutions relative to SEQ ID NO: 1 selected from L5P, T35A, E211V, Y483C, and L539H.

20. The method according to claim 1, wherein the SHC enzyme further comprises an amino acid sequence that is at least 90% identical to any one of SEQ ID NOs: 4, 6, 8, 10, 12, 14, 16, 18, 20, 22, 24, 26, 28, 30, 32, 34, 36, 38, 40 or 42.

21. A nucleic acid molecule comprising a nucleotide sequence encoding a squalene hopene cyclase (SHC) enzyme as described in claim 1.

22. A vector comprising a nucleic acid molecule according to claim 21.

23. A host cell comprising a nucleic acid molecule according to claim 21.

24. A squalene hopene cyclase (SHC) enzyme as described in claim 1.

25. A composition comprising a compound of formula (I) and/or a compound of formula (Ia) 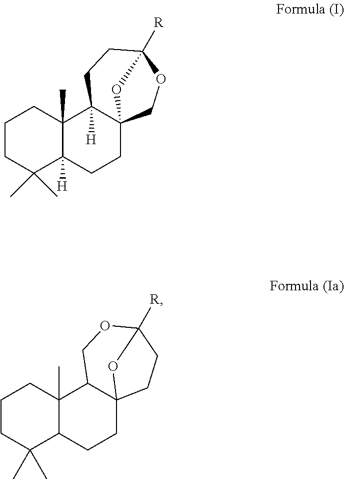
wherein said composition is obtained by the method of claim 4.

26. The composition according to claim 25, wherein the compound of formula (I) and/or the compound of formula (Ia) are in a solid form.

27. The composition according to claim 25, wherein the compound of formula (Ia) has the configuration of formula (V).

28. A method of manufacturing a fragrance composition or a consumer product, the method comprising adding the composition according to claim 25 to the fragrance composition or the consumer product.

29. A fragrance composition or a consumer product comprising the composition as defined in claim 25.

30. A mixture comprising the product obtainable obtained by the process of claim 3 wherein the mixture comprises I, Ia, III, IIIa, IV, IVa, V, Va VI, and/or VIa.

31. The composition according to claim 25 wherein the composition further comprises III, IIIa, IV, IVa, V, Va, VI and/or VIa.


SEQUENCE LISTING (59 sequences)

SEQ ID NO: 1 - protein, 625 aa, synthetic construct
  source 1..625: note = wt BmeSHC protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 2 - DNA, 1878 bp, synthetic construct
  source 1..1878: mol_type = other DNA; note = wt BmeSHC DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 3 - DNA, 1878 bp, synthetic construct
  source 1..1878: mol_type = other DNA; note = 3G6 DNA
atgaacattctgctcaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaattccacaacccaaataccacacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatctcacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 4 - protein, 625 aa, synthetic construct
  source 1..625: note = 3G6 protein
MNILLKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTTPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 5 - DNA, 1878 bp, synthetic construct
  source 1..1878: mol_type = other DNA; note = 59B7 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcttttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcgtcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 6 - protein, 625 aa, synthetic construct
  source 1..625: note = 59B7 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 7 - DNA, 1878 bp, synthetic construct
  source 1..1878: mol_type = other DNA; note = 13E9 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 8 - protein, 625 aa, synthetic construct
  source 1..625: note = 13E9 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 9 - DNA, 1878 bp, synthetic construct
  source 1..1878: mol_type = other DNA; note = 50D3 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcaccgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagacgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcaatgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagcgcaacaaacacattaccctaccggcattggcttgccgggccaattttacattcagtaccatagctacaatgatattttcccgttactggctctggcacattacgcgaagaagcatagcagctga

SEQ ID NO: 10 - protein, 625 aa, synthetic construct
  source 1..625: note = 50D3 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYITDSGGISRAHFLTRWMLSVNGLYEWPKLFYLPLSLLLVPTYVPLNFYELSTYARIHFVPMMVAGNKKFSLTSRHTPSLSHLDVREQKQESEETTQVRRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYAMQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTAQQTHYPTGIGLPGQFYIQYHSYNDIFPLLALAHYAKKHSS

SEQ ID NO: 11 - DNA, 1878 bp, synthetic construct
  source 1..1878: mol_type = other DNA; note = 73F9 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcgtcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 12 - protein, 625 aa, synthetic construct
  source 1..625: note = 73F9 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGVNWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 13 - DNA, 1878 bp, synthetic construct
  source 1..1878: mol_type = other DNA; note = 83D1 DNA
atgatcattctgcccaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgccgggaacaagaaattcagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggtatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 14 - protein, 625 aa, synthetic construct
  source 1..625: note = 83D1 protein
MIILPKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 15 - DNA, 1878 bp, synthetic construct
  source 1..1878: mol_type = other DNA; note = 114E1 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacttcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttaacacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 16 - protein, 625 aa, synthetic construct
  source 1..625: note = 114E1 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALTRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 17 - DNA, 1878 bp, synthetic construct
  source 1..1878: mol_type = other DNA; note = #15 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 18 - protein, 625 aa, synthetic construct
  source 1..625: note = #15 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 19 - DNA, 1878 bp, synthetic construct
  source 1..1878: mol_type = other DNA; note = #21 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 20 - protein, 625 aa, synthetic construct
  source 1..625: note = #21 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 21 - DNA, 1878 bp, synthetic construct
  source 1..1878: mol_type = other DNA; note = #42 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 22 - protein, 625 aa, synthetic construct
  source 1..625: note = #42 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 23 - DNA, 1878 bp, synthetic construct
  source 1..1878: mol_type = other DNA; note = #47 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 24 - protein, 625 aa, synthetic construct
  source 1..625: note = #47 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 25 - DNA, 1878 bp, synthetic construct
  source 1..1878: mol_type = other DNA; note = #56 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 26 - protein, 625 aa, synthetic construct
  source 1..625: note = #56 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 27 - DNA, 1878 bp, synthetic construct
  source 1..1878: mol_type = other DNA; note = #96 DNA
atgatcattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagacgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 28 - protein, 625 aa, synthetic construct
  source 1..625: note = #96 protein
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQVRRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 29 - DNA, 1878 bp, synthetic construct
  source 1..1878: mol_type = other DNA; note = #179 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 30 - protein, 625 aa, synthetic construct
  source 1..625: note = #179 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 31 - DNA, 1878 bp, synthetic construct
  source 1..1878: mol_type = other DNA; note = #180 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtacggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 32 - protein, 625 aa, synthetic construct
  source 1..625: note = #180 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 33 - DNA, 1878 bp, synthetic construct
  source 1..1878: mol_type = other DNA; note = #182 DNA
atgaacattctgcccaaggaagtccagctggagattcagcgccgcatcgcctatctgcgtccaacccagaagaatgacggttcgttccgctactgctttgaggcaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccacttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagacgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 34 - protein, 625 aa, synthetic construct
  source 1..625: note = #182 protein
MNILPKEVQLEIQRRIAYLRPTQKNDGSFRYCFEAGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQVRRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 35 - DNA, 1878 bp, synthetic construct
  source 1..1878: mol_type = other DNA; note = #188 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 36 - protein, 625 aa, synthetic construct
  source 1..625: note = #188 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 37 - DNA, 1878 bp, synthetic construct
  source 1..1878: mol_type = other DNA; note = #189 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagaaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 38 - protein, 625 aa, synthetic construct
  source 1..625: note = #190 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 39 - DNA, 1878 bp, synthetic construct
  source 1..1878: mol_type = other DNA; note = #192 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcactcaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 40 - protein, 625 aa, synthetic construct
  source 1..625: note = #192 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 41 - DNA, 1878 bp, synthetic construct
  source 1..1878: mol_type = other DNA; note = #193 DNA
atgaacattctgctcaaggaagtccagctggagattcagcgccgcatcgcctatctgcgtccaacccagaagaatgacggttcgttccgctactgctttgagacaggtgttatgcccgatgccttcctgatcatgcttctgcgcaccttcgatttagataaagaggttttaattaagcagcttacggaacgtattgtgagccttcaaaatgaagacggcctgtggaccctcttcgacgacgaggagcataacctcagcgcaacaattcaggcctacaccgccctcttatacagcggctattatcaaaagaatgaccgtatccttcgtaaagccgagcgctacatcatcgactctggcggtatttcccgcgcgcatttcctgacacgctggatgctgagtgtcaatggtttatatgaatggcctaaacttttctatctccctctgagcttgctgttggtgccaacctacgttccattaaatttctacgaactctccgcttatgctcgcatccatttcgtaccaatgatggttgctgggaacaagaaatttagtcttaccagccgccacaccccgagcttatcacaccttgatgtgcgcgagcagaaacaagaaagcgaagagacgacccaagtaagtcgtgcgagtatctttcttgttgaccacctcaagcaacttgcatcgttgcctagttatatccataagttgggctaccaagccgcagagcgttacatgcttgagcgtatcgagaaagatgggacactgtacagctacgcaacgtccaccttcttcatgatctacggccttctggccctgggctacaagaaagactcgttcgtaatccaaaaggcaattgatggcatctgttcacttctttcaacctgttcgggccacgtgcacgtcgaaaactcgacatcaacggtgtgggataccgcattgctgtcctatgcattgcaggaagccggtgtcccacaacaagatcctatgatcaaaggaactacccgctatctgaagaagcgccaacacactaagcttggggactggcaatttcacaacccaaataccgcacccggcggttggggtttctctgacattaatacaaataatccagacctggatgacacttctgcggccatccgtgcgttatcacgtcgcgcccaaacagacacggactacctggagtcctggcaacgtggcatcaattggcttctgtcgatgcaaaataaggacggtggcttcgcggcatttgagaagaacacggacagcattttgttcacgtaccttccactggaaaacgcgaaggacgccgcgaccgaccctgcgacggccgacctgaccgggcgtgtgttggagtgcttaggtaacttcgccggaatgaacaaatcacacccttctatcaaggccgccgtaaagtggctgttcgatcaccagttggataacggaagttggtgcggccgttggggcgtttgctacatctacgggacctgggccgcgatcacaggtttgcgcgcagttggggtgagcgcatcggacccacgtattatcaaggcgattaattggcttaagagtatccaacaggaagacggtggtttcggcgagtcttgttattcagcgtcacacaagaagtacgttcctttgtcattcagcaccccgagtcaaacggcctgggctctggacgccttaatgacgatctgtccgttaaaggaccaaagcgtggagaagggaatcaagttcttgctgaatccgaatttgacagagcaacaaacacattaccctaccggcattggcttgccgggccaattttacattcagtaccatagctacaatgatattttcccgttactggctctggcacattacgcgaagaagcatagcagctga

SEQ ID NO: 42 - protein, 625 aa, synthetic construct
  source 1..625: note = #193 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASHKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 43 - protein, 631 aa, synthetic construct [sequence omitted]

SEQ ID NO: 44 - protein, 608 aa, synthetic construct
  source 1..608: note = wt ZmoSHC1 protein
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQWARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYME

SEQ ID NO: 45 - protein, 658 aa, synthetic construct
  source 1..658: note = wt ZmoSHC2 protein
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYWARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGINWLAQNQDEEGLWKEDYYSGGGFPRVFYLRYHGYSKYFPLWALARYRNLKKANQPIVHYGM

SEQ ID NO: 46 - protein, 684 aa, synthetic construct
  source 1..684: note = wt BjaSHC protein
MTVTSSASARATRDPGNYQTALQSTVRAAADWLIANQKPDGHWVGRAESNACMEAQWCLALWFMGLEDHPLRKRLGQSLLDSQRPDGAWQVYFGAPNGDINATVEAYAALRSLGFRDDEPAVRRAREWIEAKGGLRNIRVFTRYWLALIGEWPWEKTPNIPPEVIWFPLWFPFSIYNFAQWARATLMPIAVLSARRPSRPLPPENRLDALFPHGRKAFDYELPVKAGAGGWDRFFRGADKVLHKLQNLGNRLNLGLFRPAATSRVLEWMIRHQDFDGAWGGIQPPWIYGLMALYAEGYPLNHPVLAKGLDALNDPGWRVDVGDATYIQATNSPVWDTILTLLAFDDAGVLGDYPEAVDKAVDWVLQRQVRVPGDWSMKLPHVKPGGWAFEYANNYYPDTDDTAVALIALAPLRHDPKWKAKGIDEAIQLGVDWLIGMQSQGGGWGAFDKDNNQKILTKIPFCDYGEALDPPSVDVTAHIIEAFGKLGISRNHPSMVQALDYIRREQEPSGPWFGRWGVNYVYGTGAVLPALAAIGEDMTQPYIGRACDWLVAHQQADGGWGESCASYMDVSAVGRGTTTASQTAWALMALLAANRPQDKDAIERGCMWLVERQSAGTWDEPEFTGTGFPGYGVGQTIKLNDPALSQRLMQGPELSRAFMLRYGMYRHYFPLMALGRALRPQSHS

SEQ ID NO: 47 - protein, 642 aa, synthetic construct
  source 1..642: note = wt TelSHC protein
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSWARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQPCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTGFPCHFYLKYHYYQQHFPLTALGRYARWRNLLAT

SEQ ID NO: 48 - protein, 720 aa, synthetic construct
  source 1..720: note = wt ApaSHC1 protein
MNMASRFSLKKILRSGSDTQGTNVNTLIQSGTSDIVRQKPAPQEPADLSALKAMGNSLTHTLSSACEWLMKQQKPDGHWVGSVGSNASMEAEWCLALWFLGLEDHPLRPRLGKALLEMQRPDGSWGTYYGAGSGDINATVESYAALRSLGYAEDDPAVSKAAAWIISKGGLKNVRVFTRYWLALIGEWPWEKTPNLPPEIIWFPDNFVFSIYNFAQWARATMMPLAILSARRPSRPLRPQDRLDALFPGGRANFDYELPTKEGRDVIADFFRLADKGLHWLQSSFLKRAPSREAAIKYVLEWIIWHQDADGGWGGIQPPWVYGLMALHGEGYQFHHPVMAKALDALNDPGWRHDKGDASWIQATNSPVWDTMLSLMALHDANAEERFTPEMDKALDWLLSRQVRVKGDWSVKLPNTEPGGWAFEYANDRYPDTDDTAVALIAIASCRNRPEWQAKGVEEAIGRGVRWLVAMQSSCGGWGAFDKDNNKSILAKIPFCDFGEALDPPSVDVTAHVLEAFGLLGLPRDLPCIQRGLAYIRKEQDPTGPWFGRWGVNYLYGTGAVLPALAALGEDMTQPYISKACDWLINCQQENGGWGESCASYMEVSSIGHGATTPSQTAWALMGLIAANRPQDYEAIAKGCRYLIDLQEEDGSWNEEEFTGTGFPGYGVGQTIKLDDPAISKRLMQGAELSRAFMLRYDLYRQLFPIIALSRASRLIKLGN

SEQ ID NO: 49 - protein, 685 aa, synthetic construct
  source 1..685: note = wt GmoSHC protein
MSPADISTKSSSFQRLDNMLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 50 - DNA, 1878 bp, synthetic construct
  source 1..1878: mol_type = other DNA; note = BmeSHC#192_v70 DNA
atgaacatcctgctgaaagaagtgcagctggaaattcagcgtcgtattgcatatctgcgcccgacccagaaaaatgatggcagttttcgttattgctttgaaaccggtgtgatgccggatgcctttctgattatgctgctgcgcacctttgatctggataaagaagttctgattaagcagctgaccgaacgcattgttagtctgcagaatgaagatggtctgtggaccctgtttgatgatgaagaacataatctgagcgcaaccattcaggcatataccgccctgctgtatagtggttattatcagaaaaatgaccgtattctgcgtaaagccgaacgttatattattgatagcggtggtattagccgtgcacattttctgacccgctggatgctgagcgtgaatggcctgtatgaatggccgaaactgttttatctgccgctgagcctgctgctggttccgacctatgttccgctgaatttttatgaactgagcgcctatgcacgtattcattttgtgccgatgatggtggcaggcaataagaaattttcactgaccagtcgtcataccccgagcctgagtcatctggatgttcgtgaacagaaacaggaaagcgaagaaaccacccaggttagtcgcgcaagcatttttctggtggatcatctgaaacagctggcaagtctgccgagttatattcataaactgggttatcaggcagccgaacgctatatgctggaacgcattgaaaaagatggcaccctgtatagttatgcaaccagtacctttttcatgatctatggcctgctggccctgggctataaaaaagatagctttgttattcagaaggcaattgatggtatttgtagtctgctgagtacctgcagtggccatgttcatgtggaaaatagcaccagtaccgtgtgggataccgccctgttaagctatgccctgcaggaagcaggtgttccgcagcaggatccgatgattaagggtaccacccgttatctgaaaaaacgtcagcataccaaactgggtgactggcagtttcataatccgaataccgccccgggtggctggggttttagtgatattaataccaataacccggatctggatgataccagtgcagccattcgcgccctgagtcgccgcgctcagaccgataccgattatctggaaagttggcagcgtggtattaattggctgctgagtatgcagaataaggatggtggttgggcagcctttgaaaagaataccgatagcattctgtttacctatctgccgttagaaaatgcaaaagatgccgcaaccgatccggccaccgccgatctgaccggccgtgttctggaatgcctgggtaattttgcaggcatgaataagagtcatccgagcattaaggccgcagtgaaatggctgtttgatcatcagctggataatggtagttggtgtggccgttggggtgtgtgttatatctatggtacctgggcagccattaccggtctgcgtgcagtgggcgtgagtgcaagcgatccgcgtattattaaggcaattaattggttaaagagcatccagcaggaagatggcggttttggcgaaagctgttatagtgcaagtctgaaaaaatatgtgccgctgagttttagtaccccgagccagaccgcctgggcactggatgcactgatgaccatttgtccgctgaaagatcagagtgtggaaaaaggcattaagtttctgctgaatccgaatctgaccgaacagcagacccattatccgaccggtattggtctgccgggtcagttttatattcagtatcatagttacaacgacatctttccgctgctggcactggcccattatgccaaaaaacatagcagctaa

SEQ ID NO: 51 - protein, 625 aa, synthetic construct
  source 1..625: note = BmeSHC#192_v70 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGWAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 52 - DNA, 1878 bp, synthetic construct
  source 1..1878: mol_type = other DNA; note = BmeSHC#192_v71 DNA
atgaacatcctgctgaaagaagtgcagctggaaattcagcgtcgtattgcatatctgcgcccgacccagaaaaatgatggcagttttcgttattgctttgaaaccggtgtgatgccggatgcctttctgattatgctgctgcgcacctttgatctggataaagaagttctgattaagcagctgaccgaacgcattgttagtctgcagaatgaagatggtctgtggaccctgtttgatgatgaagaacataatctgagcgcaaccattcaggcatataccgccctgctgtatagtggttattatcagaaaaatgaccgtattctgcgtaaagccgaacgttatattattgatagcggtggtattagccgtgcacattttctgacccgctggatgctgagcgtgaatggcctgtatgaatggccgaaactgttttatctgccgctgagcctgctgctggttccgacctatgttccgctgaatttttatgaactgagcgcctatgcacgtattcattttgtgccgatgatggtggcaggcaataagaaattttcactgaccagtcgtcataccccgagcctgagtcatctggatgttcgtgaacagaaacaggaaagcgaagaaaccacccaggttagtcgcgcaagcatttttctggtggatcatctgaaacagctggcaagtctgccgagttatattcataaactgggttatcaggcagccgaacgctatatgctggaacgcattgaaaaagatggcaccctgtatagttatgcaaccagtacctttttcatgatctatggcctgctggccctgggctataaaaaagatagctttgttattcagaaggcaattgatggtatttgtagtctgctgagtacctgcagtggccatgttcatgtggaaaatagcaccagtaccgtgtgggataccgccctgttaagctatgccctgcaggaagcaggtgttccgcagcaggatccgatgattaagggtaccacccgttatctgaaaaaacgtcagcataccaaactgggtgactggcagtttcataatccgaataccgccccgggtggctggggttttagtgatattaataccaataacccggatctggatgataccagtgcagccattcgcgccctgagtcgccgcgctcagaccgataccgattatctggaaagttggcagcgtggtattaattggctgctgagtatgcagaataaggatggtggttttgcagcctttgaaaagaataccgatagcattctgtttacctatctgccgttagaaaatgcaaaagatgccgcaaccgatccggccaccgccgatctgaccggccgtgttctggaatgcctgggtaattttgcaggcatgaataagagtcatccgagcattaaggccgcagtgaaatggctgtttgatcatcagctggataatggtagttggtgtggccgttggggtgtgtgttatatctatggtacctgggcagccattaccggtctgcgtgcagtgggcgtgagtgcaagcgatccgcgtattattaaggcaattaattggttaaagagcatccagcaggaagatggcggttggggcgaaagctgttatagtgcaagtctgaaaaaatatgtgccgctgagttttagtaccccgagccagaccgcctgggcactggatgcactgatgaccatttgtccgctgaaagatcagagtgtggaaaaaggcattaagtttctgctgaatccgaatctgaccgaacagcagacccattatccgaccggtattggtctgccgggtcagttttatattcagtatcatagttacaacgacatctttccgctgctggcactggcccattatgccaaaaaacatagcagctaa

SEQ ID NO: 53 - protein, 625 aa, synthetic construct
  source 1..625: note = BmeSHC#192_v71 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGWGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 54 - DNA, 1878 bp, synthetic construct
  source 1..1878: mol_type = other DNA; note = BmeSHC#192_v72 DNA
atgaacatcctgctgaaagaagtgcagctggaaattcagcgtcgtattgcatatctgcgcccgacccagaaaaatgatggcagttggcgttattgctttgaaaccggtgtgatgccggatgcctttctgattatgctgctgcgcacctttgatctggataaagaagttctgattaagcagctgaccgaacgcattgttagtctgcagaatgaagatggtctgtggaccctgtttgatgatgaagaacataatctgagcgcaaccattcaggcatataccgccctgctgtatagtggttattatcagaaaaatgaccgtattctgcgtaaagccgaacgttatattattgatagcggtggtattagccgtgcacattttctgacccgctggatgctgagcgtgaatggcctgtatgaatggccgaaactgttttatctgccgctgagcctgctgctggttccgacctatgttccgctgaatttttatgaactgagcgcctatgcacgtattcattttgtgccgatgatggtggcaggcaataagaaattttcactgaccagtcgtcataccccgagcctgagtcatctggatgttcgtgaacagaaacaggaaagcgaagaaaccacccaggttagtcgcgcaagcatttttctggtggatcatctgaaacagctggcaagtctgccgagttatattcataaactgggttatcaggcagccgaacgctatatgctggaacgcattgaaaaagatggcaccctgtatagttatgcaaccagtacctttttcatgatctatggcctgctggccctgggctataaaaaagatagctttgttattcagaaggcaattgatggtatttgtagtctgctgagtacctgcagtggccatgttcatgtggaaaatagcaccagtaccgtgtgggataccgccctgttaagctatgccctgcaggaagcaggtgttccgcagcaggatccgatgattaagggtaccacccgttatctgaaaaaacgtcagcataccaaactgggtgactggcagtttcataatccgaataccgccccgggtggctggggttttagtgatattaataccaataacccggatctggatgataccagtgcagccattcgcgccctgagtcgccgcgctcagaccgataccgattatctggaaagttggcagcgtggtattaattggctgctgagtatgcagaataaggatggtggttgggcagcctttgaaaagaataccgatagcattctgtttacctatctgccgttagaaaatgcaaaagatgccgcaaccgatccggccaccgccgatctgaccggccgtgttctggaatgcctgggtaattttgcaggcatgaataagagtcatccgagcattaaggccgcagtgaaatggctgtttgatcatcagctggataatggtagttggtgtggccgttggggtgtgtgttatatctatggtacctgggcagccattaccggtctgcgtgcagtgggcgtgagtgcaagcgatccgcgtattattaaggcaattaattggttaaagagcatccagcaggaagatggcggttttggcgaaagctgttatagtgcaagtctgaaaaaatatgtgccgctgagttttagtaccccgagccagaccgcctgggcactggatgcactgatgaccatttgtccgctgaaagatcagagtgtggaaaaaggcattaagtttctgctgaatccgaatctgaccgaacagcagacccattatccgaccggtattggtctgccgggtcagttttatattcagtatcatagttacaacgacatctttccgctgctggcactggcccattatgccaaaaaacatagcagctaa

SEQ ID NO: 55 - protein, 625 aa, synthetic construct
  source 1..625: note = BmeSHC#192_v72 protein
MNILLKEVQLEIQRRIAYLRPTQKNDGSWRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGWAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 56 - DNA, 1878 bp, synthetic construct
  source 1..1878: mol_type = other DNA; note = BmeSHC#192_v73 DNA
atgaacatcctgctgaaagaagtgcagctggaaattcagcgtcgtattgcatatctgcgcccgacccagaaaaatgatggcagttggcgttattgctttgaaaccggtgtgatgccggatgcctttctgattatgctgctgcgcacctttgatctggataaagaagttctgattaagcagctgaccgaacgcattgttagtctgcagaatgaagatggtctgtggaccctgtttgatgatgaagaacataatctgagcgcaaccattcaggcatataccgccctgctgtatagtggttattatcagaaaaatgaccgtattctgcgtaaagccgaacgttatattattgatagcggtggtattagccgtgcacattttctgacccgctggatgctgagcgtgaatggcctgtatgaatggccgaaactgttttatctgccgctgagcctgctgctggttccgacctatgttccgctgaatttttatgaactgagcgcctatgcacgtattcattttgtgccgatgatggtggcaggcaataagaaattttcactgaccagtcgtcataccccgagcctgagtcatctggatgttcgtgaacagaaacaggaaagcgaagaaaccacccaggttagtcgcgcaagcatttttctggtggatcatctgaaacagctggcaagtctgccgagttatattcataaactgggttatcaggcagccgaacgctatatgctggaacgcattgaaaaagatggcaccctgtatagttatgcaaccagtacctttttcatgatctatggcctgctggccctgggctataaaaaagatagctttgttattcagaaggcaattgatggtatttgtagtctgctgagtacctgcagtggccatgttcatgtggaaaatagcaccagtaccgtgtgggataccgccctgttaagctatgccctgcaggaagcaggtgttccgcagcaggatccgatgattaagggtaccacccgttatctgaaaaaacgtcagcataccaaactgggtgactggcagtttcataatccgaataccgccccgggtggctggggttttagtgatattaataccaataacccggatctggatgataccagtgcagccattcgcgccctgagtcgccgcgctcagaccgataccgattatctggaaagttggcagcgtggtattaattggctgctgagtatgcagaataaggatggtggttgggcagcctttgaaaagaataccgatagcattctgtttacctatctgccgttagaaaatgcaaaagatgccgcaaccgatccggccaccgccgatctgaccggccgtgttctggaatgcctgggtaattttgcaggcatgaataagagtcatccgagcattaaggccgcagtgaaatggctgtttgatcatcagctggataatggtagttggtgtggccgttggggtgtgtgttatatctatggtacctgggcagccattaccggtctgcgtgcagtgggcgtgagtgcaagcgatccgcgtattattaaggcaattaattggttaaagagcatccagcaggaagatggcggttggggcgaaagctgttatagtgcaagtctgaaaaaatatgtgccgctgagttttagtaccccgagccagaccgcctgggcactggatgcactgatgaccatttgtccgctgaaagatcagagtgtggaaaaaggcattaagtttctgctgaatccgaatctgaccgaacagcagacccattatccgaccggtattggtctgccgggtcagttttatattcagtatcatagttacaacgacatctttccgctgctggcactggcccattatgccaaaaaacatagcagctaa

SEQ ID NO: 57 - protein, 625 aa, synthetic construct
  source 1..625: note = BmeSHC#192_v73 protein
MNILLKEVQLEIQRRIAYLRPTQKNDGSWRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGWAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGWGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 58 - DNA, 1878 bp, synthetic construct
  source 1..1878: mol_type = other DNA; note = BmeSHC#192_v75 DNA
atgaacatcctgctgaaagaagtgcagctggaaattcagcgtcgtattgcatatctgcgcccgacccagaaaaatgatggcagttttcgttattgctttgaaaccggtgtgatgccggatgcctttctgattatgctgctgcgcacctttgatctggataaagaagttctgattaagcagctgaccgaacgcattgttagtctgcagaatgaagatggtctgtggaccctgtttgatgatgaagaacataatctgagcgcaaccattcaggcatataccgccctgctgtatagtggttattatcagaaaaatgaccgtattctgcgtaaagccgaacgttatattattgatagcggtggtattagccgtgcacattttctgacccgctggatgctgagcgtgaatggcctgtatgaatggccgaaactgttttatctgccgctgagcctgctgctggttccgacctatgttccgctgaatttttatgaactgagcgcctatgcacgtattcattttgtgccgatgatggtggcaggcaataagaaattttcactgaccagtcgtcataccccgagcctgagtcatctggatgttcgtgaacagaaacaggaaagcgaagaaaccacccaggttagtcgcgcaagcatttttctggtggatcatctgaaacagctggcaagtctgccgagttatattcataaactgggttatcaggcagccgaacgctatatgctggaacgcattgaaaaagatggcaccctgtatagttatgcaaccagtacctttttcatgatctatggcctgctggccctgggctataaaaaagatagctttgttattcagaaggcaattgatggtatttgtagtctgctgagtacctgcagtggccatgttcatgtggaaaatagcaccagtaccgtgtgggataccgccctgttaagctatgccctgcaggaagcaggtgttccgcagcaggatccgatgattaagggtaccacccgttatctgaaaaaacgtcagcataccaaactgggtgactggcagtttcataatccgaataccgccccgggtggctggggttttagtgatattaataccaataacccggatctggatgataccagtgcagccattcgcgccctgagtcgccgcgctcagaccgataccgattatctggaaagttggcagcgtggtattaattggctgctgagtatgcagaataaggatggtggttgggcagcctttgaaaagaataccgatagcattctgtttacctatctgccgttagaaaatgcaaaagatgccgcaaccgatccggccaccgccgatctgaccggccgtgttctggaatgcctgggtaattttgcaggcatgaataagagtcatccgagcattaaggccgcagtgaaatggctgtttgatcatcagctggataatggtagttggtgtggccgttggggtgtgtgttatatctatggtacctgggcagccattaccggtctgcgtgcagtgggcgtgagtgcaagcgatccgcgtattattaaggcaattaattggttaaagagcatccagcaggaagatggcggttggggcgaaagctgttatagtgcaagtctgaaaaaatatgtgccgctgagttttagtaccccgagccagaccgcctgggcactggatgcactgatgaccatttgtccgctgaaagatcagagtgtggaaaaaggcattaagtttctgctgaatccgaatctgaccgaacagcagacccattatccgaccggtattggtctgccgggtcagttttatattcagtatcatagttacaacgacatctttccgctgctggcactggcccattatgccaaaaaacatagcagctaa

SEQ ID NO: 59 - protein, 625 aa, synthetic construct
  source 1..625: note = BmeSHC#192_v75 protein
MNILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSAYARIHFVPMMVAGNKKFSLTSRHTPSLSHLDVREQKQESEETTQVSRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGWAAFEKNTDSILFTYLPLENAKDAATDPATADLTGRVLECLGNFAGMNKSHPSIKAAVKWLFDHQLDNGSWCGRWGVCYIYGTWAAITGLRAVGVSASDPRIIKAINWLKSIQQEDGGWGESCYSASLKKYVPLSFSTPSQTAWALDALMTICPLKDQSVEKGIKFLLNPNLTEQQTHYPTGIGLPGQFYIQYHSYNDIFPLLALAHYAKKHSS